SEQUENCE LISTING (4 sequences)

SEQ ID NO: 1 - protein, 281 aa, Homo sapiens [sequence omitted]

SEQ ID NO: 2 - protein, 168 aa, synthetic construct
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 3 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAREKIQDLLVDSGKFIYLEDGTGSAVSLE

SEQ ID NO: 4 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

(19) United States
(12) Patent Application Publication
(10) Pub. No.: US 2026/0151504 A1
(43) Pub. Date: Jun. 4, 2026

(54) SYNTHETIC CELLS COMPRISING TNF-RELATED APOPTOSIS-INDUCING LIGAND (TRAIL) AND METHODS OF USING THE SAME

(71) Applicant: Slingshot Biosciences, Inc., Emeryville, CA (US)

(72) Inventors: Harini KETHAR, Danville, CA (US); Sunil THOMAS, San Bruno, CA (US); Juan ARMAS, Emeryville, CA (US); Daixuan ZHANG, Emeryville, CA (US); Subhanip BISWAS, Emeryville, CA (US); Kanwal PALLA, Oakland, CA (US)

(21) Appl. No.: 19/413,235

(22) Filed: Dec. 9, 2025

Related U.S. Application Data
(63) Continuation of application No. PCT/US24/35136, filed on Jun. 21, 2024.
(60) Provisional application No. 63/509,785, filed on Jun. 23, 2023.

(51) Int. Cl. A61K 47/69; A61K 47/62

(52) U.S. Cl. CPC A61K 47/6927; A61K 47/62; A61K 47/6903

(57) ABSTRACT

The disclosure provides synthetic cytotoxic cells, comprising a hydrogel microsphere and a TNF-related apoptosis-inducing ligand (TRAIL), or active fragment thereof. In aspects, the TRAIL or active fragment thereof is attached to the surface of the hydrogel microsphere. The disclosure further provides methods of preparing the synthetic cytotoxic cells and methods of use thereof in the treatment of cancer.

### CROSS REFERENCE TO RELATED APPLICATIONS

The present Application is a continuation of International Patent Application PCT/US2024/035136, filed on Jun. 21, 2024, which claims the benefit of priority to U.S. Provisional Application No. 63/509,785, filed on Jun. 23, 2023, each of which is hereby incorporated by reference in its entirety.

### REFERENCE TO AN ELECTRONIC SEQUENCE LISTING

The contents of the electronic sequence listing (47357-720.301_ST26.xml; Size: 5,900 bytes; and Date of Creation: Jun. 21, 2024) are herein incorporated by reference in its entirety.

### FIELD

The present disclosure generally relates to synthetic cells, comprising TNF-related apoptosis-inducing ligand (TRAIL). The present disclosure also relates to methods of using the same for treating a disorder, such as a cancer.

### BACKGROUND

TNF-related apoptosis-inducing ligand (TRAIL) is a type II transmembrane ligand protein of the tumor necrosis factor (TNF) cytokine super family, which can induce apoptosis in cancer cells while largely sparing non-cancerous cells. Notably, TRAIL is expressed by immune cells, including NK cells and T cells, while TRAIL receptors, such as death receptor 4 (DR4) and DR5, are highly expressed by many kinds of cancer cells. Promoting the interaction between TRAIL and the TRAIL receptors DR4 and DR5 can promote downstream induction of cancer cell apoptosis.

Given TRAIL's ability to specifically kill cancer cells, studies have tried to harness this ability to develop cancer therapeutics. For instance, soluble TRAIL protein and antibody-directed TRAIL receptors have been tested for their therapeutic potential. However, these approaches were met with limited, if any, success due to the poor agonistic ability of these agents.

Thus, there is an unmet need to develop effective TRAIL-mediated therapeutic approaches that show enhanced cytotoxicity towards cancer cells.

### SUMMARY

The present disclosure provides synthetic cytotoxic cells, comprising a hydrogel microsphere with a surface and a TNF-related apoptosis-inducing ligand (TRAIL), or active fragment thereof. In aspects, the TRAIL or active fragment thereof is attached to the surface of the hydrogel microsphere. In aspects, the hydrogel microsphere is a porous hydrogel microsphere. In aspects, the hydrogel microsphere is a smooth hydrogel microsphere. In aspects, the hydrogel microsphere comprises polymerized acrylamide and bis-acrylamide.

In aspects, the TRAIL or active fragment thereof comprises an amino acid sequence having at least 80%, at least 85%, at least 90%, at least 95%, at least 96%, at least 97%, at least 98%, at least 99%, or 100% identity to SEQ ID NO: 1. In aspects, the TRAIL or active fragment thereof is linked to an Fc domain. In aspects, the TRAIL or active fragment thereof is biotinylated. In aspects, the surface of the microsphere comprises a streptavidin molecule. In aspects, the TRAIL or active fragment thereof is biotinylated and the surface of the hydrogel microsphere comprises a streptavidin molecule. In aspects, the biotinylated TRAIL or active fragment thereof is attached to the surface of the hydrogel microsphere via interaction with the streptavidin molecule. In aspects, the synthetic cytotoxic cell comprises a multimer of the TRAIL or active fragment thereof. In aspects, the synthetic cytotoxic cell comprises a trimer of the TRAIL or active fragment thereof. In aspects, the diameter of the synthetic cytotoxic cell ranges from about 1 μm to about 45 μm. In aspects, the diameter of the synthetic cytotoxic cell is about 10 μm. In aspects, the synthetic cytotoxic cell exhibits a mean Young's modulus in the range of about 0.2 kPa and about 400 kPa.

The disclosure further provides a population of the synthetic cytotoxic cells disclosed herein. In aspects, the polydispersity index of the population is less than 0.05. The disclosure also provides compositions comprising the synthetic cytotoxic cells disclosed herein. In aspects, the composition comprises a pharmaceutically acceptable excipient, carrier, or diluent.

The disclosure provides methods of inducing apoptosis and/or necrosis of a target cell, comprising contacting the target cell with the synthetic cytotoxic cells disclosed herein, the populations of synthetic cytotoxic cells disclosed herein, or the compositions disclosed herein, thereby inducing apoptosis and/or necrosis of the target cell. In aspects, the contacting of the target cell is performed in vitro, ex vivo, or in vivo. In aspects, the contacting of the target cell is performed in vivo. In aspects, the method comprises administering the synthetic cytotoxic cell or the composition to a subject in need thereof

The disclosure provides methods of inducing apoptosis and/or necrosis of a target cell in a subject in need thereof, comprising administering the synthetic cytotoxic cells disclosed herein, the populations of synthetic cytotoxic cells disclosed herein, or the compositions disclosed herein to the subject, thereby inducing apoptosis and/or necrosis of the target cell in the subject. In aspects, the administering results in contacting of the target cell with the synthetic cytotoxic cell in vivo.

The disclosure provides methods of treating a cancer in a subject in need thereof, comprising administering the synthetic cytotoxic cells disclosed herein, the populations of synthetic cytotoxic cells disclosed herein, or the compositions disclosed herein to the subject. In aspects, the method comprises inducing apoptosis and/or necrosis of a target cell in the subject. In aspects, the target cell expresses one or both of (a) a TRAIL receptor DR4; and (b) a TRAIL receptor DR5. In aspects, the cell expresses a TRAIL receptor DR4.

In aspects, the TRAIL receptor DR4 comprises an amino acid sequence having at least 80%, at least 85%, at least 90%, at least 95%, at least 96%, at least 97%, at least 98%, at least 99%, or 100% identity to SEQ ID NO: 3. In aspects, the cell expresses a TRAIL receptor DR5. In aspects, the TRAIL receptor DR5 comprises an amino acid sequence having at least 80%, at least 85%, at least 90%, at least 95%, at least 96%, at least 97%, at least 98%, at least 99%, or 100% identity to SEQ ID NO: 4. In aspects, the method results in an interaction between: (i) TRAIL or active fragment thereof and DR4; (ii) TRAIL or active fragment thereof and DR5; or (iii) both (i) and (ii).

In aspects, the cancer is a leukemia, a lymphoma, a myeloma, a carcinoma, a sarcoma, a brain cancer, a spinal cord cancer, chronic lymphocytic leukemia (CLL), B cell acute lymphocytic leukemia (B-ALL), acute lymphoblastic leukemia (ALL), acute myeloid leukemia (AML), non-Hodgkin's lymphoma (NHL), diffuse large cell lymphoma (DLCL), diffuse large B cell lymphoma (DLBCL), Hodgkin's lymphoma, multiple myeloma, renal cell carcinoma (RCC), hepatocellular carcinoma, melanoma, mesothelioma, colorectal cancer, bladder cancer, breast cancer, colorectal cancer, ovarian cancer, prostate cancer, lung cancer, esophageal cancer, pancreatic cancer, head and neck cancer, liver cancer, cervical cancer, breast cancer, astrocytoma, medulloblastoma, neuroblastoma, non-small cell lung cancer, peritoneal carcinomatosis, a solid tumor, malignant pleura mesothelioma, gastric cancer, urothelial cancer, cholangiocarcinoma, hepatocellular carcinoma, or any combination thereof. In aspects, the subject is human.

In aspects, the method results in a proportion of apoptotic and/or necrotic target cells that is higher than the proportion of apoptotic and/or necrotic target cells resulting from contacting the target cell with a soluble form of TRAIL or active fragment thereof. In aspects, the method results in a proportion of apoptotic and/or necrotic target cells that is higher than the proportion of apoptotic and/or necrotic target cells resulting from contacting the target cell with a comparator microsphere, wherein the comparator microsphere comprises a non-hydrogel microsphere and the TRAIL or active fragment thereof, wherein the TRAIL or active fragment thereof is attached to the surface of the non-hydrogel microsphere.

In aspects, the method results in a proportion of apoptotic and/or necrotic target cells that is higher than the proportion of apoptotic and/or necrotic target cells resulting from contacting the target cell with a comparator microsphere, wherein the comparator microsphere comprises a microsphere and TRAIL or active fragment thereof, wherein the TRAIL or active fragment thereof is encapsulated by the microsphere. In aspects, the comparator microsphere comprises a microsphere comprised of poly (lactic-co-glycolic acid). In aspects, the target cell is a cancer cell.

The disclosure further provides conjugates comprising a cell and any of the synthetic cytotoxic cells disclosed herein. The disclosure also provides cells, wherein the cell is conjugated to any one of the synthetic cytotoxic cells disclosed herein. The disclosure further provides mixtures of (i) a cell, and a (ii) any of the synthetic cytotoxic cells disclosed herein.

In aspects, the cell and the synthetic cytotoxic cell are non-covalently conjugated. In aspects, the cell expresses one or both of: (a) a TRAIL receptor DR4 and (b) a TRAIL receptor DR5. In aspects, the cell expresses a TRAIL receptor DR4. In aspects, the TRAIL receptor DR4 comprises an amino acid sequence having at least 80%, at least 85%, at least 90%, at least 95%, at least 96%, at least 97%, at least 98%, at least 99%, or 100% identity to SEQ ID NO: 3. In aspects, the cell expresses a TRAIL receptor DR5, or an antigen binding fragment thereof. In aspects, the TRAIL receptor DR5 comprises an amino acid sequence having at least 80%, at least 85%, at least 90%, at least 95%, at least 96%, at least 97%, at least 98%, at least 99%, or 100% identity to SEQ ID NO: 4.

In aspects, the cell is conjugated to the synthetic cytotoxic cell via an interaction between: (i) the TRAIL or active fragment thereof and DR4; (ii) the TRAIL or active fragment thereof and DR5; or (iii) both (i) and (ii). In aspects, the interaction between the TRAIL or active fragment thereof, and one or both of DR4 and DR5 results in the induction of apoptosis and/or necrosis of the cell. In aspects, the cell is a cancer cell.

The disclosure also provides synthetic cytotoxic cell, comprising a porous hydrogel microsphere and a TNF-related apoptosis-inducing ligand (TRAIL), or active fragment thereof, wherein the TRAIL or active fragment thereof is attached to the surface of the hydrogel microsphere. The disclosure provides a method of inducing apoptosis and/or necrosis of a target cell, comprising contacting the target cell with the synthetic cytotoxic cells disclosed herein that comprise a porous hydrogel microsphere, thereby inducing apoptosis and/or necrosis of the target cell. In aspects, contacting of the target cell is performed in vivo. In aspects, the method comprises administering the synthetic cytotoxic cell, the composition, or the pharmaceutical composition to a subject in need thereof.

In aspects, the method results in a proportion of apoptotic and/or necrotic target cells that is higher than the proportion of apoptotic and/or necrotic target cells resulting from contacting the target cell with a comparator microsphere, wherein the comparator microsphere comprises a smooth porous microsphere and a TNF-related apoptosis-inducing ligand (TRAIL), or active fragment thereof, wherein the TRAIL or active fragment thereof is attached to the surface of the smooth porous microsphere.

In aspects, the method results in a proportion of early apoptotic target cells that is higher than the proportion of early or late apoptotic target cells resulting from contacting the target cell with a soluble form of TRAIL or active fragment thereof.

In aspects, the method results in a proportion of early apoptotic target cells that is higher than the proportion of early or late apoptotic target cells resulting from contacting the target cell with a comparator microsphere, wherein the comparator microsphere comprises a non-hydrogel microsphere and a TNF-related apoptosis-inducing ligand (TRAIL), or active fragment thereof, wherein the TRAIL or active fragment thereof is attached to the surface of the non-hydrogel microsphere.

In aspects, the method results in a proportion of early apoptotic target cells that is higher than the proportion of early or late apoptotic target cells resulting from contacting the target cell with a comparator microsphere, wherein the comparator microsphere comprises a microsphere and a TNF-related apoptosis-inducing ligand (TRAIL), or active fragment thereof, wherein the TRAIL or active fragment thereof is encapsulated by the microsphere, optionally, wherein the microsphere is comprised of poly (lactic-co-glycolic acid).

In aspects, the method results in a proportion of early apoptotic target cells that is higher than the proportion of early or late apoptotic target cells resulting from contacting the target cell with a comparator microsphere, wherein the comparator microsphere comprises a smooth porous microsphere and a TNF-related apoptosis-inducing ligand (TRAIL), or active fragment thereof, wherein the TRAIL or active fragment thereof is attached to the surface of the smooth porous microsphere.

In aspects, the method results in a proportion of late apoptotic target cells that is higher than the proportion of early or late apoptotic target cells resulting from contacting the target cell with a soluble form of TRAIL or active fragment thereof.

In aspects, the method results in a proportion of late apoptotic target cells that is higher than the proportion of early or late apoptotic target cells resulting from contacting the target cell with a comparator microsphere, wherein the comparator microsphere comprises a non-hydrogel microsphere and a TNF-related apoptosis-inducing ligand (TRAIL), or active fragment thereof, wherein the TRAIL or active fragment thereof is attached to the surface of the non-hydrogel microsphere.

In aspects, the method results in a proportion of late apoptotic target cells that is higher than the proportion of early or late apoptotic target cells resulting from contacting the target cell with a comparator microsphere, wherein the comparator microsphere comprises a microsphere and a TNF-related apoptosis-inducing ligand (TRAIL), or active fragment thereof, wherein the TRAIL or active fragment thereof is encapsulated by the microsphere, optionally, wherein the microsphere is comprised of poly (lactic-co-glycolic acid).

In aspects, the method results in a proportion of late apoptotic target cells that is higher than the proportion of early or late apoptotic target cells resulting from contacting the target cell with a comparator microsphere, wherein the comparator microsphere comprises a smooth porous microsphere and a TNF-related apoptosis-inducing ligand (TRAIL), or active fragment thereof, wherein the TRAIL or active fragment thereof is attached to the surface of the smooth porous microsphere.

The disclosure also provides synthetic cytotoxic cells, comprising a) a hydrogel microsphere with a surface; and b) a TRAIL receptor selected from the group consisting of DR4 and DR5; wherein the TRAIL receptor is tethered to the surface of the hydrogel microsphere. The disclosure also provides biological complexes, comprising: a) a hydrogel microsphere with a surface; and b) a biological cell comprising a TRAIL receptor selected from the group consisting of DR4 and DR5; wherein the biological cell is tethered to the hydrogel microsphere via the TRAIL receptor.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows a confocal image of the purified porous hydrogel microspheres (prepared with 9 wt. % PEG).

FIG. 2 shows a confocal image of the purified 85:15 PLGA microspheres.

FIG. 3 schematically illustrates TRAIL-mediated killing of TRAIL-receptor-expressing cancer cells by synthetic cytotoxic cells having surface-attached TRAIL.

FIG. 4A shows FACS plots representing apoptotic and necrotic Jeko-1 MCL within 24 hours of co-culture with porous hydrogel synthetic cells not conjugated with TRAIL. FIG. 4B shows FACS plots representing apoptotic and necrotic Jeko-1 MCL within 24 hours of co-culture with TRAIL-conjugated porous hydrogel synthetic cells.

FIG. 5A shows FACS plots representing apoptotic and necrotic Jeko-1 MCL within 24 hrs of co-culture with 85:15 PLGA synthetic cells not conjugated with TRAIL. FIG. 5B shows FACS plots representing apoptotic and necrotic Jeko-1 MCL within 24 hrs of co-culture with TRAIL-conjugated 85:15 PLGA synthetic cells.

FIGS. 6A-6C show brightfield images of 5.5 μm STV magnetic beads (FIG. 6A), 20 μm streptavidin (STV) porous hydrogel microspheres (FIG. 6B), and 20 μm STV “smooth” hydrogel microspheres (FIG. 6C).

FIGS. 7A-C are quantitative fluorescent intensity flow plots reflecting the quantitation of TRAIL encapsulated in PLGA microspheres. FIG. 7A is a flow plot showing the capture by STV porous hydrogel microspheres of biotinylated TRAIL released from disrupted PLGA microspheres. The biotinylated TRAIL was labeled with anti-TRAIL antibody (APC). FIG. 7B shows a flow plot from a negative control experiment where STV porous hydrogel microspheres were used together with blank PLGA microspheres which do not contain any encapsulated TRAIL. FIG. 7C shows a flow plot of a negative control experiment comprising running blank porous beads on the same flow cytometer acquisition settings. This negative control is used to determine scatter match and distinguishing porous beads shown in FIG. 7A.

FIG. 8 shows an example gating scheme used to determine the percentage of apoptotic cells upon treatment of TRAIL-expressing tumor cells, Jeko-1 and Jurkat, with synthetic cytotoxic cells disclosed herein.

FIGS. 9A-9H are flow plots depicting the percentage of Annexin V-positive Jeko-1 tumor cells upon treatment for 24 hours with either control conditions (0.01% DMSO; FIG. 9A, and blank beads; FIG. 9B), or with PLGA microspheres encapsulating TRAIL (FIG. 9C), TRAIL attached to the surface of porous hydrogel microspheres (FIG. 9D), TRAIL attached to the surface of smooth hydrogel microspheres (FIG. 9E), TRAIL attached to the surface of magnetic beads (FIG. 9F), soluble TRAIL (FIG. 9G), or 100 nM camptothecin (FIG. 9H).

FIGS. 10A-10I are flow plots depicting the percentage of 7AAD-positive vs. Annexin V-positive Jeko-1 tumor cells upon treatment for 24 hours with either control conditions (0.01% DMSO; FIG. 10A, and blank beads; FIG. 10B), or with PLGA microspheres encapsulating TRAIL (FIG. 10C), TRAIL attached to the surface of porous hydrogel microspheres (FIG. 10D), TRAIL attached to the surface of smooth hydrogel microspheres (FIG. 10E), TRAIL attached to the surface of magnetic beads (FIG. 10F), soluble TRAIL (FIG. 10G), or 100 nM camptothecin (FIG. 10H). FIG. 101 shows the expected distribution of necrotic cells, late-stage apoptotic cells, live cells, and early-stage apoptotic cells in the four quadrants of the graphs shown in FIGS. 10A-H, FIGS. 12A-12H.

FIGS. 11A-11H are flow plots depicting the percentage of Annexin V-positive Jurkat tumor cells upon treatment for 24 hours with either control conditions (0.01% DMSO; FIG. 11A, and blank beads; FIG. 11B), or with PLGA microspheres encapsulating TRAIL (FIG. 11C), TRAIL attached to the surface of porous hydrogel microspheres (FIG. 11D), TRAIL attached to the surface of smooth hydrogel microspheres (FIG. 11E), TRAIL attached to the surface of magnetic beads (FIG. 11F), soluble TRAIL (FIG. 11G), or 100 nM camptothecin (FIG. 11H).

FIGS. 12A-12H are flow plots depicting percentage of 7AAD-positive vs. Annexin V-positive Jurkat tumor cells upon treatment for 24 hours with either control conditions (0.01% DMSO; FIG. 12A, and blank beads; FIG. 12B), or with PLGA microspheres encapsulating TRAIL (FIG. 12C), TRAIL attached to the surface of porous hydrogel microspheres (FIG. 12D), TRAIL attached to the surface of smooth hydrogel microspheres (FIG. 12E), TRAIL attached to the surface of magnetic beads (FIG. 12F), soluble TRAIL (FIG. 12G), or 100 nM camptothecin (FIG. 12H).

FIGS. 13A-13H are flow plots depicting the percentage of CD19-positive vs. Annexin V-positive Jeko-1 tumor cells upon treatment for 48 hours with either control conditions (0.01% DMSO; FIG. 13A, and blank beads; FIG. 13B), or with PLGA microspheres encapsulating TRAIL (FIG. 13C), TRAIL attached to the surface of porous hydrogel microspheres (FIG. 13D), TRAIL attached to the surface of smooth hydrogel microspheres (FIG. 13E), TRAIL decorated on magnetic beads (FIG. 13F), soluble TRAIL (FIG. 13G), or 100 nM camptothecin (FIG. 13H).

FIGS. 14A-14H are flow plots depicting the percentage of Annexin V-positive vs. 7AAD-positive Jeko-1 tumor cells upon treatment for 48 hours with either control conditions (0.01% DMSO; FIG. 14A, and blank beads; FIG. 14B), or with PLGA microspheres encapsulating TRAIL (FIG. 14C), TRAIL attached to the surface of porous hydrogel microspheres (FIG. 14D), TRAIL attached to the surface of smooth hydrogel microspheres (FIG. 14E), TRAIL attached to the surface of magnetic beads (FIG. 14F), soluble TRAIL (FIG. 14G), or 100 nM camptothecin (FIG. 14H).

FIGS. 15A-15H are flow plots depicting percentage of Annexin V-positive vs. CD3-positive Jurkat tumor cells upon treatment for 48 hours with either control conditions (0.01% DMSO; FIG. 15A, and blank beads; FIG. 15B), or with PLGA microspheres encapsulating TRAIL (FIG. 15C), TRAIL attached to the surface of porous hydrogel microspheres (FIG. 15D), TRAIL attached to the surface of smooth hydrogel microspheres (FIG. 15E), TRAIL attached to the surface of magnetic beads (FIG. 15F), soluble TRAIL (FIG. 15G), or 100 nM camptothecin (FIG. 15H).

FIGS. 16A-16H are flow plots depicting the percentage of Annexin V-positive vs. 7-AAD-positive Jurkat tumor cells upon treatment for 48 hours with either control conditions (0.01% DMSO; FIG. 16A, and blank beads; FIG. 16B), or with PLGA microspheres encapsulating TRAIL (FIG. 16C), TRAIL attached to the surface of porous hydrogel microspheres (FIG. 16D), TRAIL attached to the surface of smooth hydrogel microspheres (FIG. 16E), TRAIL attached to the surface of magnetic beads (FIG. 16F), soluble TRAIL (FIG. 16G), or 100 nM camptothecin (FIG. 16H).

### DETAILED DESCRIPTION

### Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa unless the content clearly dictates otherwise. In the event that any description of a term set forth conflicts with any document incorporated herein by reference, the description of the term set forth below shall control.

Following long-standing patent law convention, the terms “a”, “an”, and “the” refer to “one or more” when used in this application, including the claims, unless the context clearly dictates otherwise.

Unless otherwise indicated, it is to be understood that all numbers expressing quantities, ratios, and numerical properties of ingredients, reaction conditions, and so forth, used in the specification are contemplated to be able to be modified in all instances by the term “about”.

Unless otherwise indicated, it is to be understood that all numbers expressing quantities, ratios, and numerical properties of ingredients, reaction conditions, and so forth, used in the specification are contemplated to be able to be modified in all instances by the term “including all ranges and subranges therebetween”.

Throughout this application, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents. In aspects, “about” is used to indicate that a value includes the inherent variation of error for the device or the method being employed to determine the value, or the variation that exists among the samples being measured.

The term “including all ranges and subranges therebetween” or equivalents, are used herein to denote the intention that disclosure of any range or series of possible values, inherently also discloses all ranges and subranges encompassed by the highest and lowest values disclosed. This term includes the entire range from highest to lowest disclosed values, as well as subranges from any two or more disclosed points. This term is also intended to disclose any subranges encompassed anywhere within the highest and lowest disclosed values, including between two points that are explicitly recited in the document, up to one decimal point. Thus, disclosure of values 0, 5, 10, 15, 20, including all ranges and subranges therebetween, should be interpreted as also encompassing a range from 0-20, a range from 0-5 or 5-15, as well as a range from 2-16, or 3.1 to 19.8, etc.

The term “between”, as used in a phrase as such “between A and B” or “between A-B” refers to a range including both A and B.

Also as used herein, “and/or” refers to and encompasses any and all possible combinations of one or more of the associated listed items, as well as the lack of combinations when interpreted in the alternative (“or”).

The terms “including”, “includes”, “included”, and other forms, as used herein, are not limiting.

As used herein, the term “contacting” a cell with a substance (i.e., contacting a cell with a synthetic cytotoxic cell) includes, but is not limited to, an interaction between the cell and the substance (e.g., a synthetic cytotoxic cell). Such an interaction may be direct (e.g., involving contact between the surface of a cell and the surface of a synthetic cytotoxic cell), or may be indirect (e.g., mediated by a cell surface-attached protein and a protein attached to the surface of a synthetic cytotoxic cell). In aspects, contacting a cell with a substance comprises incubating the substance and the cell together in vitro (e.g., adding the synthetic cytotoxic cell to cells in culture). In aspects, “contacting” comprises administering a substance to a subject resulting in the contact between the substance and a cell of the subject in vivo.

As used herein, microspheres are small spherical particles ranging from 1 μm to 1000 μm. In general, microspheres may be prepared using natural or synthetic polymers using methods, such as single emulsion, double emulsion, phase separation, spray drying, and ionotropic gelation. A microsphere comprising a hydrogel is referred to herein as a “hydrogel microsphere”. A microsphere that does not comprise a hydrogel is referred to herein as a “non-hydrogel microsphere”. Exemplary non-hydrogel microspheres include magnetic beads as discussed herein.

The term “hydrogel”, as used herein, refers to a three-dimensional macromolecular material that has the ability to swell in the presence of water and to shrink in the absence of (or due to reduction in the amount of) water, but not dissolve in water. In aspects, the swelling, i.e., the absorption of water, is a consequence of the presence of hydrophilic functional groups attached to or dispersed within the macromolecular network. Crosslinks between adjacent macromolecules result in the aqueous insolubility of these hydrogels. The cross-links may be due to chemical (i.e., covalent) or physical (i.e., VanDer Waal forces, hydrogen-bonding, ionic forces, etc.) bonds. Synthetic hydrogels can be prepared by polymerizing a monomeric material to form a backbone and cross-linking the backbone with a crosslinking agent. A hydrogel may be in a dehydrated state or in a hydrated state. A characteristic of a hydrogel is that the material retains the general shape, whether dehydrated or hydrated. Thus, if the hydrogel has an approximately spherical shape in the dehydrated condition, it will be spherical in the hydrated condition.

As may be used herein, the term “macropore” refers to porous structures within the microspheres that are larger than those naturally formed during the polymerization of the one or more monomer materials. Typically, macropores are created by first incorporating one or more porogens during the preparation of microspheres and then removing the porogen(s) from the microspheres. The diameters of macropores usually exceed 50 nm. In aspects, the mean diameter of the macropores is between about 200 nm and about 2 μm.

As may be used herein, the term “micropore” refers to porous structures within the microspheres that are naturally formed during the polymerization of the one or more monomer materials. The sizes of the micropores are typically small, with a diameter in the low nanometer range. The diameters of micropores rarely exceed 50 nm. In aspects, the mean diameter of the micropores is between about 1 nm and about 20 nm. In aspects, the mean diameter of the micropores is between about 2 nm and about 4 nm.

As used herein, a “porous hydrogel microsphere” is a hydrogel microsphere comprising macropores and micropores. An exemplary method of generation of a porous hydrogel microsphere is described in Example 4 and includes the use of a porogen to generate macropores.

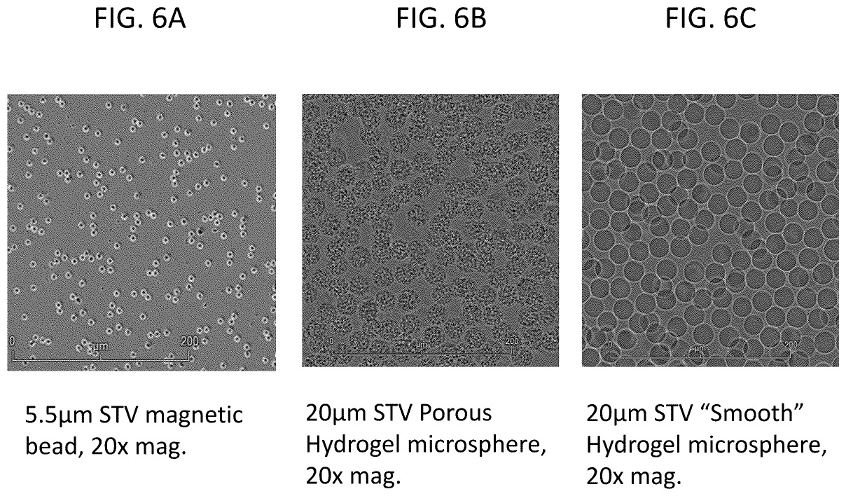

As used herein, a “smooth hydrogel microsphere” is a hydrogel microsphere comprising micropores, but lacking macropores. An exemplary method of generation of a smooth hydrogel microsphere is described in Example 4 and excludes the use of a porogen. In aspects, the surface of the smooth hydrogel microspheres appears smoother than that of porous hydrogel microsphere using brightfield microscopy (compare e.g., FIGS. 6B, 6C).

The term “multimer”, as used herein, refers to an aggregate of two or more single unit molecules (i.e., monomer) that is held together with non-covalent or covalent bonds. In some embodiments, the multimer is a dimer. In some embodiments, the multimer is a trimer.

The term “synthetic”, as applied to a nucleic acid, a polypeptide, a cell, or an organism, refers to a nucleic acid, polypeptide, cell, or organism that cannot be directly isolated from a source in nature.

The term “substantially similar,” as used herein, denotes at least 40% similar, at least 50% similar, at least 60% similar, at least 70% similar, at least 80% similar, at least 90% similar, at least 95% similar, at least 96% similar, at least 97% similar, at least 98% similar, or at least 99% similar.

The term “polydispersity index” (PDI), as used herein, refers to a measure of the heterogeneity of size, surface area, and/or mass of molecules or particles (such as microspheres) in a population. As used herein, PDI is calculated as Mw/Mn where Mw is the weight average molecular weight of the molecule or particle, and Mn is number average molecular weight of the molecule or particle. In general, a population of molecules or particles with a higher value of PDI has a broader molecular weight distribution. As discussed herein, the PDI of a population of synthetic cytotoxic cells disclosed herein is very low (e.g., in the range of 0 to about 0.1), indicating that the synthetic cytotoxic cells disclosed herein have a more homogenous distribution of sizes in the population.

The term “percent identity” in the context of two or more nucleic acid or polypeptide sequences, refers to two or more sequences or subsequences that have a specified percentage of nucleotides or amino acid residues that are the same, when compared. Unless otherwise indicated, percent identity is determined using the National Center for Biotechnology Information (NCBI)'s Basic Local Alignment Search Tool (BLAST®) using default parameters, available at blast.ncbi.nlm.nih.gov/Blast.cgi, version BLAST+2.13.0.

The term “pharmaceutical composition” or “therapeutic composition”, as used herein, refers to a composition capable of being administered to a subject. In aspects, the administration is intended for the treatment of a particular disease or disorder.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, or allergic response.

The term “pharmaceutically acceptable excipient, carrier or diluent”, as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington, The Science and Practice of Pharmacy, 23rd edition, A. Adejare, ed., Academic Press, 2020).

The term “subject”, as used herein, refers to an “animal” and in particular a “mammal” such as a non-primate (e.g., mice, rats, bovines, horses, household cats, tigers and other large cats, dogs, pigs, rabbits, goats, deer, sheep, ferrets, gerbils, guinea pigs, hamsters, bats, and birds (e.g., chickens, turkeys, and ducks)) or a primate (e.g., monkeys, baboons, chimpanzees, and human). The term may be used interchangeably with the term “patient” or “individual”. In some embodiments, the subject is a mammal, e.g., a human, diagnosed with a disease or disorder disclosed herein (e.g., a cancer). In some embodiments, the subject is a mammal, e.g., a human, at risk of developing a disease or disorder disclosed herein (e.g., a cancer). In some embodiments, the subject is human.

The terms “treatment” and “treating”, as used herein, refer to a pharmaceutical or other intervention regimen for obtaining beneficial or desired results in the recipient. Beneficial or desired results include but are not limited to a therapeutic benefit and/or a prophylactic benefit. Therapeutic benefit refers to any therapeutically relevant improvement in or effect on one or more diseases, conditions, or symptoms under treatment. The term “treating” in aspects, includes: (1) preventing or delaying the appearance of clinical symptoms of the state, disorder, or condition developing in the patient that may be afflicted with or predisposed to the state, disorder, or condition but does not yet experience or display clinical or subclinical symptoms of the state, disorder, or condition; (2) inhibiting the state, disorder, or condition (e.g., arresting, reducing, or delaying the development of the disease, or a relapse thereof in case of maintenance treatment, of clinical or subclinical symptom thereof); (3) relieving the condition (for example, by causing regression, or reducing the severity of the state, disorder, or condition or of its clinical or subclinical symptoms). In aspects, therapeutic benefit may refer to eradication or amelioration of symptoms of an underlying disorder being treated. In aspects, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like.

### Synthetic Cytotoxic Cells

The disclosure provides synthetic cytotoxic cells comprising, a hydrogel microsphere with a surface and a TNF-related apoptosis-inducing ligand (TRAIL), or active fragment thereof. In aspects, the TRAIL or the active fragment thereof is attached to the surface of the hydrogel microsphere. Without being bound by a theory, it is thought that the synthetic cytotoxic cells disclosed herein promote interaction between the TRAIL protein (or the active fragment thereof) and TRAIL death receptors (such as DR4 and DR5) on cells (such as cancer cells), thereby leading to enhanced cytotoxicity effects. Due to this enhanced cytotoxicity of the synthetic cytotoxic cells disclosed herein, lower dosage levels of therapeutic compositions comprising such synthetic cytotoxic cells may be sufficient to promote beneficial therapeutic effects.

In aspects, the average diameter of the synthetic cytotoxic cell is about 1 μm to about 50 μm. In aspects, the average diameter of the synthetic cytotoxic cell is at least about 1 μm. In aspects, the average diameter of the synthetic cytotoxic cell is at most about 50 μm. In aspects, the average diameter of the synthetic cytotoxic cell is about 1 μm to about 5 μm, about 1 μm to about 10 μm, about 1 μm to about 15 μm, about 1 μm to about 20 μm, about 1 μm to about 25 μm, about 1 μm to about 30 μm, about 1 μm to about 35 μm, about 1 μm to about 40 μm, about 1 μm to about 45 μm, about 1 μm to about 50 μm, about 5 μm to about 10 μm, about 5 μm to about 15 μm, about 5 μm to about 20 μm, about 5 μm to about 25 μm, about 5 μm to about 30 μm, about 5 μm to about 35 μm, about 5 μm to about 40 μm, about 5 μm to about 45 μm, about 5 μm to about 50 μm, about 10 μm to about 15 μm, about 10 μm to about 20 μm, about 10 μm to about 25 μm, about 10 μm to about 30 μm, about 10 μm to about 35 μm, about 10 μm to about 40 μm, about 10 μm to about 45 μm, about 10 μm to about 50 μm, about 15 μm to about 20 μm, about 15 μm to about 25 μm, about 15 μm to about 30 μm, about 15 μm to about 35 μm, about 15 μm to about 40 μm, about 15 μm to about 45 μm, about 15 μm to about 50 μm, about 20 μm to about 25 μm, about 20 μm to about 30 μm, about 20 μm to about 35 μm, about 20 μm to about 40 μm, about 20 μm to about 45 μm, about 20 μm to about 50 μm, about 25 μm to about 30 μm, about 25 μm to about 35 μm, about 25 μm to about 40 μm, about 25 μm to about 45 μm, about 25 μm to about 50 μm, about 30 μm to about 35 μm, about 30 μm to about 40 μm, about 30 μm to about 45 μm, about 30 μm to about 50 μm, about 35 μm to about 40 μm, about 35 μm to about 45 μm, about 35 μm to about 50 μm, about 40 μm to about 45 μm, about 40 μm to about 50 μm, or about 45 μm to about 50 μm. In aspects, the average diameter of the synthetic cytotoxic cell is about 1 μm, about 5 μm, about 10 μm, about 15 μm, about 20 μm, about 25 μm, about 30 μm, about 35 μm, about 40 μm, about 45 μm, or about 50 μm. In aspects, the average diameter of the synthetic cytotoxic cell is about 1 μm to about 45 μm. In aspects, the average diameter of the synthetic cytotoxic cell is about 10 μm.

In aspects, the synthetic cytotoxic cells disclosed herein exhibit a Young's modulus in the range of about 0.2 kPa to about 400 kPa, for instance about 1 kPa, about 5 kPa, about 10 kPa, about 20 kPa, about 50 kPa, about 100 kPa, about 150 kPa, about 200 kPa, about 250 kPa, about 300 kPa, about 350 kPa, or about 400 kPa, including all values and subranges that lie therebetween. In aspects, the synthetic cytotoxic cells further comprise fluorophore.

The disclosure further provides a population of the synthetic cytotoxic cells disclosed herein. In aspects, the polydispersity index of the population is less than 0.05. In aspects, the polydispersity index of the population is about 0 to about 0.1. In aspects, the polydispersity index of the population is about 0. In aspects, the polydispersity index of the population is at most about 0.1. In aspects, the polydispersity index of the population is about 0 to about 0.002, about 0 to about 0.004, about 0 to about 0.006, about 0 to about 0.008, about 0 to about 0.01, about 0 to about 0.02, about 0 to about 0.04, about 0 to about 0.06, about 0 to about 0.08, about 0 to about 0.1, about 0.002 to about 0.004, about 0.002 to about 0.006, about 0.002 to about 0.008, about 0.002 to about 0.01, about 0.002 to about 0.02, about 0.002 to about 0.04, about 0.002 to about 0.06, about 0.002 to about 0.08, about 0.002 to about 0.1, about 0.004 to about 0.006, about 0.004 to about 0.008, about 0.004 to about 0.01, about 0.004 to about 0.02, about 0.004 to about 0.04, about 0.004 to about 0.06, about 0.004 to about 0.08, about 0.004 to about 0.1, about 0.006 to about 0.008, about 0.006 to about 0.01, about 0.006 to about 0.02, about 0.006 to about 0.04, about 0.006 to about 0.06, about 0.006 to about 0.08, about 0.006 to about 0.1, about 0.008 to about 0.01, about 0.008 to about 0.02, about 0.008 to about 0.04, about 0.008 to about 0.06, about 0.008 to about 0.08, about 0.008 to about 0.1, about 0.01 to about 0.02, about 0.01 to about 0.04, about 0.01 to about 0.06, about 0.01 to about 0.08, about 0.01 to about 0.1, about 0.02 to about 0.04, about 0.02 to about 0.06, about 0.02 to about 0.08, about 0.02 to about 0.1, about 0.04 to about 0.06, about 0.04 to about 0.08, about 0.04 to about 0.1, about 0.06 to about 0.08, about 0.06 to about 0.1, or about 0.08 to about 0.1. In aspects, the polydispersity index of the population is about 0, about 0.002, about 0.004, about 0.006, about 0.008, about 0.01, about 0.02, about 0.04, about 0.06, about 0.08, or about 0.1.

### Microspheres

In aspects, the microsphere is a hydrogel microsphere. In aspects, the hydrogel microsphere disclosed herein comprises greater than about 30% water by weight, for instance, greater than about 35% water by weight, greater than about 40% water by weight, greater than about 45% water by weight, greater than about 50% water by weight, greater than about 55% water by weight, greater than about 60% water by weight, greater than about 65% water by weight, greater than about 70% water by weight, greater than about 75% water by weight, greater than about 80% water by weight, greater than about 85% water by weight, greater than about 90% water by weight, or greater than about 95% water by weight.

In aspects, the hydrogel microsphere disclosed herein has a water content of about 10 percent by weight to about 95 percent by weight, or about 20 percent by weight to about 95 percent by weight, or about 30 percent by weight to about 95 percent by weight, or about 40 percent by weight to about 95 percent by weight, or about 50 percent by weight to about 95 percent by weight, or about 60 percent by weight to about 95 percent by weight, or about 70 percent by weight to about 95 percent by weight, or about 80 percent by weight to about 95 percent by weight.

Without being bound by a theory, it is thought that the presence of pores in the microspheres disclosed herein increases the surface area per unit volume, which in turn promotes the interaction of synthetic cytotoxic cells with their target cells. Alternatively, porous hydrogel microspheres may exhibit different Young's modulus properties that result in improved cell interactions. As referred to herein, “porosity” may be used to refer to the percentage of void space within a particle (e.g., a hydrogel microsphere). When porogens are used (as discussed below), the porosity is the percentage of void space within the microsphere after removal of the porogens. In such a case, the porosity may comprise a plurality of micropores and a plurality of macropores, as described below. In aspects, the microspheres disclosed herein have a porosity of about 5% to about 95% of their volume. In aspects, the microspheres disclosed herein have a porosity of between about 80% and about 95% of their volume.

In aspects, the hydrogel microspheres disclosed herein comprise macropores and/or micropores. For instance, in aspects, the hydrogel microspheres disclosed herein comprise only micropores (and lack macropores) and are referred to herein as “smooth hydrogel microspheres”. In aspects, the hydrogel microspheres disclosed herein comprise micropores and macropores, and are referred to herein as “porous hydrogel microspheres”. In aspects, the porous hydrogel microspheres comprise a plurality of macropores at a concentration of at least ²0.25% v/v, at least 3.4% v/v, and/or at least 4.5% v/v. In aspects, the plurality of macropores comprise between about 2% and about 30% of a total number of pores of the porous hydrogel microsphere, the total number of pores of the porous hydrogel microsphere being a combination of the plurality of micropores and the plurality of macropores.

In aspects, the synthetic cytotoxic cells disclosed herein show an enhanced cytotoxic ability towards cells, such as cancer cells, as illustrated in the Examples. Without wishing to be bound by a theory, it is thought that such enhancement is promoted the presence of pores (e.g., macropores) in the hydrogel microspheres disclosed herein, which can result in (i) the provision of pores (e.g., macropores) as attachment sites for TRAIL or its active fragment to optimize its interactions with cells having TRAIL receptors, such as cancer cells; (ii) higher transportation rate of nutrients/water through pores; (iii) better absorption of water; (iv) maintenance of optimal ion nutrient gradient; and/or (v) maintenance of optimal osmotic pressure.

In aspects, the micropores disclosed herein have an average diameter of between about 1 nm and about 20 nm, or between about 2 nm and about 4 nm. In aspects, macropores of the present disclosure display an average diameter of about 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, or 200 nm.

In aspects, the macropores have an average diameter of between about 200 nm and about 2 μm. In aspects, macropores of the present disclosure display an average diameter of about 0.2 μm, 0.23 μm, 0.26 μm, 0.3 μm, 0.35 μm, 0.4 μm, 0.45 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, 2.6 μm, 2.7 μm, 2.8 μm, 2.9 μm, 3 μm, 3.1 μm, 3.2 μm, or 3.3 μm.

The microspheres disclosed herein may comprise a polymer. In aspects, the hydrogel microsphere is comprised of polymerized acrylamide and bis-acrylamide.

In aspects, the microsphere comprises a polymer material derived from one or more monomers. In aspects, the one or more monomers are selected from group consisting of: hydroxyethyl methacrylate, ethyl methacrylate, 2-hydroxyethyl methacrylate (HEMA), propylene glycol methacrylate, N-vinylpyrrolidone (NVP), methyl methacrylate, glycidyl methacrylate, glycerol methacrylate (GMA), glycol methacrylate, lactic acid, glycolic acid, poly(lactic-co-glycolic) acid (PLGA), ethylene glycol, fumaric acid, 2-hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, poly(ethylene glycol) methacrylate, methoxy-poly(ethylene glycol) methacrylate, methacrylic acid, sodium methacrylate, glycerol methacrylate, hydroxypropyl methacrylate, hydroxybutyl methacrylate, phenyl acrylate, phenyl methacrylate, benzyl acrylate, benzyl methacrylate, 2-phenylethyl acrylate, 2-phenylethyl methacrylate, 2-phenoxyethyl acrylate, 2-phenoxyethyl methacrylate, phenylthioethyl acrylate, phenylthioethyl methacrylate, 2,4,6-tribromophenyl acrylate, 2,4,6-tribromophenyl methacrylate, pentabromophenyl acrylate, pentabromophenyl methacrylate, pentachlorophenyl acrylate, pentachlorophenyl methacrylate, 2,3-dibromopropyl acrylate, 2,3-dibromopropyl methacrylate, 2-naphthyl acrylate, 2-naphthyl methacrylate, 4-methoxybenzyl acrylate, 4-methoxybenzyl methacrylate, 2-benzyloxyethyl acrylate, 2-benzyloxyethyl methacrylate, 4-chlorophenoxyethyl acrylate, 4-chlorophenoxyethyl methacrylate, 2-phenoxyethoxyethyl acrylate, 2-phenoxyethoxyethyl methacrylate, N-phenyl acrylamide, N-phenyl methacrylamide, N-benzyl acrylamide, N-benzyl methacrylamide, N,N-dibenzyl acrylamide, N,N-dibenzyl methacrylamide, N-diphenylmethyl acrylamide N-(4-methylphenyl)methyl acrylamide, N-1-naphthyl acrylamide, N-4-nitrophenyl acrylamide, N-(2-phenylethyl)acrylamide, N-triphenylmethyl acrylamide, N-(4-hydroxyphenyl)acrylamide, N,N-methylphenyl acrylamide, N,N-phenyl phenylethyl acrylamide, N-diphenylmethyl methacrylamide, N-(4-methyl phenyl)methyl methacrylamide, N-1-naphthyl methacrylamide, N-4-nitrophenyl methacrylamide, N-(2-phenylethyl)methacrylamide, N-triphenylmethyl methacrylamide, N-(4-hydroxyphenyl)methacrylamide, N,N-methylphenyl methacrylamide, N,N′-phenyl phenylethyl methacrylamide, acrylamide, bisacrylamide, streptavidin-acrylamide, N-vinylcarbazole, 4-vinylpyridine, 2-vinylpyridine, agar, agarose, alginic acid, alguronic acid, alpha glucan, amylopectin, amylose, arabinoxylan, beta-glucan, callose, capsulan, carrageenan polysaccharide, cellodextrin, cellulin, cellulose, chitin, chitosan, chrysolaminarin, curdlan, cyclodextrin, alpha-cyclodextrin, dextrin, dextran, ficoll, fructan, fucoidan, galactoglucomannan, galactomannan, galactosamino galactan, gellan gum, glucan, glucomannan, glucorunoxylan, glycocalyx, glycogen, hemicellulose, homopolysaccharide, hypromellose, icodextrin, inulin, kefiran, laminarin, lentinan, levan polysaccharide, lichenin, mannan, mixed-linkage glucan, paramylon, pectic acid, pectin, pentastarch, phytoglycogen, pleuran, polydextrose, polysaccharide peptide, porphyran, pullulan, schizophyllan, sinistrin, sizofiran, welan gum, xanthan gum, xylan, xyloglucan, and zymosan.

In aspects, the microsphere is biodegradable. In aspects, the microsphere comprises a polymer that is degradable. In aspects, the biodegradable polymer is a poly(esters) based on polylactide (PLA), polyglycolide (PGA), polycaprolactone (PCL), poly(lactic-co-glycolic) acid (PLGA), and their copolymers. In aspects, the polymer material comprises poly(lactic-co-glycolic acid) (PLGA). PLGA can be modified to have different ratios of lactic and glycolic acid, thus allowing for different dissolving rates. In aspects, the PLGA has a composition of poly(lactic acid):poly(glycolic acid) of between about 90:10 and about 10:90. In aspects, the ratio of lactic and glycolic acid is between 90:10 to 50:50. In aspects, the ratio of lactic and glycolic acid is about 85:15. In aspects, the ratio of lactic and glycolic acid is about 80:20. In aspects, the ratio of lactic and glycolic acid is about 75:25. In aspects, the ratio of lactic and glycolic acid is about 70:30. In aspects, the ratio of lactic and glycolic acid is about 65:35. In aspects, the ratio of lactic and glycolic acid is about 60:40. In aspects, the ratio of lactic and glycolic acid is about 55:45. In aspects, the ratio of lactic and glycolic acid is about 50:50. The PLGA microsphere allows for high motility of signals attached. Moreover, the PLGA microsphere is spherical in nature, allowing for stronger microsphere-to-cell contact. In aspects, the biodegradable polymer is used as a co-monomer, i.e., in a mixture of monomers. In aspects, the biodegradable polymer is a bifunctional monomer.

In aspects, the one or more monomers are selected from the group consisting of: agar, agarose, alginic acid, alguronic acid, alpha glucan, amylopectin, amylose, arabinoxylan, beta-glucan, callose, capsulan, carrageenan polysaccharide, cellodextrin, cellulin, cellulose, chitosan, chrysolaminarin, curdlan, cyclodextrin, alpha-cyclodextrin, dextrin, dextran, ficoll, fructan, fucoidan, galactoglucomannan, galactomannan, galactosamino galactan, gellan gum, glucan, glucomannan, glucorunoxylan, glycocalyx, glycogen, hemicellulose, homopolysaccharide, hypromellose, icodextrin, inulin, kefiran, laminarin, lentinan, levan polysaccharide, lichenin, mannan, mixed-linkage glucan, paramylon, pectic acid, pectin, pentastarch, phytoglycogen, pleuran, polydextrose, polysaccharide peptide, porphyran, pullulan, schizophyllan, sinistrin, sizofiran, welan gum, xanthan gum, xylan, xyloglucan, and zymosan, glycosaminoglycan (GAG). In aspects, the GAG is chondroitin sulfate, dermatan sulfate, keratin sulfate, heparin, heparin sulfate, or hyaluronic acid (also referred to in the art as hyaluron or hyaluronate).

In aspects, the one or more monomers comprise a protein or protein domain comprising non-natural amino acid. In aspects, the protein comprises natural amino acids. In aspects, self-assembling artificial proteins and proteins with non-natural amino acids (e.g., those incorporated into non-ribosomal peptides or synthetically introduced via synthetic approaches, see for example, Zhang et al. (2013). Current Opinion in Structural Biology 23, pp. 581-587, the disclosure of which is incorporated by reference in its entirety for all purposes), or protein domains thereof, can also be used as monomers. The range of non-natural (unnatural) amino acids that can be incorporated into such compositions is well known to those skilled in the art (Zhang et al. (2013). Current Opinion in Structural Biology 23, pp. 581-587; incorporated by reference in its entirety for all purposes). In aspects, the protein is a structural protein, or a domain thereof, for example, such as silk, elastin, titin or collagen, or a domain thereof. In aspects, the protein is an extracellular matrix (ECM) component (e.g., collagen, elastin, proteoglycan, fibrin, lysine, fibronectin). In aspects, the structural protein is collagen. In aspects, the collagen is collagen type I, collagen type II, or collagen type III, or a combination thereof. In aspects, the monomer comprises a proteoglycan. In aspects, the proteoglycan is decorin, biglycan, testican, bikunin, fibromodulin, lumican, or a domain thereof.

In aspects, the microsphere disclosed herein comprises a monofunctional monomer polymerized with a bifunctional monomer. A bifunctional monomer is any monomer that can polymerize with a monofunctional monomer of the disclosure to form a microsphere as described herein that further contains a second functional group that can participate in a second reaction, e.g., conjugation of a fluorophore, cell surface receptor (or domain thereof), or immune co-stimulatory biomolecule. Thus, in aspects, the microspheres disclosed herein comprise a fluorophore. In aspects, a bifunctional monomer is selected from the group consisting of allyl amine, allyl alcohol, allyl isothiocyanate, allyl chloride, and allyl maleimide. A bifunctional monomer can be a bifunctional acrylic monomer. Non-limiting examples of bifunctional acrylic monomers are N,N′-methylenebisacrylamide, N,N′-methylene bismethacrylamide, N,N′-ethylene bisacrylamide, N,N′-ethylene bismethacrylamide, N,N′-propylenebisacrylamide, and N,N′-(1,2-dihydroxyethylene) bisacrylamide. In aspects, the monomer is functionalized with acrylamide or acrylate. For example, in aspects, the polymerizable acrylamide functionalized biomolecule is an acrylamide or acrylate functionalized protein (for example, an acrylamide functionalized collagen or functionalized collagen domain), an acrylamide or acrylate functionalized peptide, or an acrylamide or acrylate functionalized monosaccharide, disaccharide, or polysaccharide.

In aspects, a microsphere provided herein comprises a polymerizable monofunctional monomer and is a monofunctional acrylic monomer. Non-limiting examples of monofunctional acrylic monomers for use herein are acrylamide; methacrylamide; N-alkylacrylamides such as N-ethylacrylamide, N-isopropylacrylamide or N-tert-butylacrylamide; N-alkylmethacrylamides such as N-ethylmethacrylamide or N-isopropylmethacrylamide; N,N-dialkylacrylamides such as N,N-dimethylacrylamide and N, N-diethyl-acrylamide; N-[(dialkylamino)alkyl]-acrylamides such as N-[3dimethylamino)-propyl]-acrylamide or N-[3-(diethylamino)propyl]-acrylamide; N-[(dialkylamino)alkyl]-methacrylamides such as N-[3-dimethylamino)propyl]methacrylamide or N-[3-(diethylamino) propyl]methacrylamide; (dialkylamino)alkyl acrylates such as 2-(dimethylamino)ethyl acrylate, 2-(dimethylamino)propyl acrylate, or 2-(diethylamino)ethyl acrylates; and (dialkylamino) alkyl methacrylates such as 2-(dimethylamino) ethyl methacrylate.

In aspects, the monomer is ethylene glycol dimethacrylate (EGDMA), 2-hydroxyethyl methacrylate (HEMA), methyl methacrylate (MMA), methacryloxymethyltrimethylsilane (TMS-MA), N-vinyl-2-pyrrolidon (N-VP), styrene, or a combination thereof. Higher order branched chain and linear co-monomers can be substituted in the polymer mix to adjust the refractive index while maintaining polymer density, as described in U.S. Pat. No. 6,657,030, which is incorporated herein by reference in its entirety for all purposes. In aspects, a microsphere comprises a molecule that modulates the optical properties of the microsphere.

Examples of various monomers and cross-linking chemistries available for use with the present disclosure are provided in the Thermo Scientific Crosslinking Technical Handbook entitled “Easy molecular bonding crosslinking technology” (available at tools.lifetechnologies.com/content/sfs/brochures/1602163-Crosslinking-Reagents-Handbook.pdf), the disclosure of which is incorporated by reference in its entirety for all purposes. An additional range of monomers that can be incorporated are known in the art, see, for example, as disclosed in Shastri (2003). Current Pharmaceutical Biotechnology 4, pp. 331-337, incorporated by reference herein in its entirety for all purposes. Other monomers are provided in de Moraes Porto (2012). Polymer Biocompatibility, Polymerization, Dr. Ailton De Souza Gomes (Ed.), ISBN: 978-953-51-0745-3; InTech, DOI: 10.5772/47786; Heller et al. (2010). Journal of Polymer Science Part A: Polymer Chemistry 49, pp. 650-661; Final Report for Biocompatible Materials (2004), The Board of the Biocompatible Materials and the Molecular Engineering in Polymer Science programmes, ISBN 91-631-4985-0, the disclosure of each of which is incorporated herein by reference in its entirety for all purposes.

### Methods of Preparing Microspheres

In aspects, the present disclosure provides methods of producing microspheres, comprising a dispersed monomer phase and a continuous suspension phase, such as oil. In aspects, the monomeric material (monomer) is polymerized to form a homopolymer. In aspects, copolymers of different monomeric units (i.e., co-monomers) are synthesized and used in the methods disclosed herein. In aspects, the microsphere is synthesized in the presence of a crosslinker. In aspects, the microsphere is synthesized in the presence of a polymerization initiator.

The amount of monomer can be varied, for example to obtain a particular optical property that is substantially similar to that of a cell. In aspects, the monomeric component(s) (i.e., monomer, co-monomer, bifunctional monomer, or a combination thereof, for example, bis/acrylamide in various crosslinking ratios, allyl amine, or other co-monomers which provide chemical functionality for secondary labeling/conjugation, or alginate) is present at about 10 percent by weight to about 95 percent weight of the microsphere. In aspects, the monomeric component(s) is present at about 15 percent by weight to about 90 percent weight of the microsphere, or about 20 percent by weight to about 90 percent weight of the microsphere.

The microspheres of the present disclosure can be further modified by varying the size of the microsphere produced, thus mimicking different types or states of cells. Size of the microsphere can be controlled by flow rates and/or pressure of the aqueous and oil phase during the microfluidic droplet generation process. Average diameters of the microspheres can be measured, for example, by light scattering techniques. Further details on methods of preparing microspheres are provided in PCT/US2024/018187 and US 2016/0258856, which are both incorporated herein by reference in their entireties for all purposes.

In aspects, a porogen is present mixed with the monomer phase. In general, any material that a) can phase separate (is not miscible) with the matrix and b) does not get incorporated into/tethered to the matrix and can be removed after formation of the matrix can be used as a porogen for the synthesis of porous hydrogel microspheres. In aspects, porogens may be immiscible within the monomer, and thus may be said to form a further dispersed phase within the monomer phase (i.e., where porogen may be considered the dispersed phase and the monomer phase would be considered a continuous phase). These embodiments could be described as an emulsion within an emulsion. For the purposes of this disclosure however, the monomer phase is referred to as the dispersed phase, regardless of whether it also includes porogens. The continuous phase refers to the suspension (e.g., oil) phase. In aspects, the monomer to be polymerized may be within a first phase and the porogen may be within a second phase.

In aspects, the porogen may be one or more of a porogen polymer, a water-soluble polymer, a salt, carbon black, a biodegradable polymer, a degradable polymer, seaweed polysaccharides, and a paraffin wax. In aspects, the porogen polymer comprises one or more of polyethylene glycol, poly(vinylpyrrolidone), polyvinyl alcohol, and any combination thereof. For instance, the porogen polymer may include polymers that are water-soluble but also gel matrix polymer immiscible may also be used.

In aspects, the porogen polymer can have a linear, branched, hyperbranched, or a bottlebrush structure. In aspects, the porogen polymer may comprise polymeric particles that become water-soluble after a stimulus is applied. For example, particles with a degradable crosslinker (e.g. N,N′-Bis(acryloyl)cystamine) can be embedded into particles and then degraded with a cleaving agent. (e.g. reducing agent for N,N′-Bis(acryloyl)cystamine). In aspects, creating a porous structure increases the surface area of the microsphere. The percentage of the material forming the microsphere, the molecular weight of the porogen and the % concentration of the porogen added can be adjusted to achieve a desired porosity.

In aspects, polyethylene glycol (PEG), which is water-soluble, may be used as the porogen. PEG is immiscible with polyacrylamide. In aspects, inert, linear PEG polymer can be introduced as a porogen into the aqueous or water phase of our microfluidic synthesis of microspheres. During the curing process, the linear PEG polymers, immiscible with the gel matrix polymer (poly acrylamide in this case), become phase separated with the gel matrix and form its own domains, spatially excluding polyacrylamide microspheres. After synthesis, the beads are washed with water where the PEG polymers are removed from the matrix. This leaves hollow pores within the microspheres.

Table 6 shows previously characterized hydrodynamic radius of various PEG polymer molecular weights, and thus the minimum implied pore size introduced by their inclusion in microspheres, as an example of a porogen polymer used within the microspheres of the present disclosure.

TABLE 6

| Molecular Weight | Hydrodynamic Radius |
|---|---|
| (kDa) | (nm) |
| PEG 200 | 0.49 |
| PEG 400 | 0.65 |
| PEG 1000 | 0.93 |
| PEG 4000 | 1.60 |
| PEG 10,000 | 2.29 |
| PEG 20,000 | 3.01 |
| PEG 40,000 | 3.95 |

In aspects, the PEG concentration within the dispersed phase may be between about 1% w/v and about 99% w/v. For instance, the PEG concentration may be at least about 1%, at least about 2%, at least about 4%, at least about 6%, at least about 8%, at least about 10%, at least about 12%, at least about 14%, at least about 16%, at least about 18%, at least about 20%, at least about 25%, at least about 30%, at least about 35%, or at least about 40% w/v, including all ranges and subranges therebetween. In aspects, the PEG concentration introduced during preparation of the microspheres may be about 9% w/v. In aspects, the PEG concentration introduced during preparation of the microspheres may be about 2.25%, about 3.4%, or about 4.5% w/v, including all ranges and subranges therebetween. In aspects, the PEG concentration within the dispersed phase may be between about 1% v/v and about 99% v/v. In aspects, the PEG solution comprises a variable concentration of PEG 8000.

In aspects, the molecular weight of PEG is about 0.1 kDa to about 100 kDa. In aspects, the molecular weight of PEG is at least about 0.1 kDa. In aspects, the molecular weight of PEG is at most about 100 kDa. In aspects, the molecular weight of PEG is about 0.1 kDa to about 0.5 kDa, about 0.1 kDa to about 1 kDa, about 0.1 kDa to about 3 kDa, about 0.1 kDa to about 6 kDa, about 0.1 kDa to about 9 kDa, about 0.1 kDa to about 10 kDa, about 0.1 kDa to about 20 kDa, about 0.1 kDa to about 50 kDa, about 0.1 kDa to about 100 kDa, about 0.5 kDa to about 1 kDa, about 0.5 kDa to about 3 kDa, about 0.5 kDa to about 6 kDa, about 0.5 kDa to about 9 kDa, about 0.5 kDa to about 10 kDa, about 0.5 kDa to about 20 kDa, about 0.5 kDa to about 50 kDa, about 0.5 kDa to about 100 kDa, about 1 kDa to about 3 kDa, about 1 kDa to about 6 kDa, about 1 kDa to about 9 kDa, about 1 kDa to about 10 kDa, about 1 kDa to about 20 kDa, about 1 kDa to about 50 kDa, about 1 kDa to about 100 kDa, about 3 kDa to about 6 kDa, about 3 kDa to about 9 kDa, about 3 kDa to about 10 kDa, about 3 kDa to about 20 kDa, about 3 kDa to about 50 kDa, about 3 kDa to about 100 kDa, about 6 kDa to about 9 kDa, about 6 kDa to about 10 kDa, about 6 kDa to about 20 kDa, about 6 kDa to about 50 kDa, about 6 kDa to about 100 kDa, about 9 kDa to about 10 kDa, about 9 kDa to about 20 kDa, about 9 kDa to about 50 kDa, about 9 kDa to about 100 kDa, about 10 kDa to about 20 kDa, about 10 kDa to about 50 kDa, about 10 kDa to about 100 kDa, about 20 kDa to about 50 kDa, about 20 kDa to about 100 kDa, or about 50 kDa to about 100 kDa. In aspects, the molecular weight of PEG is about 0.1 kDa, about 0.5 kDa, about 1 kDa, about 3 kDa, about 6 kDa, about 8 kDa, about 9 kDa, about 10 kDa, about 20 kDa, about 50 kDa, or about 100 kDa. In aspects, the PEG is PEG 8000.

In general, any form of polymerization chemistry/methods known by those skilled in the art can be employed to form polymers. In aspects, polymerization can be catalyzed by ultraviolet light-induced radical formation and reaction progression. In aspects, polymerization of a monomer is initiated by a persulfate or an equivalent initiator that catalyzes radical formation. The persulfate can be any water-soluble persulfate. Non-limiting examples of water-soluble persulfates are ammonium persulfate and alkali metal persulfates. Alkali metals include lithium, sodium, and potassium. In aspects, the persulfate is ammonium persulfate or potassium persulfate. In aspects, polymerization of the monomer provided herein is initiated by ammonium persulfate. In aspects, suspension polymerization, emulsion polymerization, or precipitation polymerization may be used.

Polymerization of a monomer can be accelerated by an accelerant which can catalyze the formation of polymerization-labile chemical side groups. In aspects, the accelerant is a tertiary amine. The tertiary amine can be any water-soluble tertiary amine. In aspects, an accelerant is used in the polymerization reaction and is 3-(dimethylamino) propionitrile, or N,N,N′,N′tetramethylethylenediamine (TEMED). In aspects, an accelerant is used in the polymerization reaction and is azobis(isobutyronitrile) (AIBN).

As discussed above, the microspheres disclosed herein in aspects are produced as particles by polymerizing droplets. Microfluidic methods of producing a plurality of droplets, including fluidic and rigidified droplets, are known to those of ordinary skill in the art, and described in US Patent Publication No. 2011/0218123 and U.S. Pat. No. 7,294,503, each incorporated herein by reference in its entirety for all purposes. Such methods provide for a plurality of droplets containing a first fluid (e.g., dispersed phase) and being substantially surrounded by a second fluid (e.g., a continuous phase), where the first fluid and the second fluid are substantially immiscible (e.g., droplets containing an aqueous-based liquid being substantially surrounded by an oil-based liquid). Non-limiting examples of microfluidic systems that may be used with the present disclosure are disclosed in U.S. Patent Application Publication No. 2006/0163385; U.S. Patent Application Publication No. 2005/0172476; U.S. Patent Application Publication No. 2007/000342; International Patent Application Publication No. WO 2006/096571; U.S. Patent Application Publication No. 2007/0054119; U.S. Pat. No. 7,776,927; and International Patent Application Publication No. WO 2006/078841, each incorporated herein by reference in its entirety for all purposes.

### TNF-Related Apoptosis-Inducing Ligand (TRAIL)

In aspects, the TRAIL protein or active fragment thereof comprises an amino acid sequence of the human full length TRAIL protein. In aspects, the TRAIL protein or active fragment thereof comprises an amino acid sequence that is at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 1. In aspects, the TRAIL or active fragment thereof comprises or consists of the amino acid sequence of SEQ ID NO: 1.

As used herein, an “active fragment of TRAIL” comprises a region of the TRAIL protein that is capable of binding to one or more TRAIL receptors, such as DR4 and/or DR5. In aspects, the active fragment of TRAIL does not comprise the transmembrane domain of the TRAIL protein and/or the N-terminal intracellular domain of the TRAIL protein. In aspects, the transmembrane domain of the TRAIL protein comprises amino acid residues 12 to 38 of SEQ ID NO: 1, or a sequence with at least 99%, at least 95%, at least 90%, about 85%, or at least 80% identity thereto. In aspects, the transmembrane domain of the TRAIL protein comprises amino acid residues 12 to 38 of SEQ ID NO: 1. In aspects, the N-terminal domain of the TRAIL protein comprises amino acid resides 1 to 11, 1 to 12, 1 to 13, 1 to 14, or 1 to 15 of the TRAIL protein. In aspects, the N-terminal domain of the TRAIL protein comprises amino acid resides 1 to 11 of the TRAIL protein.

In aspects, the active fragment of TRAIL comprises the C-terminal extracellular region, which comprises a TNF homology domain (THD) and an extracellular stalk. In aspects, the active fragment of TRAIL comprises a stretch of about 50 to about 242 amino acids of a region comprising the amino acid residues 39 to 281 of SEQ ID NO: 1, or an amino acid sequence having at least 80%, at least 85%, at least 90%, at least 95%, at least 96%, at least 97%, at least 98%, or at least 99% identity thereto. In aspects, the active fragment of TRAIL comprises amino acid residues 39 to 281 of an amino acid sequence having at least 80%, at least 85%, at least 90%, at least 95%, at least 96%, at least 97%, at least 98%, at least 99%, or 100% identity to SEQ ID NO: 1.

In aspects, the active fragment of TRAIL comprises the TNF family domain of the TRAIL protein. In aspects, the active fragment of TRAIL comprises a stretch of about 50 to about 157 amino acids of a region comprising the amino acid residues 123 to 280 of SEQ ID NO: 1, or an amino acid sequence having at least 80%, at least 85%, at least 90%, at least 95%, at least 96%, at least 97%, at least 98%, or at least 99% identity thereto. In aspects, the active fragment of TRAIL comprises amino acid residues 123 to 280 of an amino acid sequence having at least 80%, at least 85%, at least 90%, at least 95%, at least 96%, at least 97%, at least 98%, at least 99%, or 100% identity to SEQ ID NO: 1.

In aspects, the active fragment of TRAIL comprises a stretch of about 50 to about 167 amino acids of a region comprising the amino acid residues 114 to 281 of SEQ ID NO: 1, or an amino acid sequence having at least 80%, at least 85%, at least 90%, at least 95%, at least 96%, at least 97%, at least 98%, or at least 99% identity thereto. In aspects the active fragment of TRAIL comprises amino acid residues 114 to 281 of an amino acid sequence having at least 80%, at least 85%, at least 90%, at least 95%, at least 96%, at least 97%, at least 98%, at least 99%, or 100% identity to SEQ ID NO: 1. In aspects, the active fragment of TRAIL comprises an amino acid sequence having at least 80%, at least 85%, at least 90%, at least 95%, at least 96%, at least 97%, at least 98%, at least 99%, or 100% identity to SEQ ID NO: 2.

In aspects, the TRAIL protein or active fragment thereof comprises an amino acid sequence having at least 80%, at least 85%, at least 90%, at least 95%, at least 96%, at least 97%, at least 98%, at least 99% identity, or 100% identity to any one of the TRAIL proteins listed below in Table 7.

TABLE 7

| UniPort Entry | Organism |
|---|---|
| A0A096MXB8 | Papio anubis (Olive baboon) |
| A0A0D9RK57 | Chlorocebus sabaeus (Green monkey) |
| | (Cercopithecus sabaeus) |
| A0A0U5J5G6 | Pan troglodytes (Chimpanzee) |
| A0A2I3HA28 | Nomascus leucogenys (Northern white-cheeked |
| | gibbon) (Hylobates leucogenys) |
| A0A2J8TIM1 | Pongo abelii (Sumatran orangutan) |
| | (Pongo pygmaeus abelii) |
| A0A2K5P7D2 | Cercocebus atys (Sooty mangabey) |
| | (Cercocebus torquatus atys) |
| A0A2K5RPW0 | Cebus imitator (Panamanian white-faced |
| | capuchin) (Cebus capucinus imitator) |
| A0A2K5YDQ1 | Mandrillus leucophaeus (Drill) |
| | (Papio leucophaeus) |
| A0A2K6D6E1 | Macaca nemestrina (Pig-tailed macaque) |
| A0A2K6NUD9 | Rhinopithecus roxellana (Golden snub-nosed |
| | monkey) (Pygathrix roxellana) |
| A0A2K6UF26 | Saimiri boliviensis boliviensis (Bolivian squirrel monkey) |
| A0A2R9C2U3 | Pan paniscus (Pygmy chimpanzee) (Bonobo) |
| F6S9W7 | Macaca mulatta (Rhesus macaque) |
| F7E747 | Callithrix jacchus (White-tufted-ear marmoset) |
| G3RF33 | Gorilla gorilla gorilla (Western lowland gorilla) |
| I7GIW0 | Macaca fascicularis (Crab-eating macaque) |
| | (Cynomolgus monkey) |
| A0A2K5HK60 | Colobus angolensis palliatus (Peters' Angolan colobus) |
| A0A2K6UF21 | Saimiri boliviensis boliviensis (Bolivian squirrel monkey) |
| A0A6J3G868 | Sapajus apella (Brown-capped capuchin) (Cebus apella) |
| A0A803KKU8 | Pan troglodytes (Chimpanzee) |
| A0A8C9LVQ6 | Piliocolobus tephrosceles (Ugandan red Colobus) |
| A0A8D2GCR1 | Theropithecus gelada (Gelada baboon) |
| G2HF74 | Pan troglodytes (Chimpanzee) |

In aspects, the TRAIL protein or active fragment thereof comprises one or more amino acid modifications. Amino acid modifications may be amino acid substitutions, amino acid deletions, and/or amino acid insertions. Amino acid substitutions may be conservative amino acid substitutions or non-conservative amino acid substitutions. A conservative replacement (also called a conservative mutation, a conservative substitution, or a conservative variation) is an amino acid replacement in a protein that changes a given amino acid to a different amino acid with similar biochemical properties (e.g., charge, hydrophobicity, and size). As used herein, “conservative variations” refer to the replacement of an amino acid residue by another, biologically similar residue. Examples of conservative variations include the substitution of one hydrophobic residue such as isoleucine, valine, leucine, or methionine for another, or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to praline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

In aspects, the TRAIL or active fragment thereof disclosed herein are chemically modified, for example, by the covalent attachment of any type of molecule to the TRAIL or active fragment thereof. Exemplary non-limiting modifications include glycosylation, acetylation, pegylation, phosphorylation, amidation, derivatization by known protecting/blocking groups, proteolytic cleavage, linkage to a cellular ligand or other protein, etc. Additionally, the TRAIL or active fragment thereof may contain one or more non-classical amino acids. In aspects, a polyethylene glycol (PEG) is conjugated to TRAIL or active fragment thereof. In aspects, the molecular weight of PEG is about 0.1 kDa to about 100 kDa. In aspects, the molecular weight of PEG is at least about 0.1 kDa. In aspects, the PEG is PEG 8000.

In aspects, the TRAIL protein or active fragment thereof is present as a monomer on the surface of the microsphere. In aspects, the TRAIL protein or active fragment thereof is present as a trimer on the surface of the microsphere. In aspects, within the multimer or the trimer, each of the TRAIL protein or active fragment thereof is linked to other TRAIL protein(s) or active fragment(s) thereof via non-covalent links. In aspects, the non-covalent link is the non-covalent interactions with a metal ion. In aspects, the metal ion is a Zn ion.

In aspects, within the multimer or the trimer, each of the TRAIL protein or active fragment thereof is linked to other TRAIL protein(s) or active fragment(s) thereof via covalent link within the multimer. In aspects, the multimer comprises at least a first TRAIL or active fragment thereof, and a second TRAIL or active fragment thereof, wherein a linker attaches the first TRAIL or active fragment thereof, to the second TRAIL or active fragment thereof. In aspects, the linker comprises one or more amino acids. In aspects, the linker comprises one or more serine (S) residues and one or more glycine (G) residues. In aspects, the linker is a (GxS)n linker, wherein x can be an integer between 1 and 10, and n can be an integer between 2 and 20. In aspects, the TRAIL protein or active fragment thereof is linked to an Fc domain. In aspects, the TRAIL protein or active fragment thereof is biotinylated.

### Surface-Attachment of TRAIL or Active Fragment Thereof on Microspheres

In aspects, the synthetic cytotoxic cells disclosed herein show an enhanced cytotoxic ability towards cells, such as cancer cells, as illustrated in the Examples. Without wishing to be bound by a theory, it is thought that such enhancement is promoted by the attachment of the TRAIL protein or active fragment thereof to the surface of the microspheres, which facilitates its interaction with its receptor (e.g., a TRAIL receptor, such as DR4 or DR5) on cells, such as cancer cells. In some embodiments, the present invention utilizes hydrogels as synthetic cytotoxic cell mimics, rather than merely delivery vehicles for TRAIL. That is, it is hypothesized that the improved activity of the present invention, may in part, relate to the attachment of TRAIL to a fixed cell mimic.

In aspects, the TRAIL protein or active fragment thereof is covalently attached to the microsphere. Non-limiting examples of covalent linkers include disulfide linkers, ester linkers, amine linkers, thiol linkers, and carbonyl linkers.

In aspects, the TRAIL protein or active fragment thereof is non-covalently attached to the microsphere. Non-limiting examples of non-covalent linkers include streptavidin-biotin, neutravidin-biotin, and affinity tags such as His-tag, GST-tag, Halo-tag, and SNAP-tag. In aspects, the TRAIL protein or active fragment thereof may be attached to the microsphere via a free amine, free carboxyl, and/or free hydroxyl group present on the surface of the microsphere. Functionalization of the microsphere with a cell surface molecule can also occur through a linker, such as by a streptavidin/biotin conjugate, a biotin/streptavidin conjugate, a streptavidin/biotin/streptavidin conjugate, and/or a biotin/streptavidin/biotin conjugate. For instance, when the microsphere comprises acrylamide, a streptavidin-biotin linkage can be exploited to attach the TRAIL or active fragment thereof to the surface of the microspheres.

Other known binding/linkage methods can be used without departing from the spirit of the present disclosure. In aspects, the microsphere is capable of attaching to an immune response biomolecule, such as a TRAIL receptor, via a linker. In aspects, the linker comprises a polypeptide, a ligand (e.g., a TRAIL protein or active fragment thereof), or an antibody. In aspects, the immune response biomolecule (e.g., a TRAIL receptor) is located on the surface of a cell.

In aspects, the synthetic cytotoxic cell comprises between about 1 and about 100,000,000 copies of the TRAIL ligand or its active fragment. In aspects, the synthetic cytotoxic cell is approximately the same size as the target cell and comprises between about 500 and 100,000,000 copies of the TRAIL ligand or its active fragment. In aspects, the synthetic cytotoxic cell is approximately about 5 μm to about 200 μm and comprises between about 500 and 100,000,000 copies of the TRAIL ligand or its active fragment. In aspects, the synthetic cytotoxic cell has a diameter of at least 5 μm. In aspects, the synthetic cytotoxic cell comprises at least the same number of the TRAIL ligand or its active fragment as the number of TRAIL receptors on the target cell. In aspects, the synthetic cytotoxic cell comprises more of the TRAIL ligand or its active fragment than the number of TRAIL receptors on the target cell. In aspects, the synthetic cytotoxic cell comprises at least 1, at least 10, at least 100, at least 1,000, at least 10,000, at least 100,000, at least 1,000,000, at least 10,000,000, or at least 100,000,000 copies of the TRAIL ligand or its active fragment.

### Biological Complexes

As noted herein, the synthetic cytotoxic cells of the present disclosure are capable of tethering to biological cells, thereby inducing apoptosis. In some embodiments the biological cells express DR4 and/or DR5. Therefore, in aspects, the present disclosure teaches a biological complex, comprising: a) a hydrogel microsphere with a surface; and b) a biological cell comprising a TRAIL receptor selected from the group consisting of DR4 and DR5; wherein the biological cell is tethered to the hydrogel microsphere via the TRAIL receptor. In aspects, the present disclosure also teaches a synthetic cytotoxic cell, comprising: a) a hydrogel microsphere with a surface; and b) a TRAIL receptor selected from the group consisting of DR4 and DR5; wherein the TRAIL receptor is tethered to the surface of the hydrogel microsphere.

In aspects, the TRAIL receptor(s) DR4 and/or DR5 are tethered to the cytotoxic cell via non-covalent linkers. Non-limiting examples of non-covalent linkers include streptavidin-biotin, neutravidin-biotin, and affinity tags such as His-tag, GST-tag, Halo-tag, SNAP-tag, ligands or antibodies against DR4 and/or DR5, and a TRAIL protein, or active fragment thereof.

In aspects, the TRAIL receptor(s) DR4 and/or DR5 are tethered to the cytotoxic cell via covalent linkers. Non-limiting examples of covalent linkers include disulfide linkers, ester linkers, amine linkers, thiol linkers, and carbonyl linkers.

In aspects, the DR4 and/or DR5 TRAIL receptor(s) that are tethered to the synthetic cytotoxic cells are simultaneously tethered to the biological cell, thereby also transiently tethering the biological cell to the synthetic cytotoxic cell.

In aspects, the present disclosure teaches methods of producing biological complexes or synthetic cytotoxic cells tethered to DR4 and/or DR5, said method comprising the steps of contacting a synthetic cytotoxic cell of the present disclosure with a biological cell expressing DR4 and/or DR5, wherein the synthetic cytotoxic cell is tethered to the biological cell via the DR4 and/or DR5 TRAIL receptors using a covalent, or non-covalent linker. In some embodiments, the non-covalent linker is a TRAIL protein, or an active fragment thereof.

### Compositions

Disclosed herein are compositions, such as pharmaceutical compositions, comprising the synthetic cytotoxic cells disclosed herein. In aspects, the pharmaceutical composition further comprises a pharmaceutical acceptable carrier.

Pharmaceutically acceptable carrier, diluent or excipient includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals. Exemplary pharmaceutically acceptable carriers include, but are not limited to, sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter; waxes; animal and vegetable fats; paraffins; silicones; bentonites; silicic acid; zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

Pharmaceutically acceptable salt includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

Also disclosed herein are kits for carrying out a method described herein. In aspects, a kit comprises the synthetic cytotoxic cells disclosed herein. In aspects, a kit further comprises instructions for using the components of the kit to practice the methods of the present disclosure.

The disclosure further provides conjugates comprising a cell and any of the synthetic cytotoxic cells disclosed herein. The disclosure also provides cells, wherein the cell is conjugated to any one of the synthetic cytotoxic cells disclosed herein. The disclosure further provides mixtures of (i) a cell, and a (ii) any of the synthetic cytotoxic cells disclosed herein.

In aspects, the cell and the synthetic cytotoxic cell are non-covalently conjugated. In aspects, the cell expresses one or both of: (a) a TRAIL receptor DR4 and (b) a TRAIL receptor DR5. In aspects, the cell expresses a TRAIL receptor DR4. In aspects, the TRAIL receptor DR4 comprises an amino acid sequence having at least 80%, at least 85%, at least 90%, at least 95%, at least 96%, at least 97%, at least 98%, at least 99%, or 100% identity to SEQ ID NO: 3. In aspects, the cell expresses a TRAIL receptor DR5, or an antigen binding fragment thereof. In aspects, the TRAIL receptor DR5 comprises an amino acid sequence having at least 80%, at least 85%, at least 90%, at least 95%, at least 96%, at least 97%, at least 98%, at least 99%, or 100% identity to SEQ ID NO: 4. In aspects, the cell is conjugated to the synthetic cytotoxic cell via an interaction between: (i) the TRAIL or active fragment thereof and DR4; (ii) the TRAIL or active fragment thereof and DR5; or (iii) both (i) and (ii). In aspects, the interaction between the TRAIL or active fragment thereof, and one or both of DR4 and DR5 results in the induction of apoptosis and/or necrosis of the cell. In aspects, the cell is a cancer cell.

The disclosure also provides synthetic cytotoxic cells, comprising a) a hydrogel microsphere with a surface; and b) a TRAIL receptor selected from the group consisting of DR4 and DR5; wherein the TRAIL receptor is tethered to the surface of the hydrogel microsphere. The disclosure also provides biological complexes, comprising a) a hydrogel microsphere with a surface; and b) a biological cell comprising a TRAIL receptor selected from the group consisting of DR4 and DR5; wherein the biological cell is tethered to the hydrogel microsphere via the TRAIL receptor. In aspects, the TRAIL receptor is tethered to the surface of the hydrogel microsphere via a linker. In aspects, the linker comprises a biotin/streptavidin complex. In aspects, the TRAIL receptor is covalently linked to the linker. In aspects, the TRAIL receptor is non-covalently linked to the linker. In aspects, the TRAIL receptor is tethered to the surface of the hydrogel microsphere via an interaction between the TRAIL or the active fragment thereof, and the TRAIL receptor.

### Methods of Use

The disclosure provides methods of inducing apoptosis and/or necrosis of a target cell, comprising contacting the target cell with the synthetic cytotoxic cell or the composition disclosed herein, thereby inducing apoptosis and/or necrosis of the target cell. In aspects, the contacting of the target cell is performed in vitro, ex vivo, or in vivo. In aspects, the contacting of the target cell is performed in vivo. In aspects, the method comprises administering the synthetic cytotoxic cell or the composition to a subject in need thereof.

The disclosure provides methods of inducing apoptosis and/or necrosis of a target cell in a subject in need thereof, comprising administering the synthetic cytotoxic cell or the composition disclosed herein to the subject, thereby inducing apoptosis and/or necrosis of the target cell in the subject. In aspects, the administering results in contacting of the target cell with the synthetic cytotoxic cell in vivo. In aspects, the target cell is a cancer cell.

The disclosure provides methods of treating a cancer in a subject in need thereof, comprising administering the synthetic cytotoxic cell or the composition disclosed herein to the subject. In aspects, the method comprises inducing apoptosis and/or necrosis of a target cell in the subject. In aspects, the subject is a human subject.

In aspects, the target cell expresses one or both of: (a) a TRAIL receptor DR4; and (b) a TRAIL receptor DR5. In aspects, the target cell expresses a TRAIL receptor DR4. In aspects, the target cell expresses a TRAIL receptor DR5. In aspects, the method results in an interaction between: (i) the TNF-related apoptosis-inducing ligand (TRAIL), or active fragment thereof and a TRAIL receptor DR4; (ii) the TNF-related apoptosis-inducing ligand (TRAIL), or active fragment thereof and a TRAIL receptor DR5; (iii) both (i) and (ii).

In aspects, the cancer is blood cancer. In aspects, the cancer is a solid tumor. For example, cancers that may be treated using the compositions and methods disclosed herein include, but are not limited to, leukemia (e.g., chronic lymphocytic and acute), lymphoma, myeloma, carcinoma, sarcoma (e.g., Ewing and chondrosarcoma), or brain and spinal cord cancer. In aspects, the cancer is chronic lymphocytic leukemia (CLL), B cell acute lymphocytic leukemia (B-ALL), acute lymphoblastic leukemia (ALL), acute myeloid leukemia (AML), non-Hodgkin's lymphoma (NHL), diffuse large cell lymphoma (DLCL), diffuse large B cell lymphoma (DLBCL), Hodgkin's lymphoma, multiple myeloma, renal cell carcinoma (RCC), hepatocellular carcinoma, melanoma, mesothelioma, colorectal cancer, bladder cancer, breast cancer (such as triple negative breast cancer), colorectal cancer, ovarian cancer, prostate cancer, lung cancer, esophageal cancer, pancreatic cancer, head and neck cancer, liver cancer, cervical cancer, breast cancer, astrocytoma, medulloblastoma, neuroblastoma, non-small cell lung cancer, peritoneal malignancies, peritoneal carcinomatosis, solid tumor, malignant pleura mesothelioma, gastric cancer, urothelial cancer, cholangiocarcinoma, and hepatocellular carcinoma. In aspects, the cancer is insensitive or resistant to the standard of care. In aspects, the cancer is sensitive to TRAIL-induced apoptosis.

In aspects, the method results in a proportion of apoptotic and/or necrotic target cells that is higher than the proportion of apoptotic and/or necrotic target cells resulting from contacting the target cell with a soluble form of TRAIL or active fragment thereof.

In aspects, the method results in a proportion of apoptotic and/or necrotic target cells that is higher than the proportion of apoptotic and/or necrotic target cells resulting from contacting the target cell with a comparator microsphere, wherein the comparator microsphere comprises a non-hydrogel microsphere and the TRAIL or active fragment thereof, wherein the TRAIL or active fragment thereof is attached to the surface of the non-hydrogel microsphere.

In aspects, the method results in a proportion of apoptotic and/or necrotic target cells that is higher than the proportion of apoptotic and/or necrotic target cells resulting from contacting the target cell with a comparator microsphere, wherein the comparator microsphere comprises a microsphere and TRAIL or active fragment thereof, wherein the TRAIL or active fragment thereof is encapsulated by the microsphere. In aspects, the comparator microsphere comprises a microsphere comprised of poly (lactic-co-glycolic acid) that encapsulates TRAIL or active fragment thereof.

The disclosure provides methods of treating a disease (e.g., a cancer) in a subject in need thereof, comprising administering the synthetic cytotoxic cell or the composition disclosed herein comprising a porous hydrogel microsphere to the subject. In aspects, the method of administering the synthetic cytotoxic cell or the composition disclosed herein comprising a porous hydrogel microsphere results in a proportion of apoptotic and/or necrotic target cells that is higher than the proportion of apoptotic and/or necrotic target cells resulting from contacting the target cell with a comparator microsphere, wherein the comparator microsphere comprises a smooth porous microsphere and a TNF-related apoptosis-inducing ligand (TRAIL), or active fragment thereof, wherein the TRAIL or active fragment thereof is attached to the surface of the smooth porous microsphere.

In aspects, the method of administering the synthetic cytotoxic cell or the composition disclosed herein comprising a porous hydrogel microsphere, results in a proportion of early apoptotic target cells that is higher than the proportion of early or late apoptotic target cells resulting from contacting the target cell with a soluble form of TRAIL or active fragment thereof.

In aspects, the method of administering the synthetic cytotoxic cell or the composition disclosed herein comprising a porous hydrogel microsphere, results in a proportion of early apoptotic target cells that is higher than the proportion of early or late apoptotic target cells resulting from contacting the target cell with a comparator microsphere, wherein the comparator microsphere comprises a non-hydrogel microsphere and a TNF-related apoptosis-inducing ligand (TRAIL), or active fragment thereof, wherein the TRAIL or active fragment thereof is attached to the surface of the non-hydrogel microsphere.

In aspects, the method of administering the synthetic cytotoxic cell or the composition disclosed herein comprising a porous hydrogel microsphere, results in a proportion of early apoptotic target cells that is higher than the proportion of early or late apoptotic target cells resulting from contacting the target cell with a comparator microsphere, wherein the comparator microsphere comprises a TNF-related apoptosis-inducing ligand (TRAIL), or active fragment thereof, wherein the TRAIL or active fragment thereof is encapsulated by the microsphere, optionally, wherein the microsphere is comprised of poly (lactic-co-glycolic acid).

In aspects, the method of administering the synthetic cytotoxic cell or the composition disclosed herein comprising a porous hydrogel microsphere, results in a proportion of early apoptotic target cells that is higher than the proportion of early or late apoptotic target cells resulting from contacting the target cell with a comparator microsphere, wherein the comparator, microsphere comprises a smooth porous microsphere and a TNF-related apoptosis-inducing ligand (TRAIL), or active fragment thereof, wherein the TRAIL or active fragment thereof is attached to the surface of the smooth porous microsphere.

In aspects, the method of administering the synthetic cytotoxic cell or the composition disclosed herein comprising a porous hydrogel microsphere, results in a proportion of late apoptotic target cells that is higher than the proportion of early or late apoptotic target cells resulting from contacting the target cell with a soluble form of TRAIL or active fragment thereof.

In aspects, the method of administering the synthetic cytotoxic cell or the composition disclosed herein comprising a porous hydrogel microsphere, results in a proportion of late apoptotic target cells that is higher than the proportion of early or late apoptotic target cells resulting from contacting the target cell with a comparator microsphere, wherein the comparator microsphere comprises a non-hydrogel microsphere and a TNF-related apoptosis-inducing ligand (TRAIL), or active fragment thereof, wherein the TRAIL or active fragment thereof is attached to the surface of the non-hydrogel microsphere.

In aspects, the method of administering the synthetic cytotoxic cell or the composition disclosed herein comprising a porous hydrogel microsphere, results in a proportion of late apoptotic target cells that is higher than the proportion of early or late apoptotic target cells resulting from contacting the target cell with a comparator microsphere, wherein the comparator microsphere comprises a microsphere and a TNF-related apoptosis-inducing ligand (TRAIL), or active fragment thereof, wherein the TRAIL or active fragment thereof is encapsulated by the microsphere, optionally, wherein the microsphere is comprised of poly (lactic-co-glycolic acid).

In aspects, the method of administering the synthetic cytotoxic cell or the composition disclosed herein comprising a porous hydrogel microsphere, results in a proportion of late apoptotic target cells that is higher than the proportion of early or late apoptotic target cells resulting from contacting the target cell with a comparator microsphere, wherein the comparator microsphere comprises a smooth porous microsphere and a TNF-related apoptosis-inducing ligand (TRAIL), or active fragment thereof, wherein the TRAIL or active fragment thereof is attached to the surface of the smooth porous microsphere.

Administration of the synthetic cytotoxic cells, the pharmaceutical compositions, or components of the kits disclosed herein can occur by infusion (e.g., continuous or bolus), injection, consumption, electro-osmosis, hemodialysis, iontophoresis, and other methods known in the art. The mode of administration is not limited and may be intraarterial, intracranial, intradermal, intraduodenal, intramammary, intrameningeal, intraperitoneal, intrathecal, intratumoral, intravenous, intravitreal, ophthalmic, parenteral, spinal, subcutaneous, ureteral, urethral, vaginal, or intrauterine. In aspects, administration route is local or systemic.

In aspects, the synthetic cytotoxic cells, the pharmaceutical compositions, or components of the kits disclosed herein are administered in combination with additional therapeutic composition(s). In aspects, the synthetic cytotoxic cells, the pharmaceutical compositions, or components of the kits disclosed herein, and the additional therapeutic composition(s) are administered simultaneously. In aspects, the synthetic cytotoxic cells, the pharmaceutical compositions, or components of the kits disclosed herein is administered before the additional therapeutic composition(s). In aspects, the synthetic cytotoxic cells, the pharmaceutical compositions, or components of the kits disclosed herein is administered after the additional therapeutic composition(s).

In aspects, administration of the synthetic cytotoxic cells, the pharmaceutical compositions, or components of the kits disclosed herein disclosed herein in combination with the additional therapeutic composition(s) results in an enhanced therapeutic effect (e.g. when targeting a disease, such as cancer) than is observed by treatment with either the synthetic cytotoxic cells, the pharmaceutical compositions, or components of the kits disclosed herein or the additional therapeutic composition alone. In aspects, the cancer is resistant, refractory, or insensitive to treatment by the additional therapeutic composition alone. In aspects, the cancer is partially resistant, partially refractory, or partially insensitive to treatment by the additional therapeutic composition alone.

In aspects, the additional therapeutic composition comprises an immune checkpoint inhibitor. Several immune checkpoint inhibitors are known in the art and have received FDA approval for the treatment of one or more cancers. For example, FDA-approved PD-L1 inhibitors include Atezolizumab (Tecentriq®, Genentech), Avelumab (Bavencio®, Pfizer), and Durvalumab (Imfinzi®, AstraZeneca); FDA-approved PD-1 inhibitors include Pembrolizumab (Keytruda®, Merck) and Nivolumab (Opdivo®, Bristol-Myers Squibb); and FDA-approved CTLA4 inhibitors include Ipilimumab (Yervoy®, Bristol-Myers Squibb). Additional inhibitory immune checkpoint molecules that may be the target of future therapeutics include A2AR, B7-H3, B7-H4, BTLA, IDO, LAG3 (e.g., BMS-986016, under development by BSM), KIR (e.g., Lirilumab, under development by BSM), TIM3, TIGIT, and VISTA.

In aspects, the additional therapeutic composition comprises CAR expressing immune effector cells. Non-limiting examples of such CARS include CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J Natl Cancer Inst (2014) 107(1):364), carbonic anhydrase K-specific CARs (Lamers et al., Biochem Soc Trans (2016) 44(3):951-959), FR-α-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL13Ru2-specific CARs (Brown et al., Clin Cacner Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2):154-166), MSLN-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (Yescarta*) and Tisagenlecleucel (Kymriah®). See also, Li et al., J Hematol and Oncol (2018) 11(22), reviewing clinical trials of tumor-specific CARs.

In aspects, the additional therapeutic composition comprises a TRAIL- or TRAIL receptor-based cancer therapy. For instance, in aspects, the second therapeutic composition is Dulanermin, SCB-313, ABBV-621, Mapatumumab, Tigatuzumab, INBRX-109, IGM-8444, GEN1029, or BI 905711. Other TRAIL or TRAIL-receptor-based cancer therapies, which can be used as the additional therapeutic composition comprises, are listed in de Miguel D, et al. Onto better TRAILs for cancer treatment. Cell Death Differ. 2016 May; 23(5):733-47, which is herein incorporated by reference in its entirety.

### EXAMPLES

The present invention is further illustrated by reference to the following Examples. However, it should be noted that these Examples, like the embodiments described above, are illustrative and are not to be construed as restricting the scope of the invention in any way.

### Example 1: Generation of Porous Hydrogel Microspheres

Porous hydrogel microspheres were prepared with 9 weight percent (wt. %) PEG. In the aqueous phase, both acrylamide and bis-acrylamide were used at 0.62M to create the polymer hydrogel, with the addition of 0.0036M of streptavidin-acrylamide dissolved in a 100 mM pH 7.5 Tris-HCl buffer. Polyethylene glycol (PEG) is an inert, pore-forming agent. Linear PEG 8000 was used as an additive in the aqueous phase at 9 wt. % which creates hydrophobic pockets and consequently, leads to a displacement of volume in the hydrogel scaffold and thereby, the formation of macropores.

In the microfluidic PDMS device, the channels, flow rates and pressures were designed to allow for a ˜-20 μm raw droplet to be formed. Once the droplets were collected, they were de-gassed with argon to displace ambient oxygen molecules. Once the hydrogel scaffold was thermally cured in the presence of the polymerization agent known as ammonium persulfate at 0.1 wt. %, the hydrogel matrix emulsion was stable and could maintain their mechanical structure. The hydrogel matrix was then broken up by a 1:1 ratio of 1H, 1H, 2H, 2H-Perfluorooctan-1-ol (PFO). Subsequently, the hydrogel microspheres were washed and purified several times with water. This process produced a phase separation which liberated the coiled, collapsed PEG chains to be discarded away.

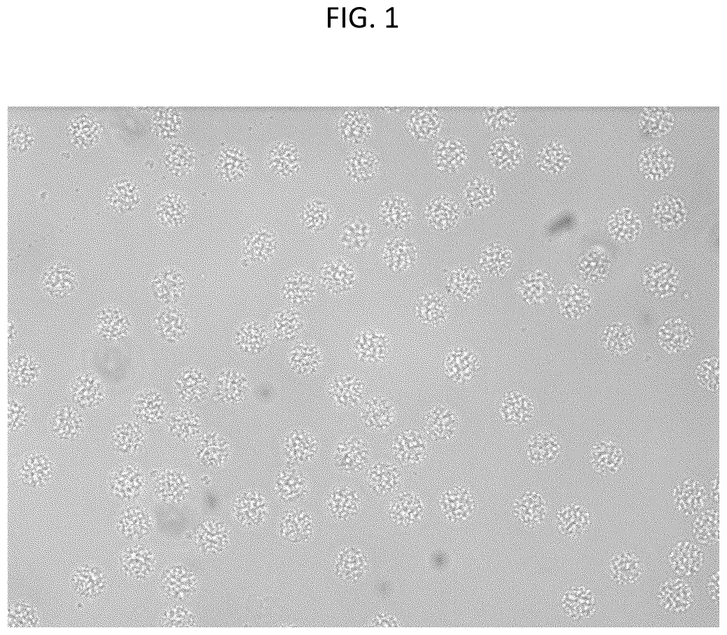

A confocal image of the purified porous hydrogel microspheres prepared with 9 wt. % PEG and comprising both micropores and macropores is shown in FIG. 1. Unlike microspheres generated using a crude bulk method such as vortex/homogenization, the porous hydrogel microspheres disclosed herein are very similar in size (i.e., the polydispersity index of the microspheres is very small).

### Example 2: Generation of Poly-Lactic-Co-Glycolic Acid (PLGA) Microspheres

Microspheres based on poly-lactic-co-glycolic-acid-carboxy (PLGA) were prepared as described below. This example utilized an 85:15 ratio of lactide to glycolide with a functional carboxy acid termination. Briefly, 3 w/v % PLGA (carboxy acid terminate) was used as the oil phase, and 1% polyvinyl alcohol (PVA) dissolved in distilled water was used as the water phase. A microfluidic PDMS device was used to combine the two phases into a water-in-oil emulsion with appropriate pressures and flow rates, allowing for a ˜10 μm droplet to be formed. Droplets were collected in a glass beaker on a magnetic hot plate containing a magnetic stir bar (100 rpm) and 50 mL of 1% PVA. The beaker was left overnight to evaporate the DCM and let the microspheres harden. After hardening, the microspheres were purified with washing with PBS or distilled water several times.

The microsphere's surface was modified by 1-Ethyl-3-(3-dimethylaminopropyl)carbodiimide (EDC) and Sulfo-NHS on the carboxyl acid terminated end of the polymer in order to attach streptavidin.

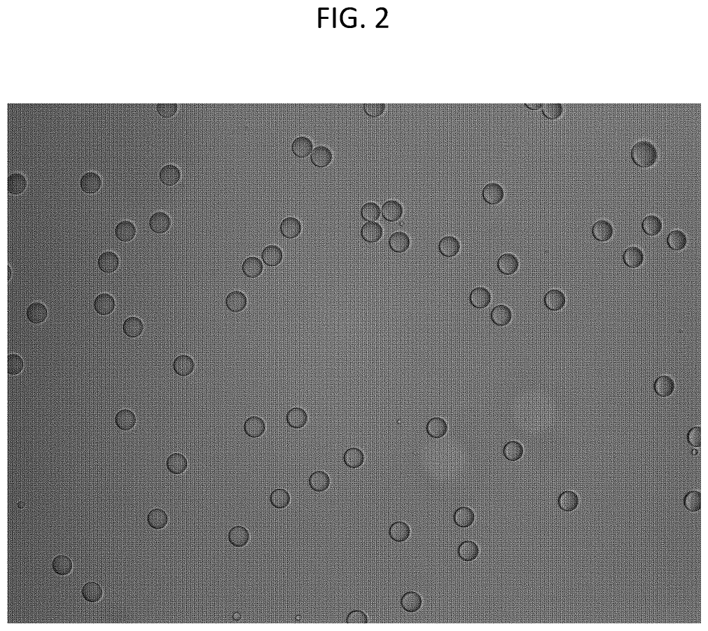

A confocal image of the purified 85:15 PLGA microspheres is shown in FIG. 2. Unlike microspheres generated using a crude bulk method such as vortex/homogenization, the PLGA microspheres disclosed herein are very similar in size (i.e., the polydispersity index of the microspheres is very small).

### Example 3: Generation of Synthetic Cytotoxic Cells

To generate synthetic cytotoxic cells, biotinylated TRAIL protein fragments comprising an amino acid sequence of SEQ ID NO: 2 were attached to the surface of the microspheres (both the porous hydrogels and the 85:15 PLGA) via biotin-streptavidin bond.

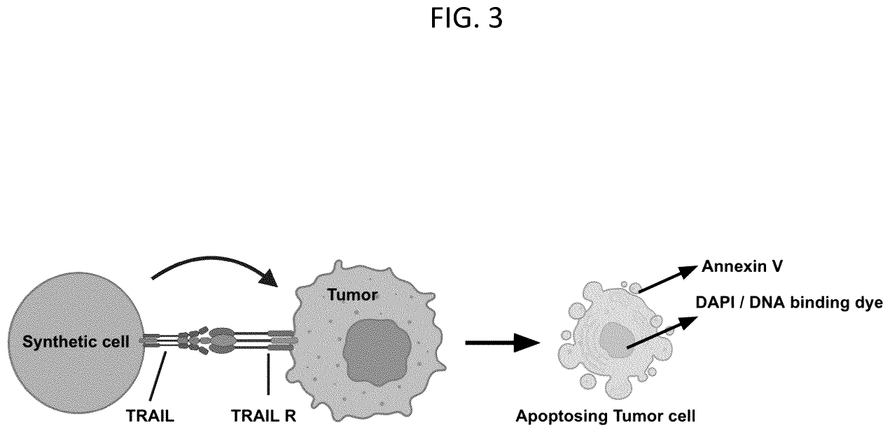

As illustrated in FIG. 3, the TRAIL protein fragment on the surface of synthetic cytotoxic cells can engage with TRAIL death receptors DR4 and DR5 expressed on cancer cells, causing apoptosis and/or necrosis of cancer cells. The apoptosis can be quantified by Annexin V and DAPI/DNA binding dye.

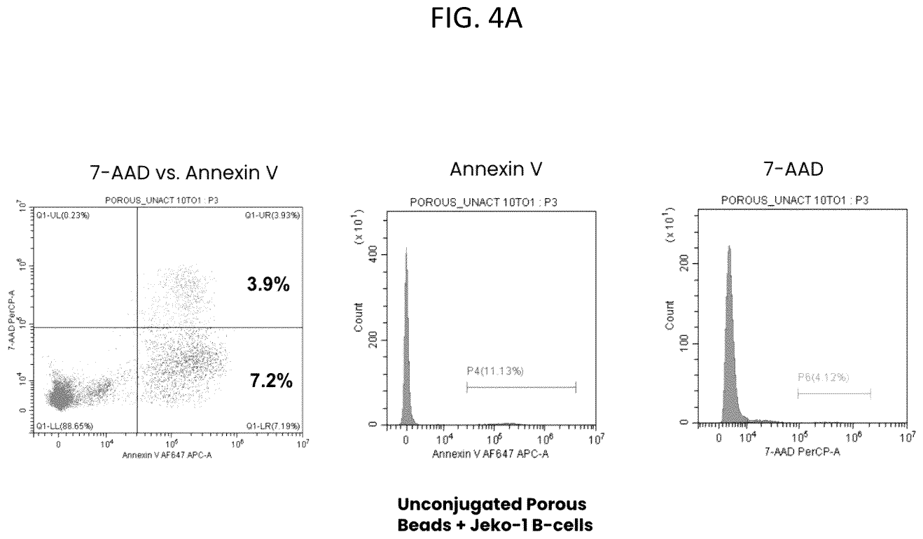

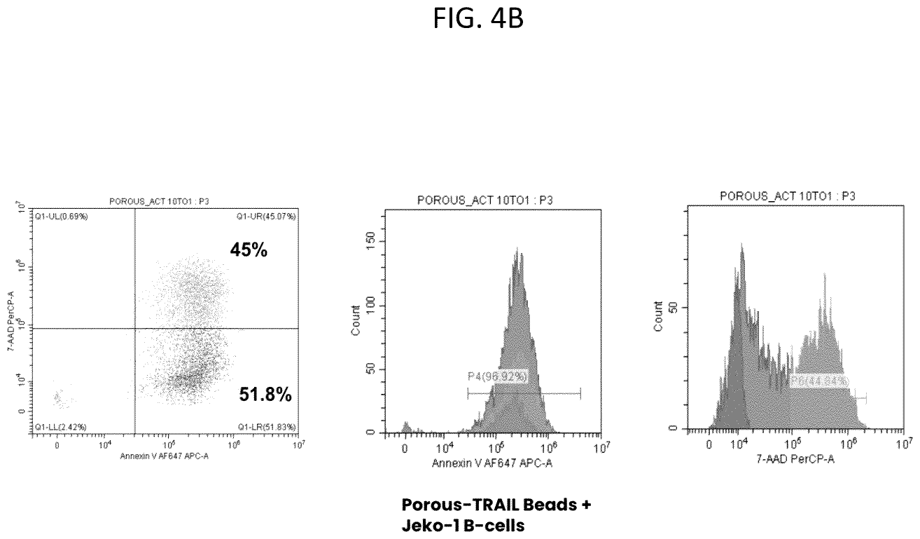

As shown in FIG. 4B, the synthetic cytotoxic cell based on porous hydrogels induced 96% of Jeko-1 MCL undergoing apoptosis and/or necrosis within 24 hours of co-culture. The killing efficiency was significantly higher than that of the control group shown in FIG. 4A. Without being bound by theory, it is thought that the compositions described herein provide a longer duration of action and increased efficacy than current strategies, such as administration of soluble or encapsulated TRAIL protein. Similar trends were observed 48 hours post co-culture (data not shown).

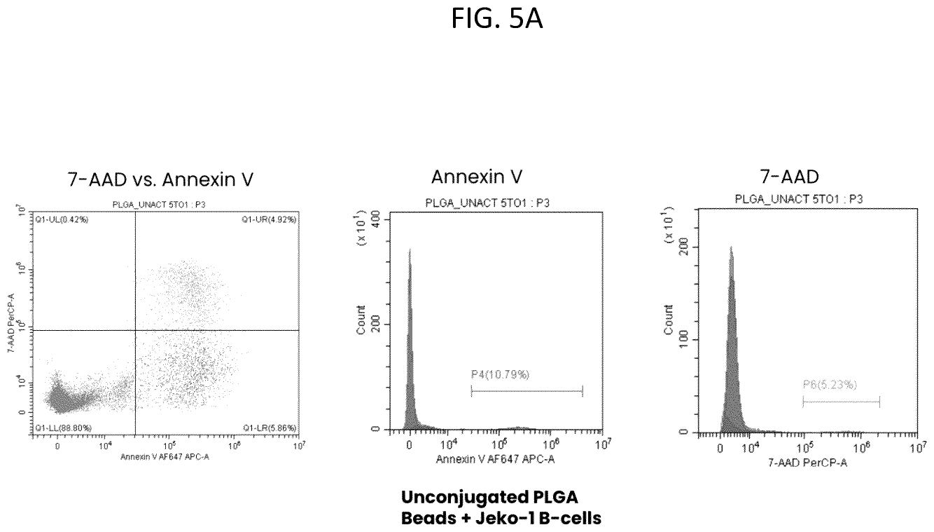

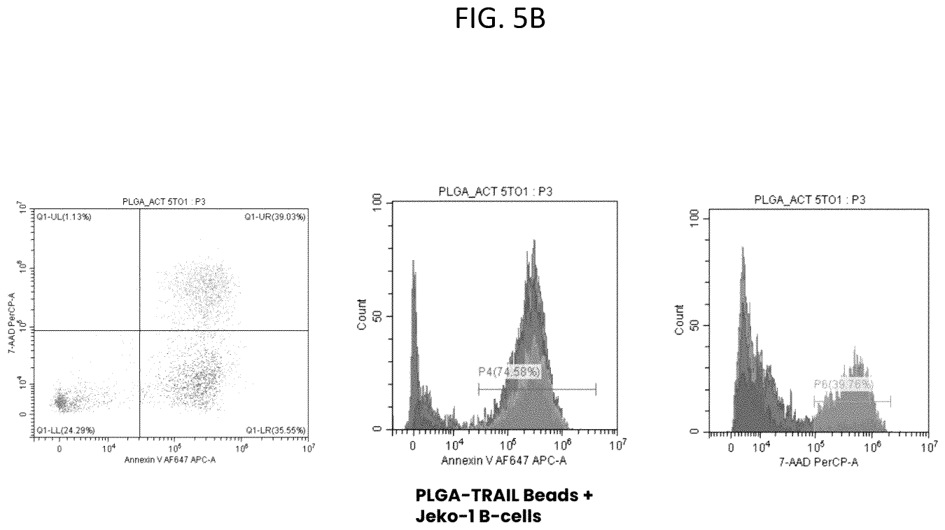

As shown in FIG. 5B, the synthetic cytotoxic cell based on 85:15 PLGA induced 76% of Jeko-1 MCL undergoing apoptosis and/or necrosis within 24 hours of co-culture. The killing efficiency was significantly higher than that of the control group shown in FIG. 5A. Without being bound by theory, it is thought that the compositions described herein provide a longer duration of action and increased efficacy than current strategies, such as administration of soluble or encapsulated TRAIL protein. Similar trends were observed 48 hours post co-culture (data not shown).

### Example 4: Generation of Streptavidin (STV) Porous Hydrogel Microspheres and STV Smooth Hydrogel Microspheres

### Generation of STV Porous Hydrogel Microspheres

To achieve a 20 μm droplet diameter, a flow-focusing geometry was used for two oil channels to focus a central stream of aqueous monomer solution to break off droplets in a water-in-oil emulsion. A fluorocarbon-oil (Novec 7500 3M, Inc., Lot 10311902) was used as the outer, continuous phase liquid for droplet formation. To stabilize droplets before polymerization, a surfactant (Krytox 157 FSH, Dupont, Lot #K6538/0374) was added at 2.0% w/w to the oil phase. In the aqueous phase, both acrylamide solution (Sigma Aldrich, Product #A4058), and bis-acrylamide/acrylamide solution (Sigma Aldrich, Product #A9926) were used at 0.62M final concentration to create the polymer hydrogel, with the addition of a final concentration of 0.2 mg/mL of (Thermo Fisher) streptavidin-acrylamide (Thermo Fisher) dissolved in a 100 mM pH 7.5 Tris-HCl buffer (Quality Biological Cat #351-006-101) and 0.1×PBS (VWR Life Science, Lot 23C2156941).

A linear Polyethylene glycol (PEG) polymer with an average molecular weight of 8000 (Sigma Aldrich, Lot #SLCD6888) was used as an additive in the aqueous phase at 9% (wt./vol) which creates hydrophobic pockets and consequently, a displacement of volume in the hydrogel scaffold. Therefore, PEG was used in the aqueous dispersion phase for microfluidic droplet generation synthetic cell synthesis. Adding PEG during the preparation of raw droplet hydrogels, followed by removal after polymerization, allows cavities and tunnels to be irreversibly introduced into the hydrogel matrix.

To convert the monomers into a polyacrylamide hydrogel, a radical initiator called ammonium persulfate 0.2% (wt./vol) was used in the aqueous formulation. Droplets were then sparged with argon gas for 15 minutes to evaluate oxygen molecules, and then thermally cured for 15 min at 75° C. FIG. 6B shows a brightfield image of these STV porous hydrogel microspheres.

### Generation of STV Smooth Hydrogel Microspheres

STV smooth hydrogel microspheres were generated using the same method as described above except that the 9% (wt./vol) PEG porogen was not used in the aqueous formulation. FIG. 6C shows a brightfield image of these STV smooth hydrogel microspheres.

Additionally, FIG. 6A shows a brightfield image of Streptavidin ACRO beads (also referred to herein as “STV magnetic beads”) of 5.5 μm diameter (BioSystem Acro ActiveMax® Streptavidin pBeads, premium grade (for cells); Cat. MBS-C009), which are superparamagnetic beads of 5.5 μm with streptavidin on their surface.

### Example 5: Generation of Surface-Attached TRAIL on Microspheres and Beads

To attach biotinylated TRAIL molecules to the surface of: (i) STV porous hydrogel microspheres, (ii) STV smooth hydrogel microspheres and (iii) STV magnetic beads, the following experiments were conducted.

### Quantitation of Available Streptavidin Molecules

A high-level overview of the available streptavidin molecules per sample type is summarized in Table 1. To quantify the available streptavidin molecules, each sample type ((i) STV porous hydrogel microspheres, (ii) STV smooth hydrogel microspheres, and (iii) STV magnetic beads) was stained using a PE-anti-streptavidin antibody (BioLegend, clone: 3A20.2). In parallel, one tube of BD Quantibrite™ (PE) Phycoerythrin Fluorescence Quantitation Kit (340495) was reconstituted and acquired in the same settings as samples as per manufacturer's instructions in the flow cytometer and the data obtained was used to plot a standard curve which was used to determine the number of streptavidin molecules on each of the different microspheres.

TABLE 1

| | STV porous | STV smooth | STV |
|---|---|---|---|
| | hydrogel | hydrogel | magnetic |
| Sample Type | microspheres | microspheres | beads |
| Sample | Porous bead | “Smooth | paramagnetic |
| Description | containing | hydrogels” | iron oxide |
| | macropores | containing | core; silica- |
| | generated | micropores | coated |
| | with 9% PEG | intrinsically | (ACRO bead ®) |
| | (Porous | (Smooth | |
| | microsphere) | microsphere) | |
| Avg. Diameter | 20 | 20 | 5.5 |
| (μm) | | | |
| Gel Fraction | 0.62M | 0.62M | N/A |
| (Molarity) | | | |
| # of STV | ~20,095 | ~20,095 | 120k |
| molecules/bead | | | |
| Bead generation | Droplet | Droplet | Unknown |
| method | microfluidics | microfluidics | (commercially |
| | | | available) |

### Biotinylation of Fc-TRAIL

An active fragment of the human TRAIL protein comprising the amino acid sequence of SEQ ID NO: 2 and having a mouse IgG2a-Fc Tag at the N-terminus (ACROBiosystems TRLH52591MG) was attached to an Oyo-link® single biotin from AlphaThera (AT-4001-1000). Biotinylation of Fc-TRAIL was performed using 1 mg of TRAIL-Fc Protein and 1 mg of Oyo-link® single biotin which resulted in a molar ratio of TRAIL: Biotinylation Reagent (1:3). Reaction was performed for two hours inside a LED PX2 Photo-Crosslinking Device (AT8001-D). After the two hours, the reaction mixture was concentrated six times using a 30 kDa cut off Amicon® filter to get rid of excess biotinylation reagent and to exchange the reaction buffer to HEPES. After the final concentration, the protein was resuspended at desired concentration in HEPES buffer supplemented with 10 μM Zn2+ to promote trimerization of TRAIL, and thereafter, used in subsequent conjugation and quantitation reaction.

### Conjugation of Biotinylated Fc-TRAIL to Microspheres or Beads

Biotinylated TRAIL protein at desired concentration was added to the protein conjugation buffer and the solution was mixed with 30 million STV porous hydrogel microspheres, STV smooth hydrogel microspheres, or STV magnetic beads in three independent reaction vessels and the vessels were rotated gently for 30 minutes. After completion of the reaction, the reaction vessels were washed twice with the conjugation buffer and the conjugated microspheres were immediately used downstream for quantitation and cellular assays.

### Quantitation of Biotinylated Fc-TRAIL Conjugated to Microspheres or Beads

The surface-attached TRAIL on microspheres or beads was quantitated as described below. 1 million each of TRAIL-conjugated STV porous microspheres, TRAIL-conjugated STV smooth microspheres, and TRAIL-conjugated STV magnetic beads were incubated with 100 μL of flow staining buffer containing 5 μL of TRAIL antibody conjugated with PE (Clone-RIK-2, BioLegend #BL-308206) for 30 minutes at room temperature in the dark. After staining, samples were washed with 1 mL of flow staining buffer twice, and 20000 events were recorded in the Cytek Aurora (5-Laser system). The quantification is performed with BD Quantibrite™ (PE) Phycoerythrin Fluorescence Quantitation Kit (340495), same as above. The corresponding amounts of TRAIL antigens conjugated to each bead type is shown in Table 2.

TABLE 2

| | Count of Biotinylated Fc-TRAIL |
|---|---|
| Sample Type | (molecules/bead) |
| STV porous hydrogel | 150,000 |
| microspheres | |
| STV smooth hydrogel | 220,000 |
| microspheres | |
| STV magnetic beads | 180,000 |

The results described above demonstrate that biotinylated TRAIL molecules are successfully attached to surface of: (i) STV porous hydrogel microspheres, (ii) STV smooth hydrogel microspheres, and (iii) STV magnetic beads.

### Example 6: Generation and Analysis of PLGA Microspheres Encapsulating TRAIL

To generate PLGA microspheres encapsulating biotinylated-TRAIL protein, 50:50 10 k Molecular weight PLGA was dissolved in dichloromethane (DCM) at 1% (wt./vol) and dissolved with free biotinylated-TRAIL at equivalent molecules estimated by the TRAIL conjugation method performed on the porous microspheres. 10 mL of 1% (wt./vol) polyvinyl alcohol (PVA) was added to the mixture. The solution was transferred to an IKA mixer and vortexed for 5 minutes at room temperature. The resulting solution was poured into 50 mL of 1% (wt./vol) PVA and stirred overnight. The following day, the resulting PLGA microspheres encapsulating biotinylated-TRAIL were washed with 1×PBS and used in cell culture experiments.

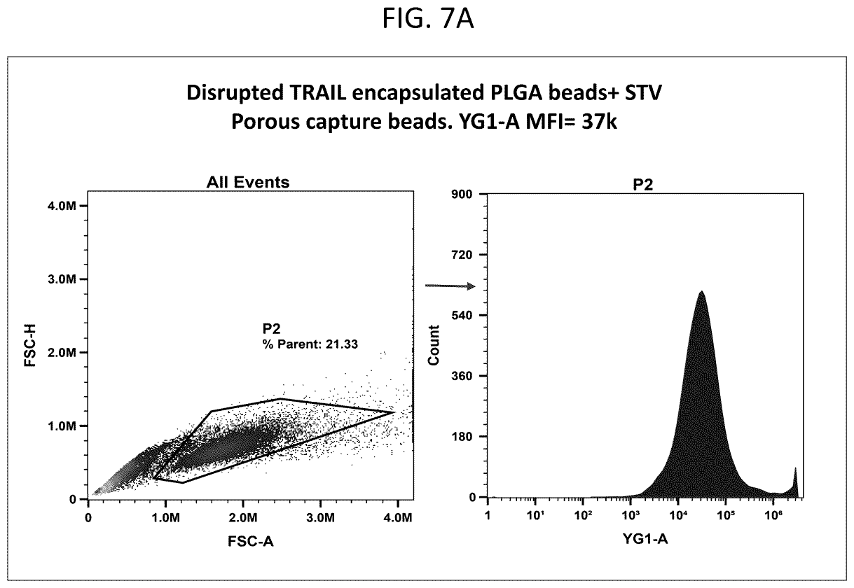

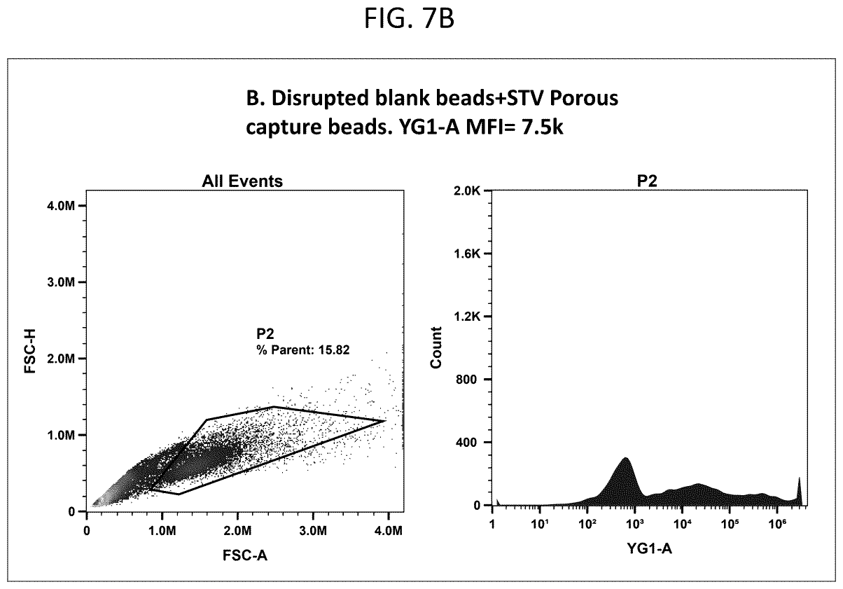

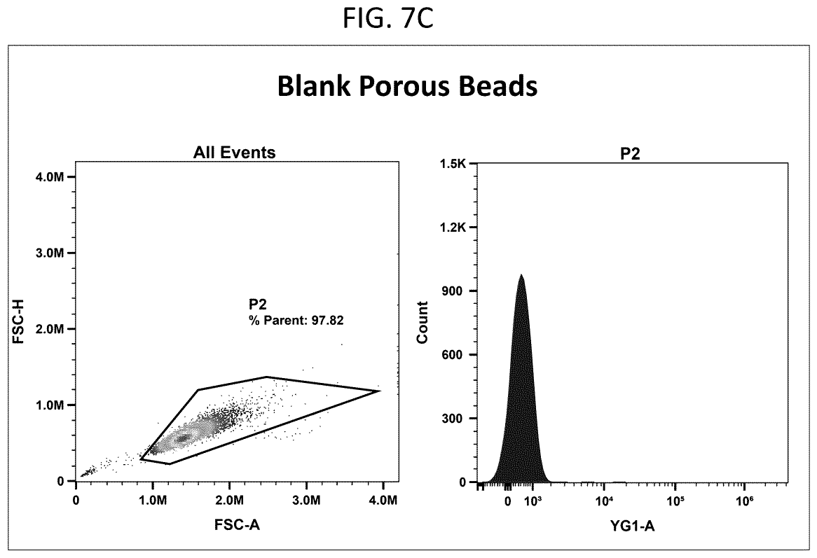

The amount of biotinylated TRAIL encapsulated was determined by microsphere disruption via sonication and incubation with STV porous hydrogel microspheres to capture the released biotinylated-TRAIL molecules. The STV porous hydrogel microspheres were then stained with anti-TRAIL antibody (PE) and quantified for TRAIL expression with flow cytometry. Quantitative fluorescent intensity flow plots reflecting the amount of TRAIL encapsulated in PLGA microspheres are shown in FIGS. 7A-C.

FIG. 7A is a flow plot showing the capture by STV porous hydrogel microspheres of biotinylated-TRAIL released from disrupted PLGA microspheres. The biotinylated TRAIL was labeled with anti-TRAIL antibody (APC). FIG. 7B shows a flow plot from a negative control experiment where STV porous hydrogel microspheres were used together with blank PLGA microspheres which do not contain any encapsulated TRAIL. FIG. 7C shows a flow plot of a negative control experiment comprising running blank porous beads on the same flow cytometer acquisition settings. This negative control is used to determine scatter match and for distinguishing porous beads shown in FIG. 7A.

These results demonstrate that biotinylated TRAIL molecules are successfully encapsulated in the PLGA microspheres using the procedure described above.

### Example 7: Hydrogel-Surface-Attached TRAIL is More Efficient at Killing Jeko-1 Tumor Cells in 24 Hours Relative to Comparator TRAIL Delivery Systems

Two tumor cell lines expressing TRAIL receptors DR4 and/or DR5, Jeko-1 (ATCC, CRL-3006™) and Jurkat (ATCC, E6-1 clone, TIB-152™), were chosen to demonstrate and evaluate the cytotoxic effects of the TRAIL delivery systems described above. Briefly, 100K live Jeko-1 or Jurkat cells were plated in RPMI media containing 10% Fetal bovine serum and 1% PenStrep in 100 μL volume.

To ensure equivalent TRAIL antigens were incubated with 100K Jeko-1 tumor cells or Jurkat tumor cells, the number of microspheres or beads was normalized to porous bead TRAIL antigen density as shown in Table 3, assuming 10 beads for every cell.

TABLE 3

| | # TRAIL | Normalized # of |
|---|---|---|
| Sample Type | antigens/bead | beads/well |
| STV porous hydrogel | 150,000 | 1e⁶ |
| microspheres | | |
| STV smooth hydrogel | 220,000 | 680k |
| microspheres | | |
| STV magnetic beads | 180,000 | 830k |
| PLGA Microspheres | 25e6 biotinylated TRAIL | 1e⁶ |
| Encapsulating TRAIL | molecules/bead (Formulation | |
| | made assuming 150k | |
| | antigens/bead) | |

Synthetic cytotoxic cells and equivalent total molecules of soluble TRAIL were plated 8 hours post seeding of tumor cells in 100 μL total volume. Amount of soluble TRAIL per well to match the amount of microsphere or bead-bound TRAIL was calculated as follows. Assuming 150K TRAIL molecules per microsphere/bead and 1e⁶ microsphere/bead per well, 1.5e¹¹ soluble TRAIL molecules per well were used, which was calculated to be equivalent to 23 ng soluble TRAIL per well.

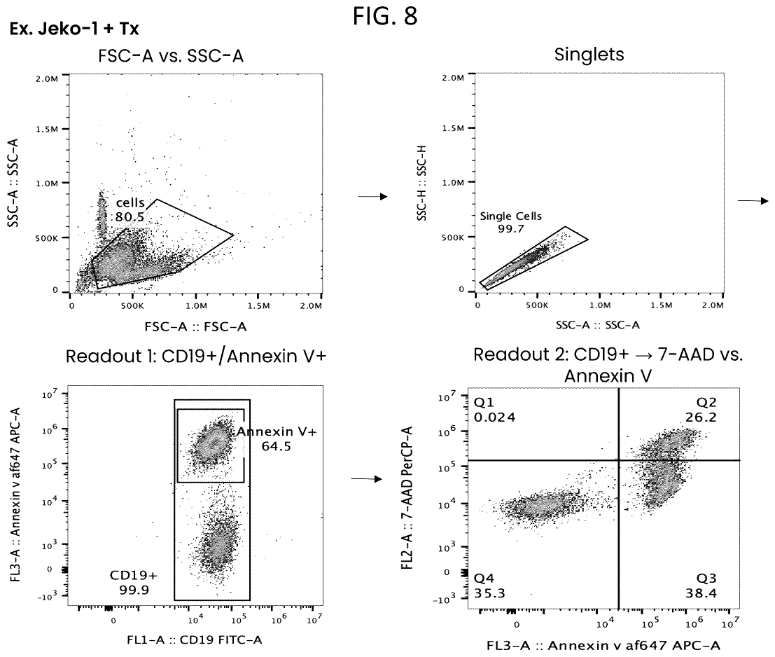

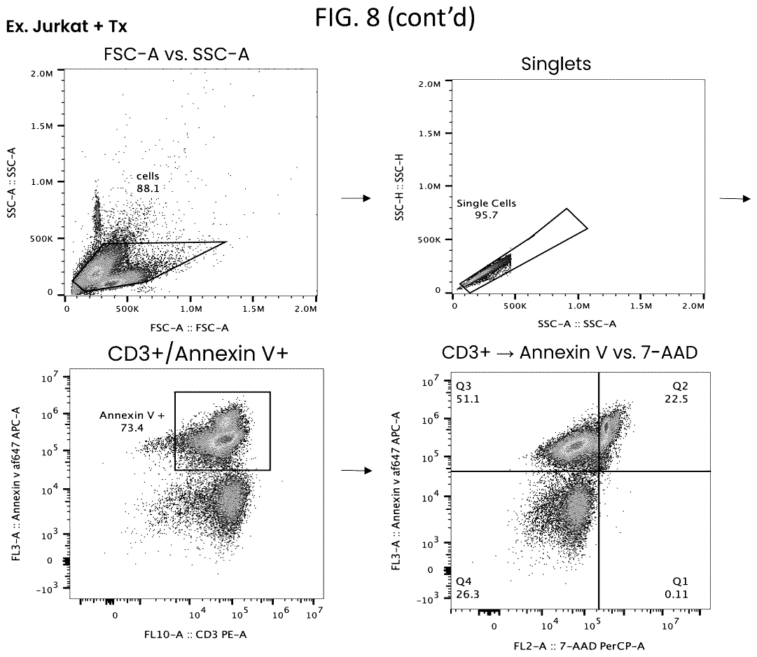

Twenty-four and forty-eight hours post incubation, cells were harvested and stained with Annexin V and 7-AAD. Anti-CD19 and Anti-CD3 antibodies were used to separate Jeko-1 cells from beads and Jurkat cells from beads, respectively. While Jeko-1 cells are CD19-expressing B-cells that are TRAIL-sensitive, Jurkat cells are CD3-expressing T cells that have low-moderate sensitivity to TRAIL. Determination of stage of cell death and % apoptotic cells were determined from the gating strategy outlined in FIG. 8. Samples were plated in duplicates and all sample acquisition was performed on the Cytoflex Beckman Coulter Flow Cytometer 24 hours and 48 hours post treatment of tumor cells with treatment conditions. Data analysis was further processed on FlowJo™ v10 software.

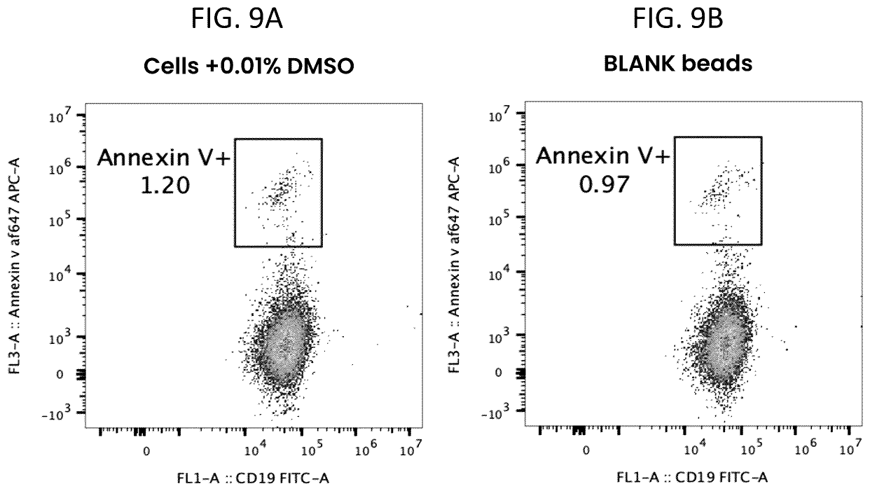

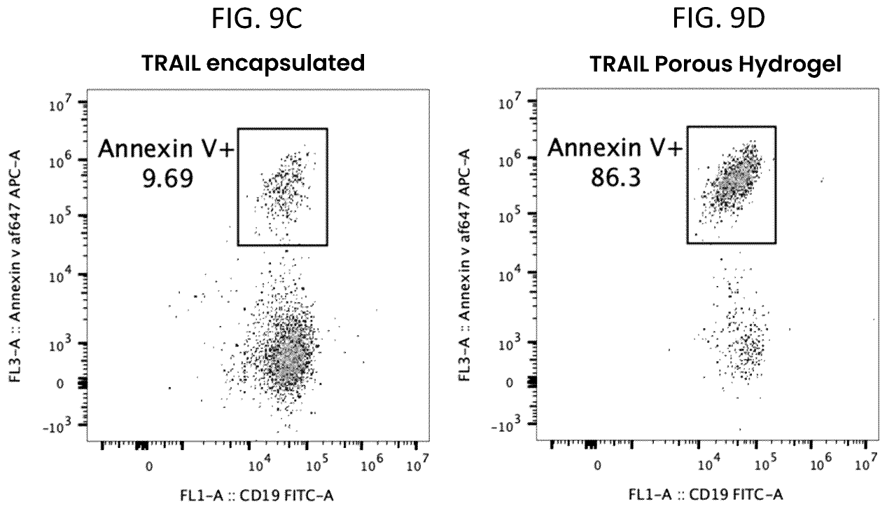

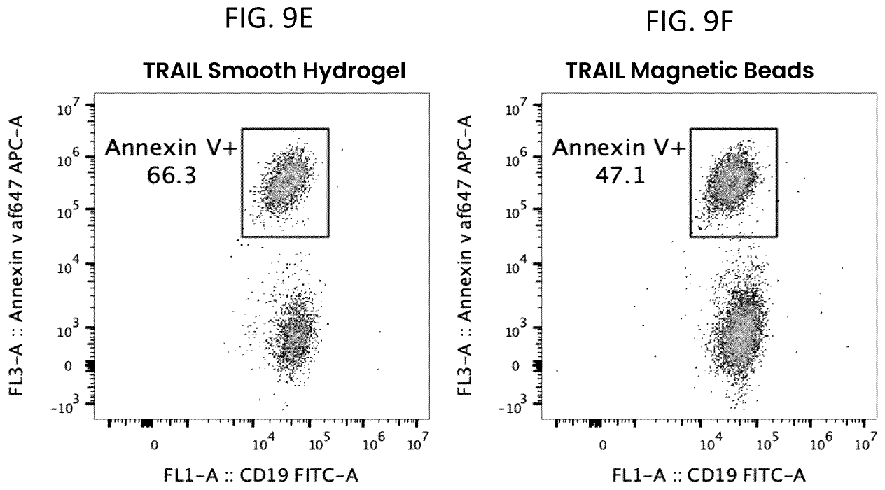

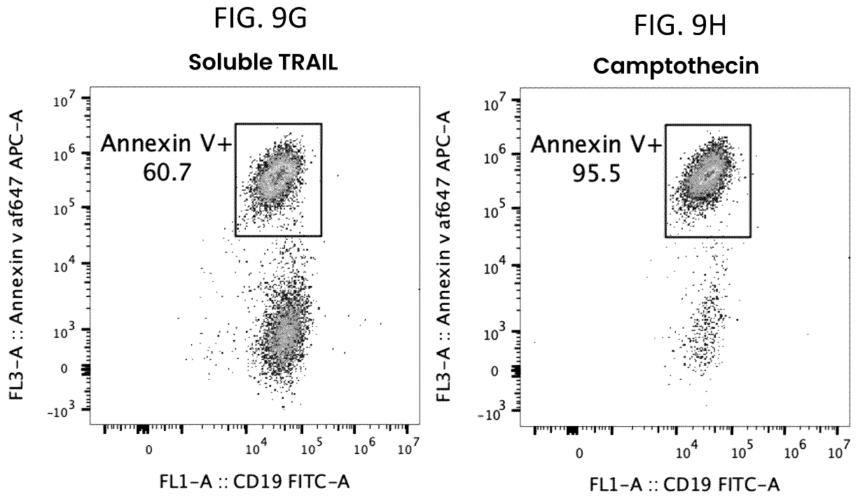

Treatment of Jeko-1 cells with TRAIL attached to the surface of porous hydrogel microspheres or smooth hydrogel microspheres results in a higher percentage of apoptotic cells (86.3% and 66.3% respectively; FIGS. 9D and 9E) compared to TRAIL encapsulated in PLGA microspheres (resulting in 9.69% apoptotic cells; FIG. 9C), TRAIL attached to magnetic beads (resulting in 47.10% apoptotic cells; FIG. 9F), or soluble TRAIL protein (60.7% apoptotic cells; FIG. 9G). See FIGS. 9A-9H, Table 4A.

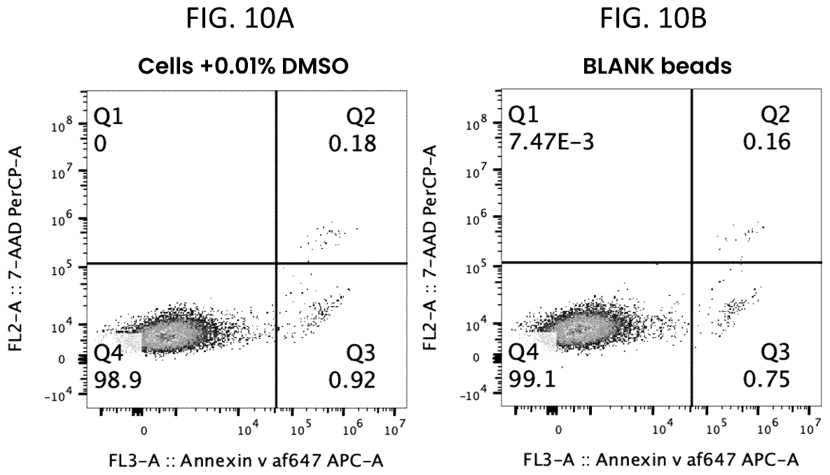

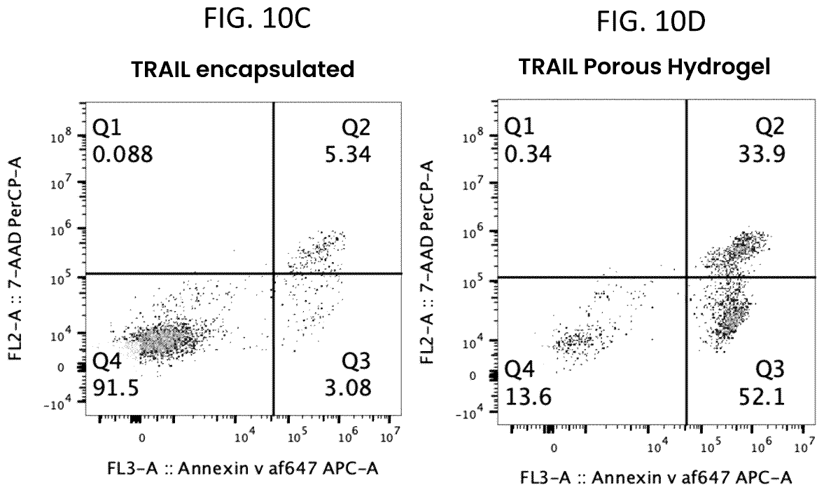

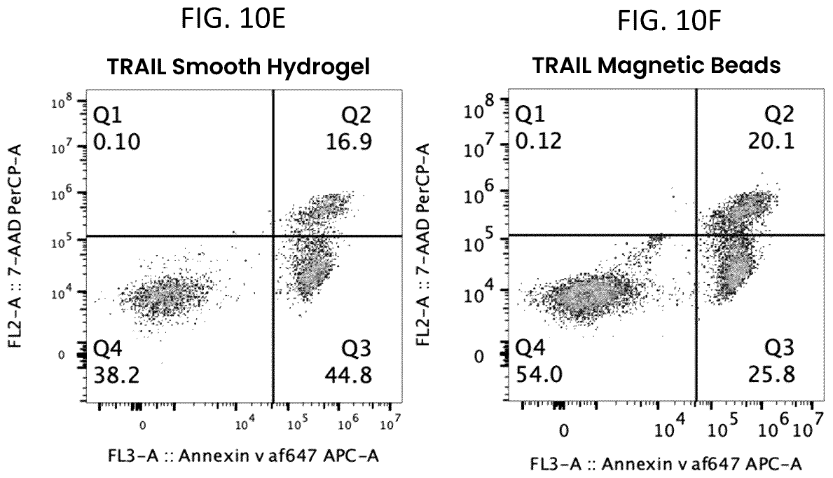

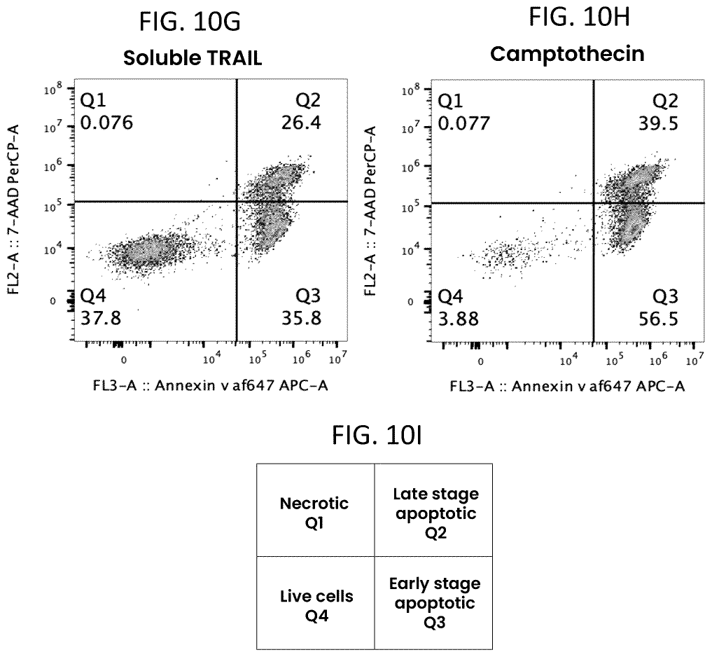

Moreover, treatment of Jeko-1 cells with TRAIL attached to the surface of porous hydrogel microspheres or smooth hydrogel microspheres results in a higher percentage of early apoptotic cells (52.3% and 44.8% respectively; Q3 of FIGS. 10D and 10E) as compared to TRAIL encapsulated in PLGA microspheres (resulting in 3.08% early apoptotic cells; FIG. 10C), TRAIL attached to magnetic beads (resulting in 25.8% early apoptotic cells; FIG. 10F), or soluble TRAIL protein (35.8% early apoptotic cells; FIG. 10G). See FIGS. 10A-10I.

These results demonstrate that attaching TRAIL to the surface of hydrogel microspheres as disclosed herein is a more efficient way to kill tumor cells as compared to encapsulating TRAIL in a microsphere or using soluble TRAIL. These results also highlight the role of the hydrogel microspheres disclosed herein, since attaching TRAIL to the surface of hydrogel microspheres was much more effective at killing tumor cells as compared to attaching TRAIL to the surface of magnetic (non-hydrogel) beads.

### Example 8: Surface-Attached TRAIL on Porous Hydrogel Microspheres is More Efficient at Killing Tumor Cells Relative to Comparator TRAIL Delivery Systems

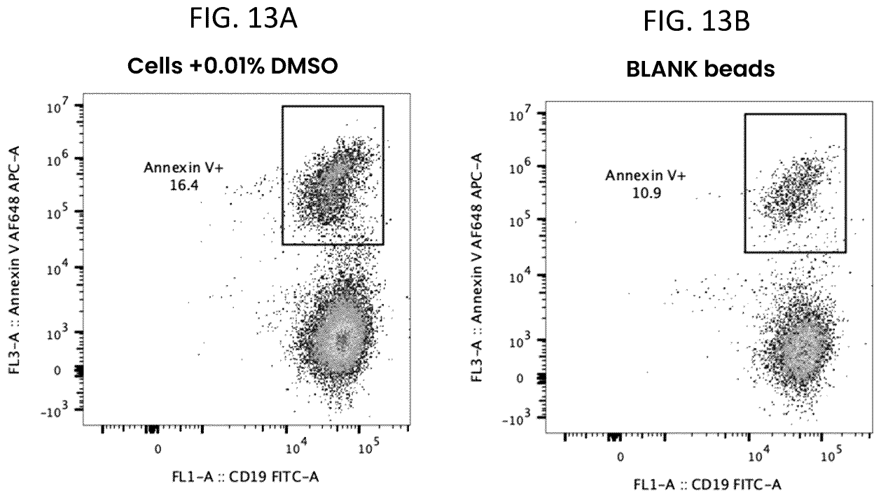

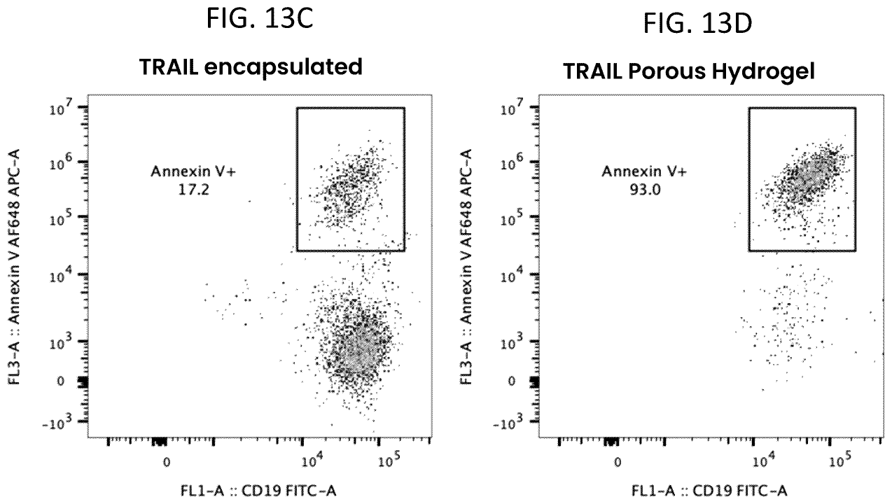

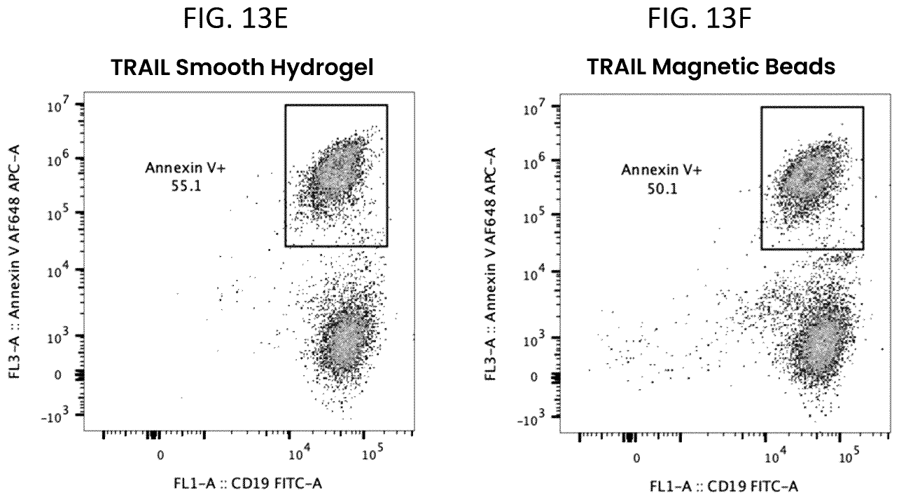

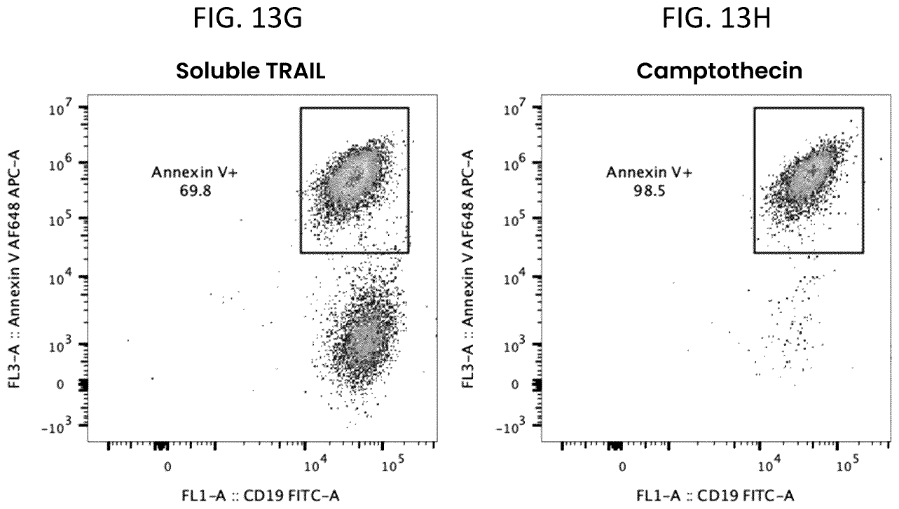

Treatment of Jeko-1 cells for 48 hours with TRAIL attached to the surface of porous hydrogel microspheres results in a remarkably higher percentage of apoptotic cells (93%; FIG. 13D) compared to TRAIL encapsulated in PLGA microspheres (resulting in 17.2% apoptotic cells; FIG. 13C), TRAIL attached to magnetic beads (resulting in 50.1% apoptotic cells; FIG. 13F), or soluble TRAIL protein (69.8% apoptotic cells; FIG. 13G). See FIGS. 13A-13H.

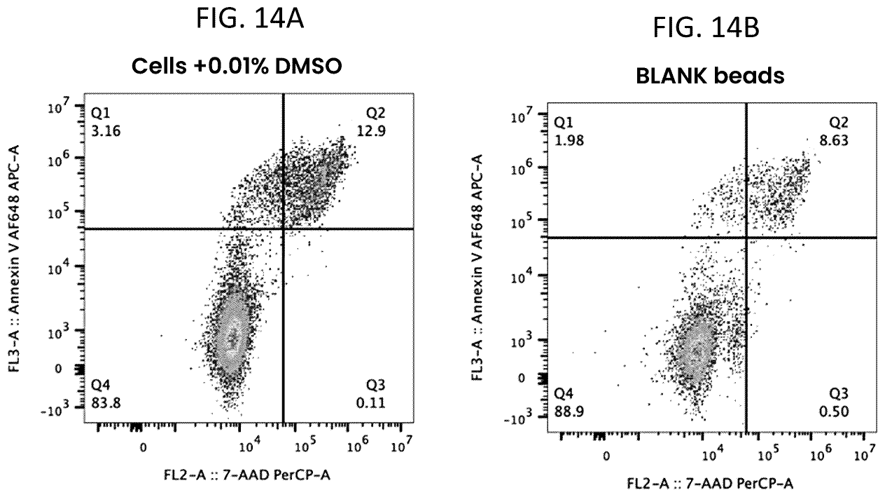

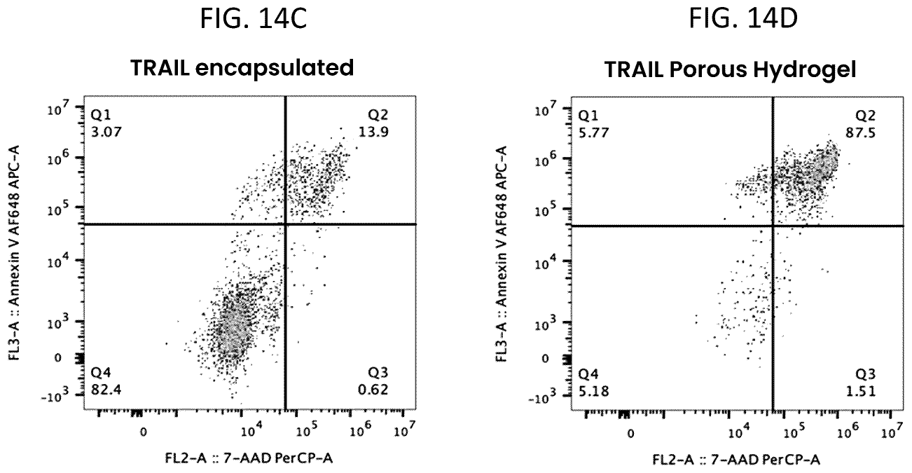

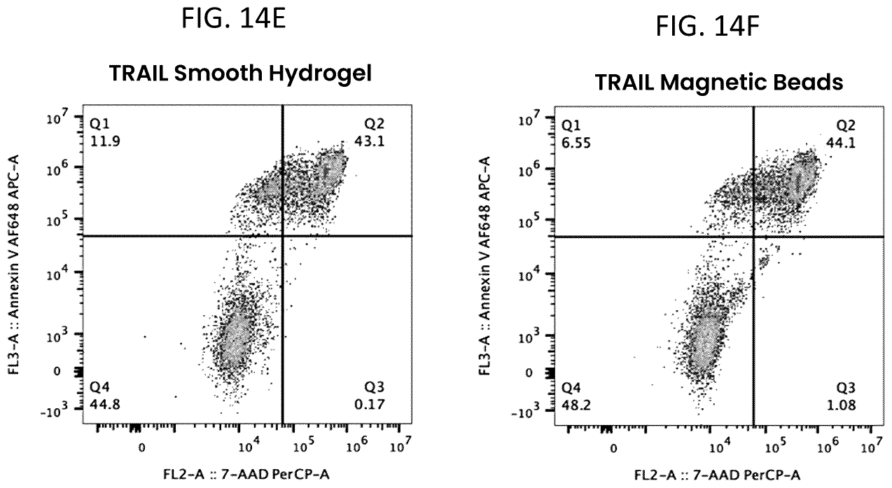

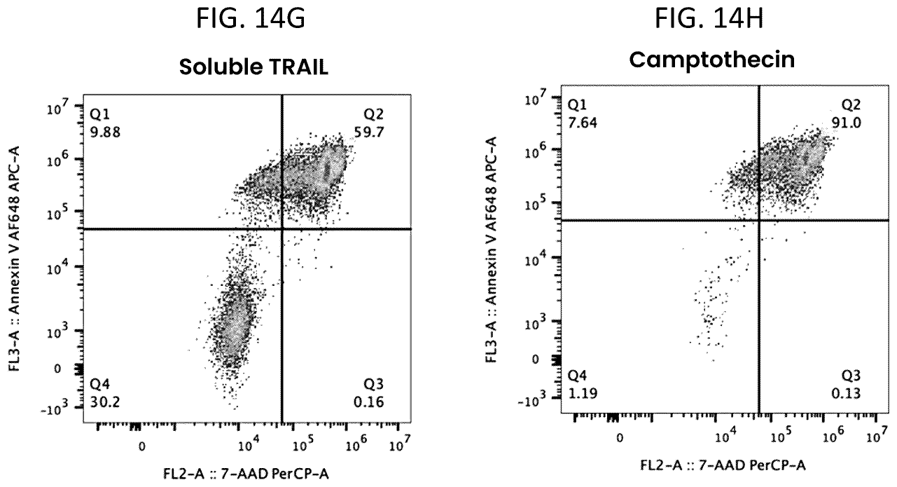

With regard to the stage of apoptosis, treatment of Jeko-1 cells for 48 hours with TRAIL attached to the surface of porous hydrogel microspheres results in a higher percentage of late apoptotic cells (87.5%; Q2 of FIG. 14D) as compared to TRAIL encapsulated in PLGA microspheres (resulting in 13.9% late apoptotic cells; FIG. 14C), TRAIL attached to magnetic beads (resulting in 44.1% late apoptotic cells; FIG. 14F), or soluble TRAIL protein (59.7% late apoptotic cells; FIG. 14G). See FIGS. 14A-14H.

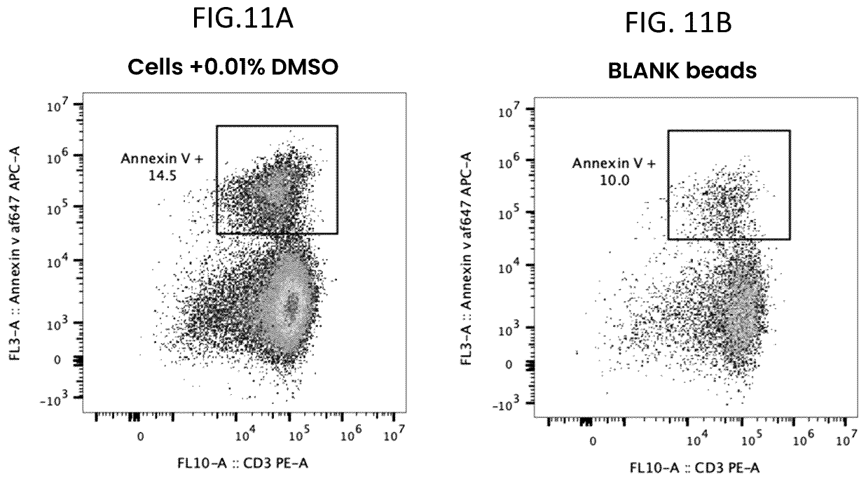

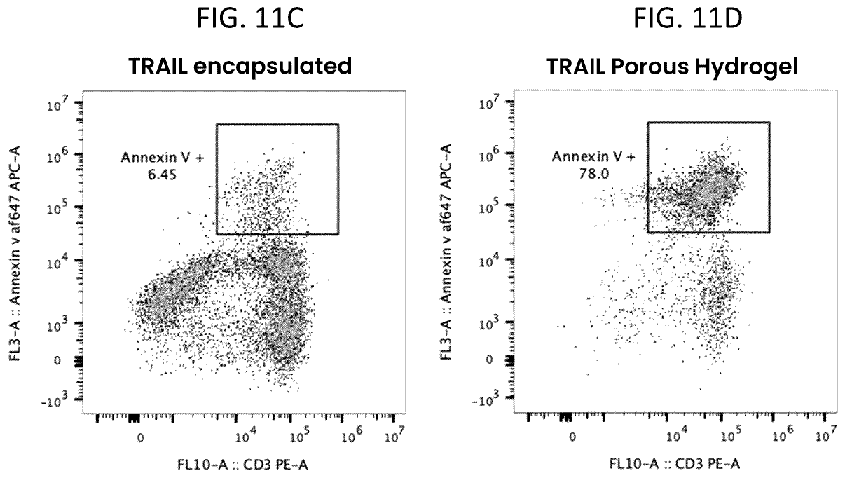

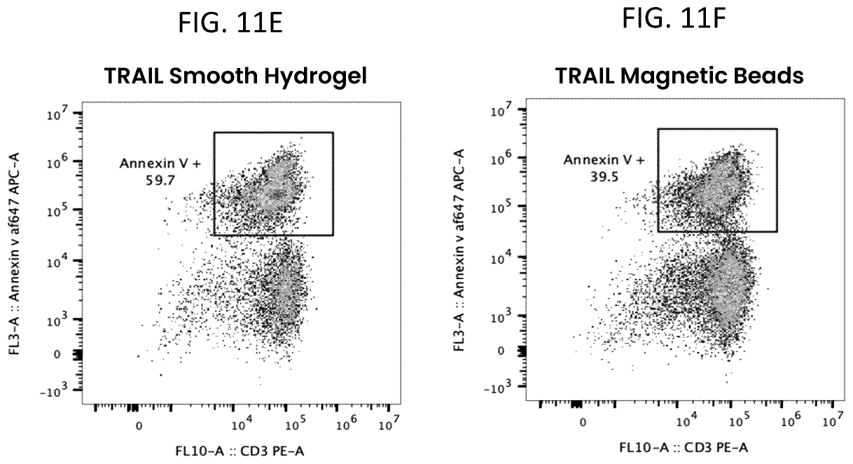

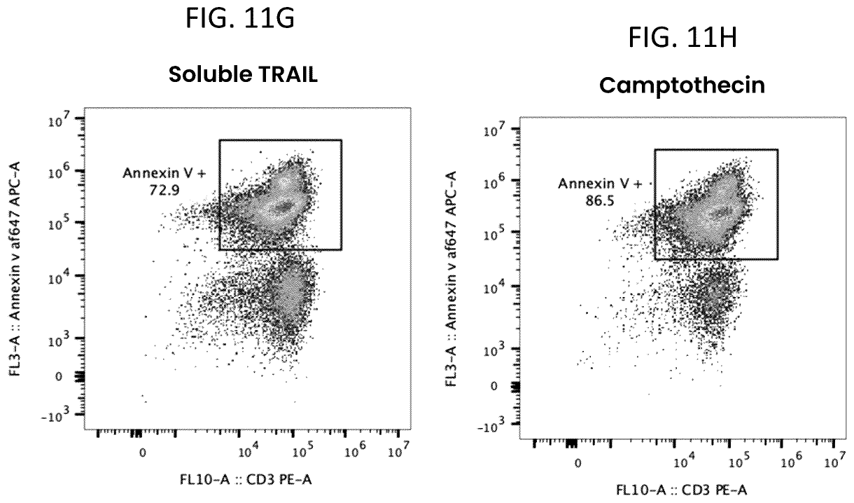

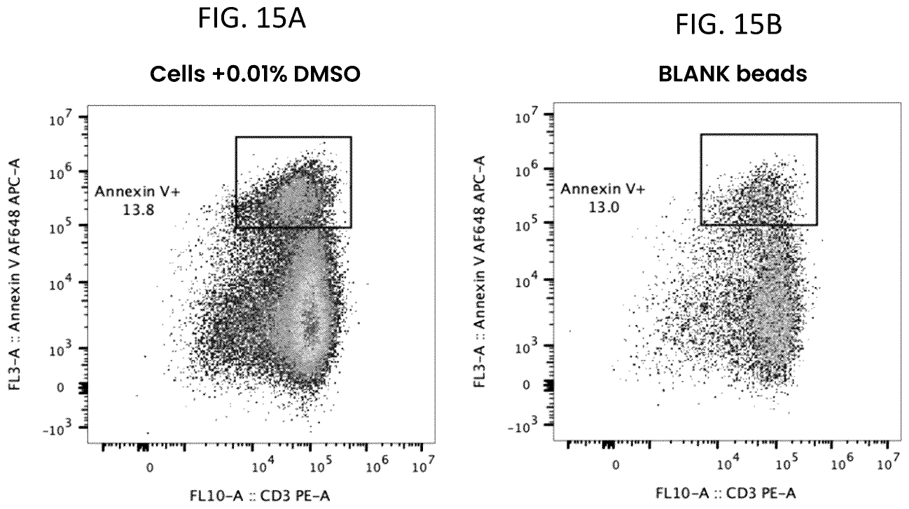

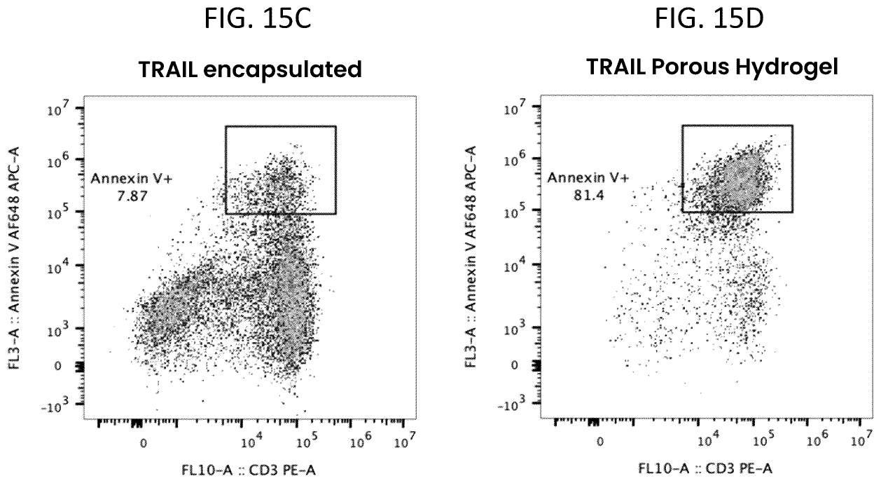

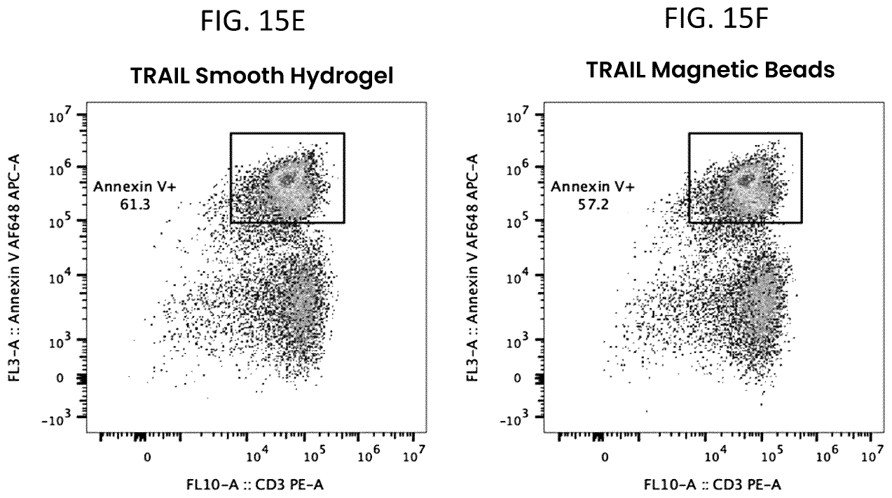

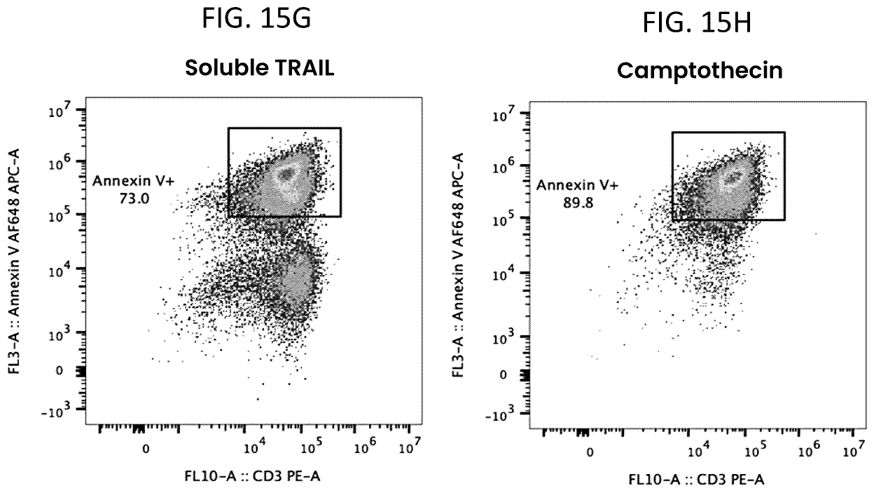

Treatment of Jurkat cells with TRAIL attached to the surface of porous hydrogel microspheres results in a remarkably higher percentage of apoptotic cells (78% at 24 h and 81.4% at 48 h; FIGS. 11D, 15D) compared to TRAIL encapsulated in PLGA microspheres (6.45% at 24 h and 7.87% at 48 h; FIGS. 11C, 15C), TRAIL attached to magnetic beads (39.5% at 24 h and 57.2% at 48 h; FIGS. 11F, 15F), or soluble TRAIL protein (72.9% at 24 h and 73% at 48 h; FIG. 11G). See FIGS. 11A-11H, FIGS. 15A-15H and Table 4B.

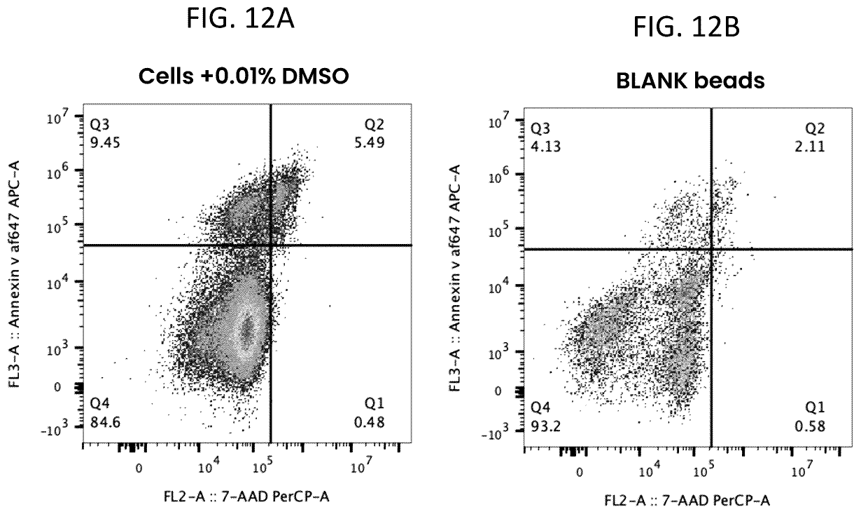

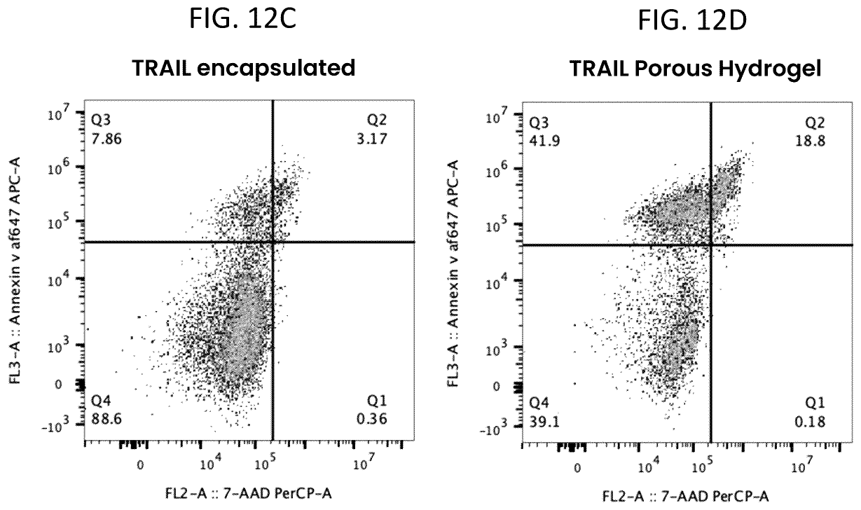

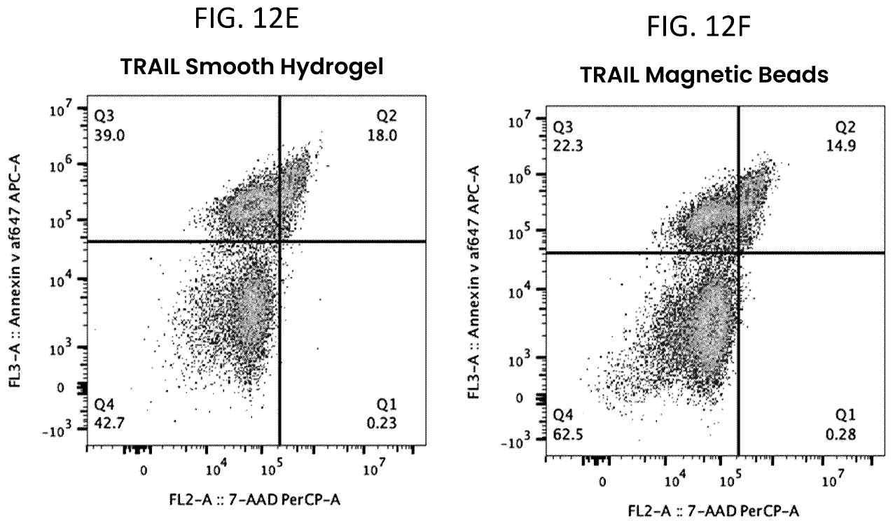

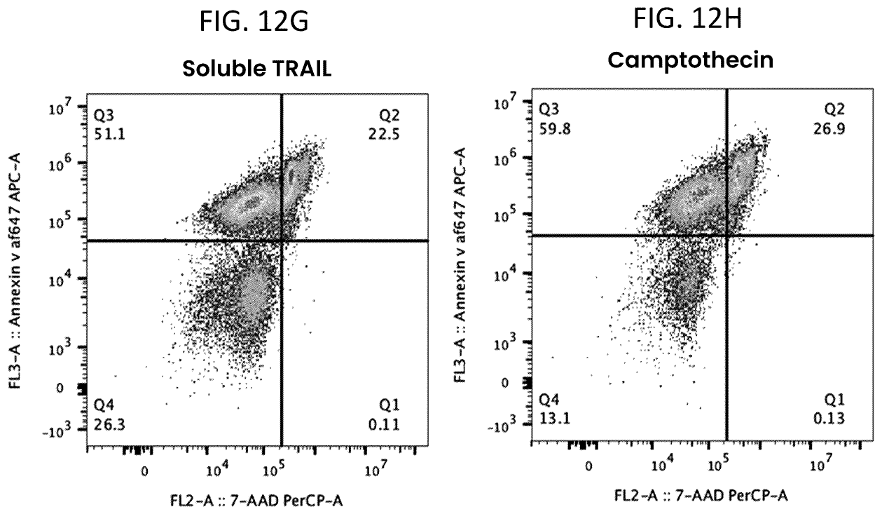

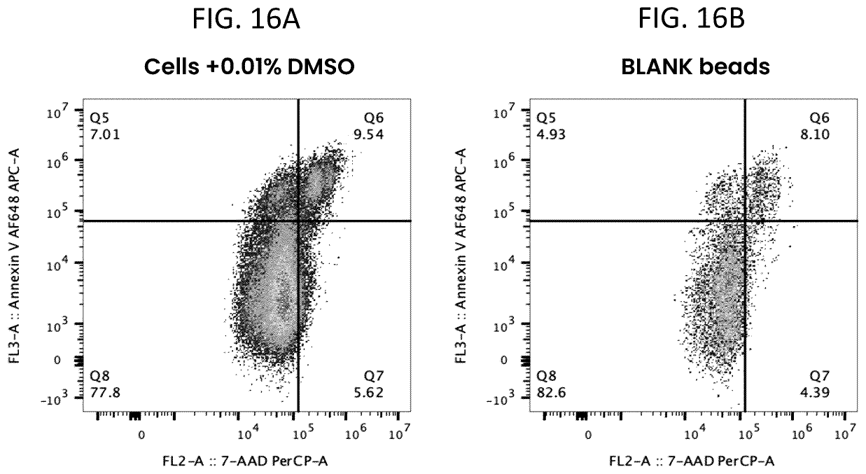

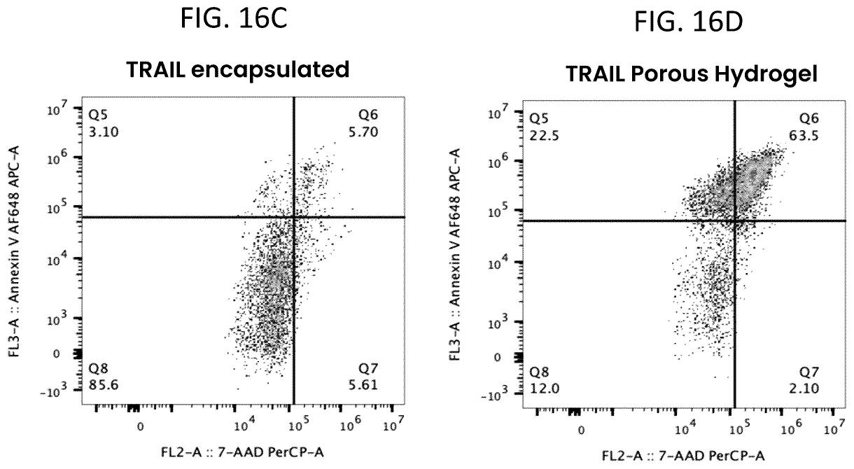

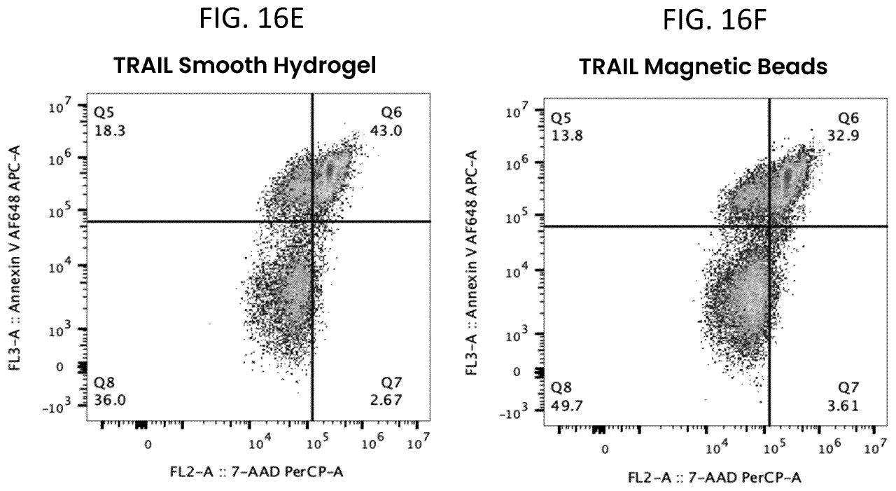

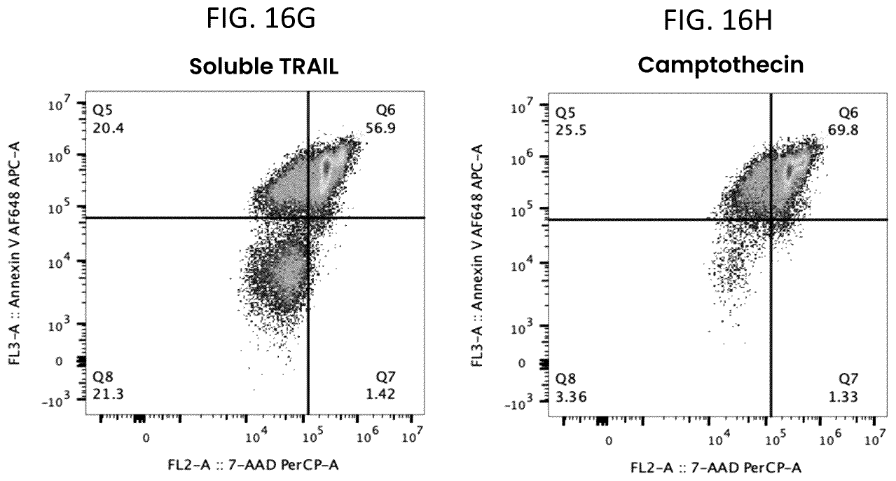

With regard to the stage of apoptosis, treatment of Jurkart cells for 48 hours with TRAIL attached to the surface of porous hydrogel microspheres results in a higher percentage of late apoptotic cells (63.5%; Q6 of FIG. 16D) as compared to TRAIL encapsulated in PLGA microspheres (5.7%; FIG. 16C), TRAIL attached to magnetic beads (32.9%; FIG. 16F), or soluble TRAIL protein (56.9%; FIG. 16G). See FIGS. 16A-16H. As a corollary, the percentage of live Jurkat cells is the least after 48 h treatment with TRAIL attached to the surface of porous hydrogel microspheres (12% live; Q8 of FIG. 16D), as compared to TRAIL encapsulated in PLGA microspheres (85.6% live; FIG. 16C), TRAIL attached to magnetic beads (49.7% live; FIG. 16F), or soluble TRAIL protein (21.3%; FIG. 16G). Also, Table 5A, 5B and FIGS. 12A-I.

The results described above show that surface-attached TRAIL on porous hydrogel microspheres is remarkably more effective in inducing apoptosis of tumor cells, compared to microspheres encapsulating TRAIL, soluble TRAIL, and surface-attached TRAIL on magnetic beads, thereby demonstrating the unexpectedly superior cytotoxic capabilities of the surface-attached TRAIL on porous hydrogel microspheres disclosed herein.

TABLE 4A

| | | | | TRAIL | TRAIL | | | |
|---|---|---|---|---|---|---|---|---|
| | Cells + | | TRAIL | Porous | Smooth | TRAIL | | |
| | 0.01% | Blank | encapsulated | Hydro | Hydro | Magnetic | Soluble | 100 nM |
| 24 Hrs. | DMSO | beads | PLGA | gels | gels | Beads | TRAIL | Camptothecin |
| Live Cells | 98.9 | 99.1 | 91.5 | 13.6 | 38.2 | 54.0 | 37.8 | 3.88 |
| (7-AAD−/ | | | | | | | | |
| Annexin | | | | | | | | |
| V−) | | | | | | | | |
| Early Stage | 0.92 | 0.75 | 3.08 | 52.1 | 44.8 | 25.8 | 35.8 | 56.5 |
| Apoptotic | | | | | | | | |
| (7-AAD−/ | | | | | | | | |
| Annexin | | | | | | | | |
| V+) | | | | | | | | |
| Late Stage | 0.18 | 0.16 | 5.34 | 33.9 | 16.9 | 20.1 | 26.4 | 39.5 |
| Apoptotic | | | | | | | | |
| (7-AAD+/ | | | | | | | | |
| Annexin | | | | | | | | |
| V+) | | | | | | | | |
| Necrotic | 0 | 7.47e−3 | 0.088 | 0.34 | 0.10 | 0.12 | 0.076 | 0.077 |
| (7-AAD+/ | | | | | | | | |
| Annexin | | | | | | | | |
| V−) | | | | | | | | |

TABLE 4B

| | | | | TRAIL | TRAIL | | | |
|---|---|---|---|---|---|---|---|---|
| | Cells + | | TRAIL | Porous | Smooth | TRAIL | | |
| | 0.01% | Blank | encapsulated | Hydro | Hydro | Magnetic | Soluble | 100 nM |
| 48 Hrs. | DMSO | beads | PLGA | gels | gels | Beads | TRAIL | Camptothecin |
| Live Cells | 83.8 | 88.9 | 82.4 | 5.18 | 44.8 | 48.2 | 30.2 | 1.19 |
| (7-AAD−/ | | | | | | | | |
| Annexin | | | | | | | | |
| V−) | | | | | | | | |
| Early Stage | 3.16 | 1.98 | 3.07 | 5.77 | 11.9 | 6.55 | 9.88 | 7.64 |
| Apoptotic | | | | | | | | |
| (7-AAD−/ | | | | | | | | |
| Annexin | | | | | | | | |
| V+) | | | | | | | | |
| Late Stage | 12.9 | 8.63 | 13.9 | 87.5 | 43.1 | 44.1 | 59.7 | 91.0 |
| Apoptotic | | | | | | | | |
| (7-AAD+/ | | | | | | | | |
| Annexin | | | | | | | | |
| V+) | | | | | | | | |
| Necrotic | 0.11 | 0.50 | 0.62 | 1.51 | 0.17 | 1.08 | 0.16 | 0.13 |
| (7-AAD+/ | | | | | | | | |
| Annexin | | | | | | | | |
| V−) | | | | | | | | |

TABLE 5A
Table 5A.

| | | | | TRAIL | TRAIL | | | |
|---|---|---|---|---|---|---|---|---|
| | Cells + | | TRAIL | Porous | Smooth | TRAIL | | |
| | 0.01% | Blank | encapsulated | Hydro | Hydro | Magnetic | Soluble | 100 nM |
| 24 Hrs. | DMSO | beads | PLGA | gels | gels | Beads | TRAIL | Camptothecin |
| Live Cells | 84.6 | 93.2 | 88.6 | 39.1 | 42.7 | 62.5 | 26.3 | 13.1 |
| (7-AAD−/ | | | | | | | | |
| Annexin | | | | | | | | |
| V−) | | | | | | | | |
| Early Stage | 9.45 | 4.13 | 7.86 | 41.9 | 39.0 | 22.3 | 51.1 | 59.8 |
| Apoptotic | | | | | | | | |
| (7-AAD−/ | | | | | | | | |
| Annexin | | | | | | | | |
| V+) | | | | | | | | |
| Late Stage | 5.49 | 2.11 | 3.17 | 18.8 | 18.0 | 14.9 | 22.5 | 26.9 |
| Apoptotic | | | | | | | | |
| (7-AAD+/ | | | | | | | | |
| Annexin | | | | | | | | |
| V+) | | | | | | | | |
| Necrotic | 0.48 | 0.58 | 0.36 | 0.18 | 0.23 | 0.28 | 0.11 | 0.13 |
| (7-AAD+/ | | | | | | | | |
| Annexin | | | | | | | | |
| V−) | | | | | | | | |

TABLE 5B

| | | | | TRAIL | TRAIL | | | |
|---|---|---|---|---|---|---|---|---|
| | Cells + | | TRAIL | Porous | Smooth | TRAIL | | |
| | 0.01% | Blank | encapsulated | Hydro | Hydro | Magnetic | Soluble | 100 nM |
| 48 Hrs. | DMSO | beads | PLGA | gels | gels | Beads | TRAIL | Camptothecin |
| Live Cells | 77.8 | 82.6 | 85.6 | 12.0 | 36.0 | 49.7 | 21.3 | 3.36 |
| (7-AAD−/ | | | | | | | | |
| Annexin | | | | | | | | |
| V−) | | | | | | | | |
| Early Stage | 7.01 | 4.93 | 3.10 | 22.5 | 18.3 | 13.8 | 20.4 | 25.5 |
| Apoptotic | | | | | | | | |
| (7-AAD−/ | | | | | | | | |
| Annexin | | | | | | | | |
| V+) | | | | | | | | |
| Late Stage | 9.54 | 8.10 | 5.70 | 63.5 | 43.0 | 32.9 | 56.9 | 69.8 |
| Apoptotic | | | | | | | | |
| (7-AAD+/ | | | | | | | | |
| Annexin | | | | | | | | |
| V+) | | | | | | | | |
| Necrotic | 5.62 | 4.39 | 5.61 | 2.10 | 2.67 | 3.61 | 1.42 | 1.33 |
| (7-AAD+/ | | | | | | | | |
| Annexin | | | | | | | | |
| V−) | | | | | | | | |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

### NUMBERED EMBODIMENTS

The following list of embodiments is included herein for illustration purposes only and is not intended to be comprehensive or limiting. The subject matter to be claimed is expressly not limited to the following embodiments.

Embodiment 1. A synthetic cytotoxic cell, comprising
a) a hydrogel microsphere with a surface; and
b) a TNF-related apoptosis-inducing ligand (TRAIL), or active fragment thereof, wherein the TRAIL or active fragment thereof is attached to the surface of the hydrogel microsphere.
   Embodiment 2. The synthetic cytotoxic cell of embodiment 1, wherein the hydrogel microsphere is a porous hydrogel microsphere.
   Embodiment 3. The synthetic cytotoxic cell of embodiment 1, wherein the hydrogel microsphere is a smooth hydrogel microsphere.
   Embodiment 4. The synthetic cytotoxic cell of any one of embodiments 1-3, wherein the hydrogel microsphere comprises polymerized acrylamide and bis-acrylamide.
   Embodiment 5. The synthetic cytotoxic cell of any one of embodiments 1-4, wherein the hydrogel microsphere is comprised of poly (lactic-co-glycolic acid).
   Embodiment 6. The synthetic cytotoxic cell of embodiment 5, wherein the ratio of lactide to glycolide is between 90:10 to 50:50.
   Embodiment 7. The synthetic cytotoxic cell of embodiment 6, wherein the ratio of lactide to glycolide is about 85:15.
   Embodiment 8. The synthetic cytotoxic cell of any one of embodiments 1-7, wherein the hydrogel microsphere is comprised of a biodegradable polymer.
   Embodiment 9. The synthetic cytotoxic cell of any one of embodiments 1-8, wherein the TRAIL or active fragment thereof comprises an amino acid sequence having at least 80%, at least 85%, at least 90%, at least 95%, at least 96%, at least 97%, at least 98%, at least 99%, or 100% identity to SEQ ID NO: 1.
   Embodiment 10. The synthetic cytotoxic cell of any one of embodiments 1-8, wherein the TRAIL or active fragment thereof comprises an amino acid sequence having at least 80%, at least 85%, at least 90%, at least 95%, at least 96%, at least 97%, at least 98%, at least 99%, or 100% identity to SEQ ID NO: 2.
   Embodiment 11. The synthetic cytotoxic cell of any one of embodiments 1-10, wherein the TRAIL or active fragment thereof is linked to an Fc domain.
   Embodiment 12. The synthetic cytotoxic cell of any one of embodiments 1-11, wherein the TRAIL or active fragment thereof is biotinylated.
   Embodiment 13. The synthetic cytotoxic cell of any one of embodiments 1-12, wherein the surface of the microsphere comprises a streptavidin molecule.
   Embodiment 14. The synthetic cytotoxic cell of any one of embodiments 1-13, wherein the TRAIL or active fragment thereof is biotinylated and wherein the surface of the hydrogel microsphere comprises a streptavidin molecule.
   Embodiment 15. The synthetic cytotoxic cell of embodiment 14, wherein the biotinylated TRAIL or active fragment thereof is attached to the surface of the hydrogel microsphere via interaction with the streptavidin molecule.
   Embodiment 16. The synthetic cytotoxic cell of any one of embodiments 0-15, comprising a multimer of the TRAIL or active fragment thereof.
   Embodiment 17. The synthetic cytotoxic cell of any one of embodiments 0-16, comprising a trimer of the TRAIL or active fragment thereof.
   Embodiment 18. The synthetic cytotoxic cell of embodiment 16, wherein the multimer comprises a first TRAIL or active fragment thereof and a second TRAIL or active fragment thereof, wherein a linker attaches the first TRAIL or active fragment thereof, to the second TRAIL or active fragment thereof.
   Embodiment 19. The synthetic cytotoxic cell of embodiment 18, wherein the linker comprises an amino acid residue.
   Embodiment 20. The synthetic cytotoxic cell of embodiment 19, wherein the linker comprises a seine residue and/or a glycine residue.
   Embodiment 21. The synthetic cytotoxic cell of any one of embodiments 0-20, wherein the diameter of the synthetic cytotoxic cell ranges from about 1 μm to about 45 μm.
   Embodiment 22. The synthetic cytotoxic cell of embodiment 21, wherein the diameter of the synthetic cytotoxic cell is about 10 μm.
   Embodiment 23. The synthetic cytotoxic cell of any one of embodiments 0-22, wherein the synthetic cytotoxic cell exhibits a mean Young's modulus in the range of about 0.2 kPa and about 400 kPa.
   Embodiment 24. A population of the synthetic cytotoxic cell of any one of embodiments 1-23, wherein the polydispersity index of the population is less than 0.05.
   Embodiment 25. A composition, comprising the synthetic cytotoxic cell of any one of embodiments 1-23.
   Embodiment 26. A pharmaceutical composition, comprising the synthetic cytotoxic cell of any one of embodiments 1-23 and a pharmaceutically acceptable excipient, carrier, or diluent.
   Embodiment 27. A method of inducing apoptosis and/or necrosis of a target cell, comprising contacting the target cell with the synthetic cytotoxic cell of any one of embodiments 0-23, the population of embodiment 24, the composition of embodiment 25, or the pharmaceutical composition of embodiment 26, thereby inducing apoptosis and/or necrosis of the target cell.
   Embodiment 28. The method of embodiment 27, wherein the contacting of the target cell is performed in vitro, ex vivo, or in vivo.
   Embodiment 29. The method of embodiment 28, wherein the contacting of the target cell is performed in vivo.
   Embodiment 30. The method of embodiment 29, comprising administering the synthetic cytotoxic cell or the composition to a subject in need thereof.
   Embodiment 31. A method of inducing apoptosis and/or necrosis of a target cell in a subject in need thereof, comprising administering the synthetic cytotoxic cell of any one of embodiments 0-23, the population of embodiment 24, the composition of embodiment 25, or the pharmaceutical composition of embodiment 26 to the subject, thereby inducing apoptosis and/or necrosis of the target cell in the subject.
   Embodiment 32. The method of embodiment 31, wherein the administering results in contacting of the target cell with the synthetic cytotoxic cell in vivo.
   Embodiment 33. A method of treating a cancer in a subject in need thereof, comprising administering the synthetic cytotoxic cell of any one of embodiments 0-23, the population of embodiment 24, the composition of embodiment 25, or the pharmaceutical composition of embodiment 26 to the subject.
   Embodiment 34. The method of embodiment 33, wherein the method comprises inducing apoptosis and/or necrosis of a target cell in the subject.
   Embodiment 35. The method of any one of embodiments 27-34, wherein the target cell expresses one or both of: (a) a TRAIL receptor DR4; and (b) a TRAIL receptor DR5.
   Embodiment 36. The method of embodiment 35, wherein the cell expresses a TRAIL receptor DR4.
   Embodiment 37. The method of embodiment 36, wherein the TRAIL receptor DR4 comprises an amino acid sequence having at least 80%, at least 85%, at least 90%, at least 95%, at least 96%, at least 97%, at least 98%, at least 99%, or 100% identity to SEQ ID NO: 3.
   Embodiment 38. The method of embodiment 35, wherein the cell expresses a TRAIL receptor DR5.
   Embodiment 39. The method of embodiment 38, wherein the TRAIL receptor DR5 comprises an amino acid sequence having at least 80%, at least 85%, at least 90%, at least 95%, at least 96%, at least 97%, at least 98%, at least 99%, or 100% identity to SEQ ID NO: 4.
   Embodiment 40. The method of any one of embodiments 35-39, wherein the method results in an interaction between: (i) TRAIL or active fragment thereof and DR4; (ii) TRAIL or active fragment thereof and DR5; or (iii) both (i) and (ii).
   Embodiment 41. The method of any one of embodiments 33-40, wherein the cancer is a leukemia, a lymphoma, a myeloma, a carcinoma, a sarcoma, a brain cancer, a spinal cord cancer, chronic lymphocytic leukemia (CLL), B cell acute lymphocytic leukemia (B-ALL), acute lymphoblastic leukemia (ALL), acute myeloid leukemia (AML), non-Hodgkin's lymphoma (NHL), diffuse large cell lymphoma (DLCL), diffuse large B cell lymphoma (DLBCL), Hodgkin's lymphoma, multiple myeloma, renal cell carcinoma (RCC), hepatocellular carcinoma, melanoma, mesothelioma, colorectal cancer, bladder cancer, breast cancer, colorectal cancer, ovarian cancer, prostate cancer, lung cancer, esophageal cancer, pancreatic cancer, head and neck cancer, liver cancer, cervical cancer, breast cancer, astrocytoma, medulloblastoma, neuroblastoma, non-small cell lung cancer, peritoneal carcinomatosis, a solid tumor, malignant pleura mesothelioma, gastric cancer, urothelial cancer, cholangiocarcinoma, hepatocellular carcinoma, or any combination thereof.
   Embodiment 42. The method of any one of embodiment 30-41, wherein the subject is human.
   Embodiment 43. The method of any one of embodiments 27-42, wherein the method results in a proportion of apoptotic and/or necrotic target cells that is higher than the proportion of apoptotic and/or necrotic target cells resulting from contacting the target cell with a soluble form of TRAIL or active fragment thereof.
   Embodiment 44. The method of any one of embodiments 27-43, wherein the method results in a proportion of apoptotic and/or necrotic target cells that is higher than the proportion of apoptotic and/or necrotic target cells resulting from contacting the target cell with a comparator microsphere, wherein the comparator microsphere comprises a non-hydrogel microsphere and the TRAIL or active fragment thereof, wherein the TRAIL or active fragment thereof is attached to the surface of the non-hydrogel microsphere.
   Embodiment 45. The method of any one of embodiments 27-44, wherein the method results in a proportion of apoptotic and/or necrotic target cells that is higher than the proportion of apoptotic and/or necrotic target cells resulting from contacting the target cell with a comparator microsphere, wherein the comparator microsphere comprises a microsphere and TRAIL or active fragment thereof, wherein the TRAIL or active fragment thereof is encapsulated by the microsphere.
   Embodiment 46. The method of embodiment 45, wherein comparator microsphere comprises a microsphere comprised of poly (lactic-co-glycolic acid).
   Embodiment 47. The method of any one of embodiments 27-46, wherein the target cell is a cancer cell.
   Embodiment 48. A conjugate comprising a cell and the synthetic cytotoxic cell of any one of embodiments 1-23.
   Embodiment 49. A cell, wherein the cell is conjugated to the synthetic cytotoxic cell of any one of embodiments 1-23.
   Embodiment 50. A mixture of (i) a cell, and a (ii) the synthetic cytotoxic cell of any one of embodiments 1-23.
   Embodiment 51. The conjugate of embodiment 48, the cell of embodiment 49, or the mixture of embodiment 50, wherein the cell and the synthetic cytotoxic cell are non-covalently conjugated.
   Embodiment 52. The conjugate of embodiment 48 or 51, the cell of embodiment 49 or 51, or the mixture of embodiment 50 or 51, wherein the cell expresses one or both of: (a) a TRAIL receptor DR4 and (b) a TRAIL receptor DR5.
   Embodiment 53. The conjugate, the cell, or the mixture of embodiment 52, wherein the cell expresses a TRAIL receptor DR4.
   Embodiment 54. The conjugate, the cell, or the mixture of embodiment 53, wherein the TRAIL receptor DR4 comprises an amino acid sequence having at least 80%, at least 85%, at least 90%, at least 95%, at least 96%, at least 97%, at least 98%, at least 99%, or 100% identity to SEQ ID NO: 3.
   Embodiment 55. The conjugate, the cell, or the mixture of embodiment 52, wherein the cell expresses a TRAIL receptor DR5, or an antigen binding fragment thereof.
   Embodiment 56. The conjugate, the cell, or the mixture of embodiment 55, wherein the TRAIL receptor DR5 comprises an amino acid sequence having at least 80%, at least 85%, at least 90%, at least 95%, at least 96%, at least 97%, at least 98%, at least 99%, or 100% identity to SEQ ID NO: 4.
   Embodiment 57. The conjugate of any one of embodiments 52-56, the cell of any one of embodiments 52-56, or the mixture of any one of embodiments 52-56, wherein the cell is conjugated to the synthetic cytotoxic cell via an interaction between: (i) the TRAIL or active fragment thereof and DR4; (ii) the TRAIL or active fragment thereof and DR5; or (iii) both (i) and (ii).
   Embodiment 58. The conjugate, the cell, or the mixture of embodiment 57, wherein the interaction between the TRAIL or active fragment thereof, and one or both of DR4 and DR5 results in the induction of apoptosis and/or necrosis of the cell.
   Embodiment 59. The conjugate, the cell, or the mixture of embodiment 58, wherein the cell is a cancer cell.
   Embodiment 60. A synthetic cytotoxic cell, comprising a porous hydrogel microsphere and a TNF-related apoptosis-inducing ligand (TRAIL), or active fragment thereof, wherein the TRAIL or active fragment thereof is attached to the surface of the hydrogel microsphere.
   Embodiment 61. The synthetic cytotoxic cell of embodiment 60, wherein the hydrogel microsphere comprises polymerized acrylamide and bis-acrylamide.
   Embodiment 62. The synthetic cytotoxic cell of embodiment 60 or embodiment 61, wherein the TRAIL or active fragment thereof comprises an amino acid sequence having at least 80%, at least 85%, at least 90%, at least 95%, at least 96%, at least 97%, at least 98%, at least 99%, or 100% identity to SEQ ID NO: 1.
   Embodiment 63. The synthetic cytotoxic cell of any one of embodiments 60-62, wherein the TRAIL or active fragment thereof is linked to an Fc domain.
   Embodiment 64. The synthetic cytotoxic cell of any one of embodiments 60-63, wherein the TRAIL or active fragment thereof is biotinylated.
   Embodiment 65. The synthetic cytotoxic cell of any one of embodiments 60-64, wherein the surface of the microsphere comprises a streptavidin molecule.
   Embodiment 66. The synthetic cytotoxic cell of any one of embodiments 60-65, wherein the TRAIL or active fragment thereof is biotinylated and wherein the surface of the hydrogel microsphere comprises a streptavidin molecule.
   Embodiment 67. The synthetic cytotoxic cell of embodiment 66, wherein the biotinylated TRAIL or active fragment thereof is attached to the surface of the hydrogel microsphere via interaction with the streptavidin molecule.
   Embodiment 68. The synthetic cytotoxic cell of any one of embodiments 60-67, comprising a trimer of the TRAIL or active fragment thereof.
   Embodiment 69. A composition, comprising the synthetic cytotoxic cell of any one of embodiments 60-68.
   Embodiment 70. A pharmaceutical composition, comprising the synthetic cytotoxic cell of any one of embodiments 60-68 and a pharmaceutically acceptable excipient, carrier, or diluent.
   Embodiment 71. A method of inducing apoptosis and/or necrosis of a target cell, comprising contacting the target cell with the synthetic cytotoxic cell of any one of embodiments 60-68, the composition of embodiment 69, or the pharmaceutical composition of embodiment 70, thereby inducing apoptosis and/or necrosis of the target cell.
   Embodiment 72. The method of embodiment 71, wherein the contacting of the target cell is performed in vitro, ex vivo, or in vivo.
   Embodiment 73. The method of embodiment 72, wherein the contacting of the target cell is performed in vivo.
   Embodiment 74. The method of embodiment 73, comprising administering the synthetic cytotoxic cell, the composition, or the pharmaceutical composition to a subject in need thereof.
   Embodiment 75. A method of inducing apoptosis and/or necrosis of a target cell in a subject in need thereof, comprising administering the synthetic cytotoxic cell of any one of embodiments 60-68, the composition of embodiment 69, or the pharmaceutical composition of embodiment 70 to the subject, thereby inducing apoptosis and/or necrosis of the target cell in the subject.
   Embodiment 76. The method of embodiment 75, wherein the administering results in contacting of the target cell with the synthetic cytotoxic cell in vivo.
   Embodiment 77. A method of treating a cancer in a subject in need thereof, comprising administering the synthetic cytotoxic cell of any one of embodiments 60-68, the composition of embodiment 69, or the pharmaceutical composition of embodiment 70 to the subject.
   Embodiment 78. The method of embodiment 77, wherein the method comprises inducing apoptosis and/or necrosis of a target cell in the subject.
   Embodiment 79. The method of any one of embodiments 71-78, wherein the target cell expresses one or both of: (a) a TRAIL receptor DR4, or an antigen binding fragment thereof; and (b) a TRAIL receptor DR5, or an antigen binding fragment thereof.
   Embodiment 80. The method of embodiment 79, wherein the cell expresses a TRAIL receptor DR4, or an antigen binding fragment thereof.
   Embodiment 81. The method of embodiment 80, wherein the TRAIL receptor DR4, or the antigen binding fragment thereof comprises an amino acid sequence having at least 80%, at least 85%, at least 90%, at least 95%, at least 96%, at least 97%, at least 98%, at least 99%, or 100% identity to SEQ ID NO: 3.
   Embodiment 82. The method of embodiment 79, wherein the cell expresses a TRAIL receptor DR5, or an antigen binding fragment thereof.
   Embodiment 83. The method of embodiment 82, wherein the TRAIL receptor DR5, or the antigen binding fragment thereof comprises an amino acid sequence having at least 80%, at least 85%, at least 90%, at least 95%, at least 96%, at least 97%, at least 98%, at least 99%, or 100% identity to SEQ ID NO: 4.
   Embodiment 84. The method of any one of embodiments 79-83, wherein the method results in an interaction between: (i) the TNF-related apoptosis-inducing ligand (TRAIL), or active fragment thereof and a TRAIL receptor DR4; (ii) the TNF-related apoptosis-inducing ligand (TRAIL), or active fragment thereof and a TRAIL receptor DR5; (iii) both (i) and (ii).
   Embodiment 85. The method of any one of embodiments 77-84, wherein the cancer is a leukemia, a lymphoma, a myeloma, a carcinoma, a sarcoma, a brain cancer, a spinal cord cancer, chronic lymphocytic leukemia (CLL), B cell acute lymphocytic leukemia (B-ALL), acute lymphoblastic leukemia (ALL), acute myeloid leukemia (AML), non-Hodgkin's lymphoma (NHL), diffuse large cell lymphoma (DLCL), diffuse large B cell lymphoma (DLBCL), Hodgkin's lymphoma, multiple myeloma, renal cell carcinoma (RCC), hepatocellular carcinoma, melanoma, mesothelioma, colorectal cancer, bladder cancer, breast cancer, colorectal cancer, ovarian cancer, prostate cancer, lung cancer, esophageal cancer, pancreatic cancer, head and neck cancer, liver cancer, cervical cancer, breast cancer, astrocytoma, medulloblastoma, neuroblastoma, non-small cell lung cancer, peritoneal carcinomatosis, a solid tumor, malignant pleura mesothelioma, gastric cancer, urothelial cancer, cholangiocarcinoma, hepatocellular carcinoma, or any combination thereof.
   Embodiment 86. The method of any one of embodiments 74-85, wherein the subject is human.
   Embodiment 87. The method of any one of embodiments 71-86, wherein the method results in a proportion of apoptotic and/or necrotic target cells that is higher than the proportion of apoptotic and/or necrotic target cells resulting from contacting the target cell with a soluble form of TRAIL or active fragment thereof.
   Embodiment 88. The method of any one of embodiments 71-87, wherein the method results in a proportion of apoptotic and/or necrotic target cells that is higher than the proportion of apoptotic and/or necrotic target cells resulting from contacting the target cell with a comparator microsphere, wherein the comparator microsphere comprises a non-hydrogel microsphere and a TNF-related apoptosis-inducing ligand (TRAIL), or active fragment thereof, wherein the TRAIL or active fragment thereof is attached to the surface of the non-hydrogel microsphere.
   Embodiment 89. The method of any one of embodiments 71-88, wherein the method results in a proportion of apoptotic and/or necrotic target cells that is higher than the proportion of apoptotic and/or necrotic target cells resulting from contacting the target cell with a comparator microsphere, wherein the comparator microsphere comprises a microsphere and a TNF-related apoptosis-inducing ligand (TRAIL), or active fragment thereof, wherein the TRAIL or active fragment thereof is encapsulated by the microsphere, optionally, wherein the microsphere is comprised of poly (lactic-co-glycolic acid).
   Embodiment 90. The method of any one of embodiments 71-89, wherein the method results in a proportion of apoptotic and/or necrotic target cells that is higher than the proportion of apoptotic and/or necrotic target cells resulting from contacting the target cell with a comparator microsphere, wherein the comparator microsphere comprises a smooth porous microsphere and a TNF-related apoptosis-inducing ligand (TRAIL), or active fragment thereof, wherein the TRAIL or active fragment thereof is attached to the surface of the smooth porous microsphere.
   Embodiment 91. The method of any one of embodiments 71-90, wherein the method results in a proportion of early apoptotic target cells that is higher than the proportion of early or late apoptotic target cells resulting from contacting the target cell with a soluble form of TRAIL or active fragment thereof.
   Embodiment 92. The method of any one of embodiments 71-91, wherein the method results in a proportion of early apoptotic target cells that is higher than the proportion of early or late apoptotic target cells resulting from contacting the target cell with a comparator microsphere, wherein the comparator microsphere comprises a non-hydrogel microsphere and a TNF-related apoptosis-inducing ligand (TRAIL), or active fragment thereof, wherein the TRAIL or active fragment thereof is attached to the surface of the non-hydrogel microsphere.
   Embodiment 93. The method of any one of embodiments 71-92, wherein the method results in a proportion of early apoptotic target cells that is higher than the proportion of early or late apoptotic target cells resulting from contacting the target cell with a comparator microsphere, wherein the comparator microsphere comprises a microsphere and a TNF-related apoptosis-inducing ligand (TRAIL), or active fragment thereof, wherein the TRAIL or active fragment thereof is encapsulated by the microsphere, optionally, wherein the microsphere is comprised of poly (lactic-co-glycolic acid).
   Embodiment 94. The method of any one of embodiments 71-93, wherein the method results in a proportion of early apoptotic target cells that is higher than the proportion of early or late apoptotic target cells resulting from contacting the target cell with a comparator microsphere, wherein the comparator microsphere comprises a smooth porous microsphere and a TNF-related apoptosis-inducing ligand (TRAIL), or active fragment thereof, wherein the TRAIL or active fragment thereof is attached to the surface of the smooth porous microsphere.
   Embodiment 95. The method of any one of embodiments 71-94, wherein the method results in a proportion of late apoptotic target cells that is higher than the proportion of early or late apoptotic target cells resulting from contacting the target cell with a soluble form of TRAIL or active fragment thereof.
   Embodiment 96. The method of any one of embodiments 71-95, wherein the method results in a proportion of late apoptotic target cells that is higher than the proportion of early or late apoptotic target cells resulting from contacting the target cell with a comparator microsphere, wherein the comparator microsphere comprises a non-hydrogel microsphere and a TNF-related apoptosis-inducing ligand (TRAIL), or active fragment thereof, wherein the TRAIL or active fragment thereof is attached to the surface of the non-hydrogel microsphere.
   Embodiment 97. The method of any one of embodiments 71-96, wherein the method results in a proportion of late apoptotic target cells that is higher than the proportion of early or late apoptotic target cells resulting from contacting the target cell with a comparator microsphere, wherein the comparator microsphere comprises a microsphere and a TNF-related apoptosis-inducing ligand (TRAIL), or active fragment thereof, wherein the TRAIL or active fragment thereof is encapsulated by the microsphere, optionally, wherein the microsphere is comprised of poly (lactic-co-glycolic acid).
   Embodiment 98. The method of any one of embodiments 71-97, wherein the method results in a proportion of late apoptotic target cells that is higher than the proportion of early or late apoptotic target cells resulting from contacting the target cell with a comparator microsphere, wherein the comparator microsphere comprises a smooth porous microsphere and a TNF-related apoptosis-inducing ligand (TRAIL), or active fragment thereof, wherein the TRAIL or active fragment thereof is attached to the surface of the smooth porous microsphere.
   Embodiment 99. A synthetic cytotoxic cell, comprising:
a) a hydrogel microsphere with a surface; and
b) a TRAIL receptor selected from the group consisting of DR4 and DR5;
wherein the TRAIL receptor is tethered to the surface of the hydrogel microsphere.
   Embodiment 100. The synthetic cytotoxic cell of embodiment 99, wherein the TRAIL receptor is further tethered to a biological cell.
   Embodiment 101. A biological complex, comprising:
a) a hydrogel microsphere with a surface; and
b) a biological cell comprising a TRAIL receptor selected from the group consisting of DR4 and DR5;
wherein the biological cell is tethered to the hydrogel microsphere via the TRAIL receptor.
   Embodiment 102. The synthetic cytotoxic cell of embodiments 99 or 100 or the biological complex of embodiment 101, wherein the TRAIL receptor is tethered to the surface of the hydrogel microsphere via a linker.
   Embodiment 103. The synthetic cytotoxic cell or biological complex of embodiment 102, wherein the linker comprises a biotin/streptavidin complex.
   Embodiment 104. The synthetic cytotoxic cell or biological complex of embodiments 102 or 103, wherein the TRAIL receptor is covalently linked to the linker.
   Embodiment 105. The synthetic cytotoxic cell or biological complex of embodiments 102 or 103, wherein the TRAIL receptor is non-covalently linked to the linker.
   Embodiment 106. The synthetic cytotoxic cell or biological complex of any one of embodiments 99-105, wherein the TRAIL receptor DR4 comprises an amino acid sequence having at least 80%, at least 85%, at least 90%, at least 95%, at least 96%, at least 97%, at least 98%, at least 99%, or 100% identity to SEQ ID NO: 3.
   Embodiment 107. The synthetic cytotoxic cell or biological complex of any one of embodiments 99-106, wherein the TRAIL receptor DR5 comprises an amino acid sequence having at least 80%, at least 85%, at least 90%, at least 95%, at least 96%, at least 97%, at least 98%, at least 99%, or 100% identity to SEQ ID NO: 4.
   Embodiment 108. The synthetic cytotoxic cell or biological complex of any one of embodiments 99-107, wherein a TRAIL or active fragment thereof is attached to the surface of the hydrogel microsphere.
   Embodiment 109. The synthetic cytotoxic cell or biological complex of any one of embodiments 99-108, wherein the surface of the hydrogel microsphere comprises a streptavidin molecule.
   Embodiment 110. The synthetic cytotoxic cell or biological complex of embodiment 109, wherein the TRAIL or active fragment thereof is biotinylated.
   Embodiment 111. The synthetic cytotoxic cell or biological complex of embodiment 110, wherein the biotinylated TRAIL or active fragment thereof is bound to the streptavidin molecule on the surface of the hydrogel microsphere.
   Embodiment 112. The synthetic cytotoxic cell or biological complex of any one of embodiments 108-111, wherein the TRAIL receptor is tethered to the surface of the hydrogel microsphere via an interaction between the TRAIL or the active fragment thereof, and the TRAIL receptor.

1. A synthetic cytotoxic cell, comprising
a) a hydrogel microsphere with a surface; and
b) a TNF-related apoptosis-inducing ligand (TRAIL), or active fragment thereof, wherein the TRAIL or active fragment thereof is attached to the surface of the hydrogel microsphere.

2. The synthetic cytotoxic cell of claim 1, wherein the hydrogel microsphere is a porous hydrogel microsphere.

3. The synthetic cytotoxic cell of claim 1, wherein the hydrogel microsphere is a smooth hydrogel microsphere.

4. The synthetic cytotoxic cell of claim 1, wherein the TRAIL or active fragment thereof comprises an amino acid sequence having at least 80% sequence identity to SEQ ID NO: 2.

5. The synthetic cytotoxic cell of claim 1, wherein the TRAIL or active fragment thereof is biotinylated, wherein the surface of the hydrogel microsphere comprises a streptavidin molecule, and wherein the biotinylated TRAIL or active fragment thereof is attached to the surface of the hydrogel microsphere via interaction with the streptavidin molecule.

6. The synthetic cytotoxic cell of claim 1, comprising a trimer of the TRAIL or active fragment thereof.

7. A pharmaceutical composition, comprising the synthetic cytotoxic cell of claim 1 and a pharmaceutically acceptable excipient, carrier, or diluent.

8. A method of inducing one or both of apoptosis and necrosis of a target cell, comprising contacting the target cell with the synthetic cytotoxic cell, thereby inducing one or both of apoptosis and necrosis of the target cell, wherein the synthetic cytotoxic cell comprises:
a) a hydrogel microsphere with a surface; and
b) a TNF-related apoptosis-inducing ligand (TRAIL), or active fragment thereof, wherein the TRAIL or active fragment thereof is attached to the surface of the hydrogel microsphere.

9. The method of claim 8, wherein the target cell expresses one or both of: (a) a TRAIL receptor DR4; and (b) a TRAIL receptor DR5,
wherein the TRAIL receptor DR4 comprises an amino acid sequence having at least 80% sequence identity to SEQ ID NO: 3, and
wherein the TRAIL receptor DR5 comprises an amino acid sequence having at least 80% sequence identity to SEQ ID NO: 4.

10. The method of claim 8, wherein the method results in a proportion of one or both of apoptotic and necrotic target cells that is:
a. higher than the proportion of one or both of apoptotic and necrotic target cells resulting from contacting the target cell with a soluble form of TRAIL or active fragment thereof,
b. higher than the proportion of one or both of apoptotic and necrotic target cells resulting from contacting the target cell with a comparator microsphere, wherein the comparator microsphere comprises a non-hydrogel microsphere and a TRAIL or active fragment thereof, wherein the TRAIL or active fragment thereof is attached to the surface of the non-hydrogel microsphere, or
c. higher than the proportion of one or both of apoptotic and necrotic target cells resulting from contacting the target cell with a comparator microsphere, wherein the comparator microsphere comprises a microsphere and a TRAIL or active fragment thereof, wherein the TRAIL or active fragment thereof is encapsulated by the microsphere, wherein the comparator microsphere comprises poly (lactic-co-glycolic acid).

11. A biological complex, comprising:
a) a hydrogel microsphere with a surface; and
b) a biological cell comprising a TRAIL receptor selected from the group consisting of DR4 and DR5;
wherein the biological cell is tethered to the hydrogel microsphere via the TRAIL receptor.

12. The biological complex of claim 11, wherein a TRAIL or active fragment thereof is attached to the surface of the hydrogel microsphere.

13. The synthetic cytotoxic cell of claim 6, wherein the trimer comprises a first TRAIL or active fragment thereof and a second TRAIL or active fragment thereof, wherein a linker attaches the first TRAIL or active fragment thereof to the second TRAIL or active fragment thereof.

14. The synthetic cytotoxic cell of claim 1, wherein the hydrogel microsphere comprises polymerized acrylamide and bis-acrylamide.

15. The method of claim 8, wherein the hydrogel microsphere is a porous hydrogel microsphere.

16. The method of claim 8, wherein the TRAIL or active fragment thereof comprises an amino acid sequence having at least 80% sequence identity to SEQ ID NO: 2.

17. The method of claim 8, wherein the TRAIL or active fragment thereof is biotinylated, wherein the surface of the hydrogel microsphere comprises a streptavidin molecule, and wherein the biotinylated TRAIL or active fragment thereof is attached to the surface of the hydrogel microsphere via interaction with the streptavidin molecule.

18. The method of claim 8, comprising a trimer of the TRAIL or active fragment thereof.

19. The method of claim 18, wherein the trimer comprises a first TRAIL or active fragment thereof and a second TRAIL or active fragment thereof, wherein a linker attaches the first TRAIL or active fragment thereof to the second TRAIL or active fragment thereof.

20. The method of claim 8, wherein the hydrogel microsphere comprises polymerized acrylamide and bis-acrylamide.
